SEQUENCE LISTING (20 sequences)

SEQ ID NO: 1 - protein, 7 aa, Simian virus 40
PKKKRKV

SEQ ID NO: 2 - protein, 10 aa, Simian virus 40
PKKKRKVEAS

SEQ ID NO: 3 - protein, 16 aa, Artificial Sequence
  Synthetic nucleoplasmin bipartite NLS
KRPAATKKAGQAKKKK

SEQ ID NO: 4 - protein, 9 aa, Homo sapiens
PAAKRVKLD

SEQ ID NO: 5 - protein, 11 aa, Homo sapiens
RQRRNELKRSP

SEQ ID NO: 6 - protein, 38 aa, Homo sapiens
NQSSNFGPMKGGNFGGRSSGPYGGGGQYFAKPRNQGGY

SEQ ID NO: 7 - protein, 42 aa, Homo sapiens
RMRIZFKNKGKDTAELRRRRVEVSVELRKAKKDEQILKRRNV

SEQ ID NO: 8 - protein, 8 aa, Homo sapiens
VSRKRPRP

SEQ ID NO: 9 - protein, 8 aa, Homo sapiens
PPKKARED

SEQ ID NO: 10 - protein, 8 aa, Homo sapiens
PQPKKKPL

SEQ ID NO: 11 - protein, 12 aa, Mus sp.
SALIKKKKKMAP

SEQ ID NO: 12 - protein, 5 aa, Influenza virus
DRLRR

SEQ ID NO: 13 - protein, 7 aa, Influenza virus
PKQKKRK

SEQ ID NO: 14 - protein, 10 aa, Hepatitis D virus
RKLKKKIKKL

SEQ ID NO: 15 - protein, 10 aa, Mus sp.
REKKKFLKRR

SEQ ID NO: 16 - protein, 20 aa, Homo sapiens
KRKGDEVDGVDEVAKKKSKK

SEQ ID NO: 17 - protein, 17 aa, Homo sapiens
RKCLQAGMNLEARKTKK

SEQ ID NO: 18 - DNA, 54 bp, Artificial Sequence
  Synthetic
  misc_feature (47)..(54): n is a, c, g, or t
gactcgtaatacgactcactatagggacacgacgctcttccgatctnnnnnnnn

SEQ ID NO: 19 - DNA, 34 bp, Artificial Sequence
  Synthetic
  misc_feature (1)..(1): Adenylation-5′
  misc_feature (34)..(34): ddc-3′
agatcggaagagcacacgtctgaactccagtcac

SEQ ID NO: 20 - DNA, 31 bp, Artificial Sequence
  Synthetic
gtgactggagttcagacgtgtgctcttccga

(19) United States
(12) Patent Application Publication
(10) Pub. No.: US 2022/0042097 A1
(43) Pub. Date: Feb. 10, 2022

(54) IN-SITU SPATIAL TRANSCRIPTOMICS AND PROTEOMICS

(71) Applicants: The Broad Institute, Inc., Cambridge, MA (US); Massachusetts Institute of Technology, Cambridge, MA (US)

(72) Inventors: Aviv Regev, Cambridge, MA (US); Sanja Vickovic, Cambridge, MA (US)

(21) Appl. No.: 17/393,994

(22) Filed: Aug. 4, 2021

Related U.S. Application Data
(60) Provisional application No. 63/061,125, filed on Aug. 4, 2020.

(51) Int. Cl. C12Q 1/6881; C12Q 1/6841; C12Q 1/6837; C12N 15/10; C12N 15/11

(52) U.S. Cl. CPC C12Q 1/6881; C12Q 1/6841; C12Q 1/6837; C12N 2310/20; C12N 15/1068; C12N 15/11; C12N 15/1065

(57) ABSTRACT

The present disclosure relates to systems and method of in-situ tissue profiling. Methods for spatiotemporal processing of a sample, capturing molecules of interest, and correlating cells in the sample to the capture molecules are provided.

### CROSS-REFERENCE TO RELATED APPLICATIONS

This application claims the benefit of and priority to U.S. Provisional Patent Application No. 63/061,125, filed on Aug. 4, 2020, entitled IN-SITU SPATIAL TRANSCRIPTOMICS AND PROTEOMICS″, the contents of which is incorporated by reference herein in its entirety.

### REFERENCE TO AN ELECTRONIC SEQUENCE LISTING

The contents of the electronic sequence listing (“BROD-5135US ST25.txt”; Size is 4,380 bytes and it was created on Aug. 3, 2021) is herein incorporated by reference in its entirety.

### TECHNICAL FIELD

The subject matter disclosed herein is generally directed to systems and method of in-situ spatial transcriptomics and tissue profiling.

### BACKGROUND

The function of different cell types in the brain results from a combination of their unique molecular profiles and how these govern their reactions to stimuli from both the immediate and distant neighborhoods as well as their respective developmental trajectories. Single-cell transcriptomics assesses the cellular complexity of tissue regions by capturing their molecular profiles. However, single cells are assembled in a complex structural architecture and there is thus need for correlating single-cell expression to morphological entities. Here, Applicants present an improvement to spatial high-throughput RNA-sequencing termed high-density spatial transcriptomics. Spatially barcoded reverse transcription oligonucleotides are coupled to beads that are then ordered in a random but decodable fashion into individual wells. Histological tissue sections can then be RNA-sequenced at 2 μm resolution with over a million barcodes per experiment. High-density spatial transcriptomics thus provides 2D transcriptome profiling for spatial cell typing and differential expression profiling identifying tissue dynamics.

Cells are organized in many hierarchical layers, starting from their local environments in tissues. To enhance our understanding of such complex structures, Applicants need to focus on making massive, parallel and molecular measurements. Key among these is the measurement of the transcriptome, which mediates between the gene-cell regulatory circuitry and the phenotypic characteristics governed by lineage and architecture in a high-throughput fashion.

Today, one can make use of various approaches that make transcriptome measurements at an ever increasing single-cell resolution. These technologies allow analysis of thousands of dissociated individual cells and assign them into diverse cell types and circuits. The connections between transcripts, circuits, and cells are made based on inferences of genotypes and phenotypes and projected onto two-dimensional space. Although these techniques operate at very high throughput, they potentially risk introducing cell manipulation biases that lead to an altered molecular state.

The transcriptome alone, however, does not provide a full picture of cellular identity. The identity of each cell is also governed by its spatiotemporal position and internal population dynamics as a consequence of the signals it receives from its environment. However, cell classification cannot solely be determined by morphology) and a variety of tools are needed in order to validate cell states and their respective properties, many of them focusing on increased resolution or throughput).

Spatial transcriptomics (ST) technology combines spatial and transcriptomic techniques ST is based on depositing spatially barcoded poly(d)T oligonucleotides for capturing mRNA into 100 μm features on a glass slide. However, at 100 μm analysis was based on more generalized large morphological features, with twenty percent of the tissue dynamics captured in the 100 μm features. There remains a need for more detailed understanding of complex tissues, as understanding of the underlying molecular consequences of patterns over large spatial areas in complex tissues, such as the central nervous system (CNS), remains limited. There also exists a need for techniques to facilitate a more detailed understanding of complex tissues.

Citation or identification of any document in this application is not an admission that such document is available as prior art to the present invention.

### SUMMARY

In certain example embodiments, methods of spatiotemporal processing of a sample of a plurality of cells is provided comprising the steps of depositing a plurality of spatial barcodes on a solid substrate, the spatial barcodes each defining an x,y position on the solid substrate and further comprising a capture molecule; depositing the sample of the plurality of cells on the surface of the solid substrate; and capturing material from one or more cells of the plurality of cells with the capture molecule of the spatial barcode, thereby linking the capture material from the one or more cells with the spatial barcode.

In embodiments, the spatial barcode is provided in a droplet. The droplet can comprise a plurality of spatial barcodes, and optionally further comprise CRISPR-Cas systems The spatial barcode can comprise a bead in some embodiments, which can include color-coded beads and conductivity-coded beads. In one aspect, the conductivity-coded bead is deposited on the solid substrate, the solid substrate comprising pre-etched wells.

In some embodiments, the bead can comprise a plurality of spatial barcodes, which in some embodiments comprise oligonucleotides.

Methods of depositing the spatial barcodes can comprise inkjet, contact printing or fluorescence activated cell sorting (FACS) technologies. In certain embodiments, the depositing is random or ordered.

The step of depositing the spatial barcode comprises the binding of the spatial barcode to the solid substrate in certain embodiments. The binding of the spatial barcode to the solid substrate can be covalent or non-covalent bonding. In embodiments, the solid substrate comprises a surface with available active groups that facilitate the bonding of the spatial barcode to the solid substrate surface.

In embodiments wherein the spatial barcode comprises an oligonucleotide sequence, methods can include building the spatial barcode on the solid substrate, or on a bead.

The method of claim 15, wherein the building the spatial barcode comprises bridge polymerase chain reaction (PCR) or ligation and extension PCR. Methods can comprise building the spatial barcode comprising distributing oligonucleotide sequences on the solid substrate, adding padlock probes, and amplifying and decoding the oligonucleotides on the surface.

In embodiments, the capture molecule comprises target molecule specific sequence, a Tn5 sequence, a 16S sequence, a poly(d)T sequence, a random hexamer sequence, a trypsin molecule, an antibody, a Protein Epitope Signature Tag (PrEST) sequence, or a combination thereof. Preferred embodiments comprise a combination of capture molecules, and in certain embodiments, target-specific molecules such as single nucleotide polymorphisms (SNPs), particular genes or mutations of interest.

The oligonucleotide sequence can further comprise one or more of a unique molecular identifier (UMI), an adapter sequence, a guide sequence. and a primer sequence.

The methods disclosed herein can further comprise the step of decoding the spatial barcode, the decoding comprising sequentially hybridization, in situ sequencing, laser scanning, DNA microscopy. Methods can also comprise sequencing the captured material and/or releasing the captured material. In embodiments, the spatial barcode comprises a cleavable linker. In embodiments, the cleavable linker is a restriction site, and releasing the captured material comprises utilizing a restriction enzyme specific to the restriction site and cleaving the captured molecule. The linker in particular embodiments is enzymatically, thermally or chemically cleavable.

In certain example embodiments, the spatial barcode and the captured material are oligonucleotides, and the releasing comprises synthesizing a complementary strand to the spatial barcode and captured oligonucleotide using a polymerase and releasing the complementary strand or the spatial barcode and captured material oligonucleotide.

In some embodiments, the plurality of cells is a tissue sample. In one preferred embodiment, the tissue sample is greater than about 0.5 cm in thickness, is a biopsy sample, and/or from a mammal. In particular embodiments, the tissue sample is from the central nervous system.

The solid substrate can in some embodiments comprise a glass slide, a polymer, an imaging fiber, or other conductive surface. In embodiments, the solid substrate comprises an array of microwells. In one embodiment, the solid substrate comprises a plurality of microwells in an array, the microwells each about 2 μm, optionally with a 3 μm distance from center to center of each well. In other embodiments, the solid substrate comprises a plurality of locations spaced about 100 nm.

Methods may also comprise steps of capturing an image of the sample on the solid substrate, further comprising annotating regions of the image of the sample, optionally based on morphology, further comprising correlating the captured material to a position in the sample on the solid substrate or any combination of these steps. In an embodiment, the correlating comprises assigning pixel coordinates to the image and coordinating to the x,y position of the spatial barcode.

Methods can comprise assigning a cell type to cells in the sample. Steps of ablating a single layer of the plurality of cells and performing the step of capturing material from one or more cells of the plurality of cells in a second layer of the cells are also provided.

In certain embodiments, the capture molecule comprises a poly(d)T sequence, and the steps further comprise staining the sample; recording the morphology of the stained sample; permeabilizing the sample; capturing mRNA of the sample with the capture molecule, thereby linking mRNA of the cells of the sample with the spatial barcode; and preparing a library of cDNA molecules from the captured mRNA and the linked spatial barcode. The method can optionally comprise sequencing the library of cDNA molecules and can comprise correlating the cDNA molecule to a position in the sample on the solid substrate. The method can optionally comprise assigning a cell type to the plurality of cells in the sample, the assigning comprising detecting differential expression of the expressed genes to generate a gene signature and identifying cell type based on the gene signature at positions in the sample.

Embodiments can include staining the plurality of cells, optionally comprising fluorescent or bright field staining.

Methods can further comprise depositing a plurality of CRISPR-Cas systems on the solid substrate, the CRISPR-Cas system comprising CRISPR-Cas protein or one or more nucleic acid sequences encoding the CRISPR-Cas protein and a guide sequence capable of hybridizing with a target sequence. In embodiments, the one or more CRISPR-Cas systems are deposited at each defined x,y position on the solid substrate. The guide sequences may be optionally linked to the spatial barcode.

Embodiments of the methods disclosed herein comprise delivering CRISPR-Cas systems to the sample prior to or subsequent to depositing the sample on the solid substrate.

Described in certain example embodiments herein are methods of spatial and/or temporal processing of a sample comprising a plurality of cells comprising: a. depositing a sample comprising a plurality of cells on a fixed addressable array or a decoded bead array, i. wherein the fixed addressable array comprises: a plurality of array probes, each array probe comprising a capture molecule and a spatial barcode, wherein each spatial barcode defines a unique x,y, position of each array probe in the fixed addressable array; ii. wherein the decoded bead array comprises a plurality of conductive beads comprising a plurality of bead probes, each comprising a target molecule and a spatial barcode, wherein the plurality of conductive beads are transiently fixed in spatial position to a first side of the fixed addressable array by an electromagnetic field applied to a second side of the fixed addressable array and wherein the first side and the second side are opposite sides of the fixed addressable array; and b. operatively coupling material from the sample to the plurality of array probes of the fixed addressable array or the plurality of bead probes of the decoded bead array, thereby linking the operatively coupled material from the sample with an x,y position in the fixed addressable array and/or the decoded bead array.

In certain example embodiments, operatively coupling material from the sample comprises: directly and/or indirectly capturing material from the sample by a capture molecule of an array probe that is in spatial proximity to the captured material, thereby linking the captured material from the sample with an x,y position in the fixed addressable array.

In certain example embodiments, directly capturing material from the sample comprises capturing a sample polynucleotide by hybridizing the sample polynucleotide to the capture molecule of the array probe that is in spatial proximity to the sample polynucleotide or binding a labeled recognition molecule to a target present in the sample.

In certain example embodiments, the sample polynucleotide is or comprises DNA.

In certain example embodiments, the sample polynucleotide is or comprises RNA.

In certain example embodiments, indirectly capturing material from the sample comprises i. specifically binding a barcoded recognition molecule to a target present in the sample, wherein the barcoded recognition molecule comprises a recognition molecule barcode; optionally, specifically binding a non-barcoded recognition molecule comprising a detectable label to the target present in the sample; and iii. capturing the barcoded recognition molecule barcode by the capture molecule of the array probe that is in spatial proximity to the target.

In certain example embodiments, the method further comprises copying the captured sample polynucleotide(s), the captured barcoded recognition molecule barcode(s), or both, thereby forming a copied sample polynucleotide(s), a copied barcoded recognition molecule barcode(s), or both.

In certain example embodiments, the method further comprises detecting the copied sample polynucleotides, copied barcoded recognition molecule barcodes, or both.

In certain example embodiments, detecting comprises imaging the copied sample polynucleotides, the copied barcoded recognition molecule barcodes, or both.

In certain example embodiments, the method further comprises capturing an image of the sample on the fixed addressable array.

In certain example embodiments, the method further comprises annotating regions of the image of the sample, optionally based on morphology.

In certain example embodiments, the method further comprises correlating the directly captured material, indirectly captured material, or both to a position in the sample on the fixed addressable array.

In certain example embodiments, comprises assigning pixel coordinates to the image of the sample, image of the copied sample polynucleotides, image of the copied barcoded recognition molecules, or a combination thereof and coordinating the assigned pixel coordinates to the x,y position in the fixed addressable array.

In certain example embodiments, the method further comprises assigning a cell type, cell state, or both to cells in the sample.

In certain example embodiments, the method further comprises staining the sample and optionally recording the morphology of the stained sample.

In certain example embodiments, the method further comprises permeabilizing the sample.

In certain example embodiments, copying the captured sample polynucleotide(s), the captured barcoded recognition molecule barcode(s), or both, comprises incorporating labeled dNTP's into the copied sample polynucleotide(s), a copied barcoded recognition molecule barcode(s), or both.

In certain example embodiments, detecting the copied sample polynucleotides, copied barcoded recognition molecule barcodes, or both comprises detecting the labeled dNTPs incorporated into the copied sample polynucleotide(s), a copied barcoded recognition molecule barcode(s), or both.

In certain example embodiments, detecting the labeled dNTPs incorporated into the copied sample polynucleotide(s), a copied barcoded recognition molecule barcode(s), or both comprises imaging the labeled dNTPs.

In certain example embodiments, copying the captured sample polynucleotide comprises synthesizing a complementary strand from the array probe using the captured sample polynucleotide as a template.

In certain example embodiments, copying the barcoded recognition molecule barcode comprises synthesizing a complementary strand from the array probe using the captured barcoded recognition molecule barcode as a template.

In certain example embodiments, the method further comprises specifically binding a concatemer to a copied barcode recognition molecule(s), copied sample polynucleotide(s), or both.

In certain example embodiments, the sample, the barcoded recognition molecule, captured barcoded recognition molecule barcode, captured sample polynucleotide or a combination thereof is/are removed prior to detecting the copied barcoded recognition molecule barcode, copied polynucleotide, or both.

In certain example embodiments, detecting the copied barcoded recognition molecule barcode, the copied sample polynucleotide, or both comprises specifically binding one or more detectable probes to the copied barcoded recognition molecule barcode, the copied sample polynucleotide, concatemer, or a combination thereof.

In certain example embodiments, detecting the copied barcoded recognition molecule barcode comprises specifically binding a first detectable probe to a first copied barcoded recognition molecule barcode corresponding to a first target and optionally specifically binding a second detectable probe to a second copied barcoded recognition molecule barcode corresponding to a second target.

In certain example embodiments, detecting the copied sample polynucleotide comprises specifically binding a first detectable probe to a first copied sample polynucleotide corresponding to a first sample polynucleotide and optionally specifically binding a second detectable probe to a second copied sample polynucleotide corresponding to a second sample polynucleotide.

In certain example embodiments, the first detectable probe specifically bound to the first copied barcoded recognition molecule barcode is removed prior to specifically binding the second detectable probe to the second copied barcoded recognition molecule barcode.

In certain example embodiments, specifically binding a first detectable probe to a first copied barcoded recognition molecule barcode and specifically binding a second detectable probe to a second copied barcoded recognition molecule barcode occurs simultaneously.

In certain example embodiments, the first detectable probe specifically bound to the first copied sample polynucleotide is removed prior to specifically binding the second detectable probe to the second copied sample polynucleotide.

In certain example embodiments, specifically binding a first detectable probe to a first copied sample polynucleotide and specifically binding a second detectable probe to a second copied sample polynucleotide occurs simultaneously.

In certain example embodiments, the first detectable probe comprises a first label and the second detectable probe comprises a second label and wherein the first label and the second label are different.

In certain example embodiments, the first detectable probe comprises a first label and the second detectable probe comprises a second label and wherein the first label and the second label are the same.

In certain example embodiments, the detectable label on the optionally present non-barcoded recognition molecule is different than a first label or a second label present on the first or second detectable probes when present.

In certain example embodiments, the method further comprises preparing a cDNA library from the captured sample polynucleotide.

In certain example embodiments, preparing a cDNA library comprises preparing a cDNA library PCR product, or both from the copied sample polynucleotide.

In certain example embodiments, the method further comprises preparing a cDNA library, PCR product, or both from the copied barcoded recognition molecule barcode.

In certain example embodiments, the copied sample polynucleotides and array probes are released from the fixed addressable array prior to generating a cDNA library, PCR product, or both.

In certain example embodiments, the copied barcoded recognition molecule barcodes and array probes are released from the fixed addressable array prior to generating a cDNA library, PCR product, or both.

In certain example embodiments, the method further comprises sequencing the cDNA library, PCR product, or both.

In certain example embodiments, the method further comprises correlating each of the cDNA molecules in the cDNA library, each PCR product, or both to a position in the sample on the fixed addressable array.

In certain example embodiments, the method further comprises assigning a cell type, cell subtype, cell state, or any combination thereof to the plurality of cells in the sample, the assigning comprising detecting differential expression of the cDNA molecules, PCR product(s), or both, to generate a gene and/or protein signature and identifying cell type, cell subtype, cell state, or any combination thereof based on the gene signature at positions in the sample.

In certain example embodiments, the barcoded recognition molecule, the non-barcoded recognition molecule, or both comprise a polynucleotide guided nucleic acid targeting system or molecule thereof, an antibody or fragment thereof, an aptamer, or a combination thereof.

In certain example embodiments, the polynucleotide guided nucleic acid targeting system or molecule thereof is a CRISPR-Cas system or a combination thereof.

In certain example embodiments, the method further comprises sequencing the operatively coupled material.

In certain example embodiments, the method further comprises sequencing the directly captured material, indirectly captured material, or both.

In certain example embodiments, the fixed addressable array further comprises a substrate, wherein the plurality of array probes of the fixed addressable array are coupled to the substrate.

In certain example embodiments, the substrate comprises a solid substrate, a semi-solid substrate, a liquid substrate, or a hydrogel.

In certain example embodiments, the substrate comprises a polymer, wherein the polymer optionally forms a layer on a surface of the substrate, and wherein the plurality of array probes are coupled to the polymer.

In certain example embodiments, the substrate comprises a plurality of wells, wherein the plurality of wells is optionally organized in an array.

In certain example embodiments, the substrate comprises an optically transparent material.

In certain example embodiments, the method further comprises releasing the plurality of array probes from the substrate.

In certain example embodiments, releasing comprises cleaving a cleavable linker on each array probe of the plurality of array probes.

In certain example embodiments, the cleavable linker is a restriction enzyme site, and releasing the plurality of array probes comprises cleaving the restriction enzyme site with a restriction enzyme specific to the restriction site.

In certain example embodiments, the method further comprises depositing one or more CRISPR-Cas systems or components thereof onto the substrate.

In certain example embodiments, the one or more CRISPR-Cas systems or components thereof are deposited at each x,y position defined by the fixed addressable array.

In certain example embodiments, each of the one or more CRISPR-Cas systems or components thereof comprises a guide sequence, wherein each guide sequence is coupled an array probe in the plurality of array probes.

In certain example embodiments, the guide sequence is coupled to the spatial barcode of the array probe.

In certain example embodiments, the method further comprises delivering one or more CRISPR-Cas systems or components thereof to the sample prior to depositing the sample on the fixed addressable array.

In certain example embodiments, the fixed addressable array is contained in a droplet.

In certain example embodiments, the sample is a tissue sample.

In certain example embodiments, detecting the copied sample polynucleotides, the labeled dNTPs, the copied barcoded recognition molecule barcodes, the sample, or any combination thereof comprises in-situ sequencing, laser scanning, fluorescent microscopy, DNA microscopy, FISH, smFISH, in situ PCR, or any combination thereof.

In certain example embodiments, the method further comprises detecting the labeled recognition molecule.

In certain example embodiments, the labeled recognition molecule comprises a polynucleotide guided nucleic acid targeting system or molecule thereof, an antibody or fragment thereof, an aptamer, or a combination thereof.

In certain example embodiments, the polynucleotide guided nucleic acid targeting system or molecule thereof is a CRISPR-Cas system or a combination thereof.

In certain example embodiments, detecting comprises imaging the labeled recognition molecule.

In certain example embodiments, operatively coupling the material from the sample to the bead array comprises depositing the sample comprising a plurality of cells on the decoded array and allowing at least some of the plurality of conductive beads to each couple to one or more of the plurality of cells.

In certain example embodiments, each conductive bead is a magnetic bead.

In certain example embodiments, the spatial barcode is color coded or quenched.

In certain example embodiments, the target molecule of at least one of the bead probes is captured by a capture probe of an array probe of the fixed addressable array.

In certain example embodiments, the decoded bead array was decoded by sequential hybridization and detection of the color coded or quenched spatial barcodes.

In certain example embodiments, the method further comprises calculating for background, calculating for errors, or both using the quenched spatial barcodes.

In certain example embodiments, decoding the bead array further comprises in-situ sequencing, laser scanning, DNA microscopy, fluorescent microscopy, laser scanning, FISH, smFISH, in-situ PCR, or a combination thereof.

In certain example embodiments, in-situ sequence comprises Illumina sequencing or Nanopore sequencing.

In certain example embodiments, one or more array probes comprises an oligonucleotide sequence, wherein one or more bead probes comprises an oligonucleotide sequence, or both.

In certain example embodiments, the capture molecule(s), the target molecule(s), or both comprises a Tn5 sequence, a 16S sequence, a poly(d)T sequence, a poly(d)A, sequence a random hexamer sequence, a trypsin molecule, an antibody, an aptamer, a Protein Epitope Signature Tag (PrEST) sequence, a DNA sequence or structural variation, or a combination thereof.

In certain example embodiments, the DNA sequence or structural variation is a single nucleotide polymorphism or a copy number variation.

In certain example embodiments, one or more array probes, one or more bead probes, or both further comprise one or more of a unique molecular identifier (UMI), an adapter sequence, and a primer sequence.

In certain example embodiments, the method further comprises ablating a single layer of the sample and performing the step of operatively coupling material from the sample in a second layer of the sample.

In certain example embodiments, any one or more of the steps is automated.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1G—High-density spatial transcriptomics (HDST). (FIG. 1A) HDST workflow. Barcoded beads are randomly deposited into single wells and the barcode carries an oligonucleotide sequence encoding a (x,y) position in each individual array present on the silicon wafer. Frozen tissue sections are placed on the array surface and H&E stained. Morphology is recorded at the same time of recording of the relative positions of each bead (x,y) to the tissue section. mRNA is captured on the oligonucleotide capture sequenced and cDNA made. Now, the spatial oligonucleotide sequence is covalently linked to the mRNA information for each cell in the tissue. Standard pair-end sequencing libraries are made, spatial oligonucleotides demultiplexed and the whole tissue section profiled with high-density spatial transcriptomics. (FIG. 1B) HDST H&E image of a main olfactory bulb and HDST (x,y) barcodes annotated into 9 different morphological areas (RMS; E; GCL-I, GCL-E, IPL, M/C; EPL; GL and ONL). (FIG. 1C) Differentially expressed and upregulated gene patterns detected between different morphological layers in HDST; (FIG. 1D) Labeling of morphological layers. HDST H&E image of a MOB and matching HDST (x,y) barcodes annotated into nine morphological areas. (FIG. 1E) Layer-specific DE patterns in HDST. Shown is the summed normalized expression of positively enriched signature genes significantly (FDR <0.1, two-sided t-test) associated with each layer as annotated in FIG. 1D. FIG. 1F-1G, Nuclei segmentation and binning of HDST as in FIG. 1D. (FIG. 1F) Segmented nuclei (sn-like) and lightly binned (sc-like) spatial barcodes assigned (black) to each of two cell types as in FIG. 1D. (FIG. 1G) Enrichment of sn- and sclike spatial barcodes with assigned cell types (columns) to morphological layers (rows) as in 1D. Color bar represents −log 10 (P value) (one-sided Fisher's exact test, Bonferroni adjusted, P<0.01) and gray tiles are nonsignificant values. OBNBL1, olfactory neuroblasts; OBINH1-3, inhibitory neurons; EPMB and EPEN, astroependymal cells; OEC, olfactory ensheathing cells; VLMC2, vascular cells; SATG2, satellite glia; OBNBL5, GABAergic neuroblasts; OBDOP1, dopaminergic periglomerular neuroblasts; OBNLB2, VGLUT1/2 neuroblasts; SEZ, subependymal zone; ONL, olfactory nerve layer; M/T, mitral layer; IPL internal plexiform layer; GCL-E, GCL-I and GCL-D, granular layers; GL, glomerular layer; EPL, external plexiform layer.

FIG. 2A-2F—High-density spatial transcriptomics (HDST) array performance. (FIG. 2A) Average decoding efficiency and barcode redundancy for all generated slides (n=30) as well as average spatial barcode demultiplexing after sequencing (n=3) (FIG. 2B) Average sequencing depth and library saturation (n=3) (FIG. 2C) Total number of barcodes, genes and UMI counts demultiplexed, mapped and filtered under the tissue boundaries for replicate libraries (n=3). (FIG. 2D) Density plot depicting frequencies of UMI counts per spatial barcode found in each replicate library. (FIG. 2E) Total number of barcodes, genes and UMI counts demultiplexed, mapped and filtered outside the tissue boundaries for replicate libraries (n=3). (FIG. 2F) Heatmap of total counts per spatial barcode for all three replicates.

FIG. 3A-3B—Summary statistics for comparisons to bulk RNA-seq dataset. (FIG. 3A) Correlation of average gene expression between HDST replicates and bulk RNA-seq; (FIG. 3B) Venn diagram showing numbers of shared or present genes for all three HDST replicates and bulk RNA-seq dataset.

FIG. 4A-4D—Cell typing in high-density spatial transcriptomics (HDST). (FIG. 4A) Combinatorial approach for assigning cell types to spatial (x,y) barcoded transcriptomes. Top panel represents UMI-filtered transcript counts for cell types present in the Zeisel et al single cell RNA-seq dataset. Right panel represents UMI-filtered transcript counts present for two example barcode in HDST. The spatial cell typing panel represent the testing results for each HDST barcode dataset against each cell type, where n (number of total counts) is shared with the single-cell dataset. Likelihood scores are calculated for each combination and highest score indicates the cell type assignment to the spatial (x,y) barcode dataset. (FIG. 4B) Top: Average normalized likelihood scores for all cell types imputed onto all spatial (x,y) barcoded transcriptomes. with three distinct cell populations assigned to their spatial (x,y) coordinates (OBINH: inhibitory neurons; OBNLB: neuroblasts; OBDOP: dopaminergic neurons; shown in red with grey presenting all (x,y) coordinates). Bottom: Fisher's test showing cell type populations enriched per annotated morphological layer. (FIG. 4C) Average normalized likelihood scores for all cell types in a downsampled and thinned HDST (38×; left) and standard ST dataset (ST; right). (FIG. 4D) Percentages of (x,y) barcodes assigned to different neuronal populations.

FIG. 5A-5B—Accessing data convolution with binning. (FIG. 5A) Density plots depicting frequencies of observations for normalized cell type likelihoods in binned (5×) and HDST data (FIG. 5B) Histogram of number of different cell types found per bin as compared to HDST. Different bin sizes were used: 38×, 20×, 10×, and 5×.

FIG. 6A-6C—Spatial morphology and differential expression. (FIG. 6A) Morphological annotation of the standard ST dataset into nine layers. (FIG. 6B) Automatic expression histology patterns detected in the standard ST dataset. (FIG. 6C) Overlay of automatic expression histology patterns present in (FIG. 6B) in the HDST dataset.

FIG. 7A-7B—Spatially upregulated genes per morphological layer. (FIG. 7A) Downregulated differentially expressed gene between morphological layers in HDST. (FIG. 7B) Scaled gene expression for all differentially expressed genes (columns) per morphological layer (rows).

FIG. 8A-8B—Validation for upregulated HDST genes using the Allen Brain Atlas (ABA) data. (FIG. 8A) Fisher's test showing enrichment in HDST layers compared to corresponding ABA layer data. (FIG. 8B) Top panel: ABA ISH data for top genes per layer. Bottom panel: Heatmaps showing expression of all genes that overlap in expression per layer in both HDST and ABA.

FIG. 9—Schematic overview of new learning method, insi2vec, allows defining a cell by both intrinsic and extrinsic features.

FIG. 10—Schematic of model sc2st that allows extension to full transcriptome, taking single cell profiles from scRNA-seq and use to expand the in situ data.

FIG. 11—Images of Insi2vec application to pyramidal neurons L6 showing identification of subsets that cannot be resolved otherwise.

FIG. 12 shows results of application of insi2vec embedding to cluster cells and discovery of distinct subsets of immune and malignant cells by intrinsic and spatial features.

FIG. 13 shows images showing application of insi2vec model generalizes across patients.

FIGS. 14A-14C—FIG. 14A is an example spatial gel matrix (1b) from osmFISH for a gene (Syt6); FIG. 14B is a scatterplot of the spatial gene expression for Syt6 in the somatosensory cortex; FIG. 14C is a reconstructed image using a linear radial basis function interpolation.

FIG. 15—An example image of a cell and its neighbors with the full image having 33 channels.

FIG. 16—Depicts spatially relevant subsets of cells using traditional clustering approaches using learned spatio-transcriptomic embeddings.

FIG. 17A-17G—Exemplary Melanoma Tumor evaluation using insi2vec. (FIG. 17A) Clustering in-situ data by treating it as a scRNAseq dataset. (FIG. 17B) Expression pattern of the example gene (CD8a) in-situ. (FIG. 17C) Visualization after running the spatio-transcriptomic clustering and visualizing the data using the learned embeddings. (FIG. 17D) Overlaying the clusters found in non-spatial clustering of FIG. 17A onto the spatio-transcriptomic embeddings of. (FIG. 17E) Spatio-Transcriptomic embedding reveals distinct flavors of CD8T cells in the melanoma tumor in-situ data. (FIG. 17F) cluster 1 from non-spatio-transcriptomic clusters (the CD8 T cell cluster) viewed from the spatio-transcriptomic cluster; Applicants notice three distinct flavors of CD8 T Cells in the form of clusters 2,7, and 8. These new CD8 T cell subsets are defined by their transcriptomes and their neighborhoods in this non-canonical melanoma tumor sample. (FIG. 17G) Individual CD8 T Cell Subsets along with malignant cells (Malignant Cells are Red as represented by greyscale).

FIG. 18A-18C—HDST distinguishes cell types and niches in a breast cancer resection. (FIG. 18A) Labeling of morphological layers. HDST H&E image (left) of a breast cancer section and matching HDST (x,y) barcodes annotated into 13 morphological areas (right, color code). (FIG. 18B) Layer-specific spatial DE patterns in HDST. Summed normalized expression of positively enriched signature genes significantly (FDR <0.1, two-sided t-test) associated with each layer as in FIG. 18A. (FIG. 18C) Cell type assignments by single nuclei as in FIG. 18A. Two enlarged regions (black and red squares) with II&E and color-coded segments, Methodology, supporting data and supplementary material are as described in Vickovic, et al., Nature Methods, DOI:10.1038/s41592-019-0548-y, specifically incorporated herein in its entirety by reference.

FIG. 19A-19H′—Includes results from H&E staining and fluorescent imaging, fluorescent gene activity footprints, and histograms of distances between H&E boundaries and fluorescent prints. (FIGS. 19 and 19A′) H&E image of the cortex region on the mouse brain for manually prepared samples. (FIGS. 19B and 19B′) H&E image of the cortex region on the mouse brain (adjacent section to (FIG. 19A) for ST2.5 samples. (FIGS. 19C and 19C′) Fluorescent gene activity footprints corresponding to FIG. 19A and FIG. 19A′. (FIGS. 19D and 19D′) Fluorescent gene activity footprints corresponding to (FIG. 19B) and (FIG. 19B′). (FIGS. 19E-19F) Histograms of distances between detected H&E cell boundaries and fluorescent prints for manual and ST2.5 preparations. (FIGS. 19G-19G′) H&E and fluorescent print for the main olfactory bulb of the adult mouse brain. (FIGS. 1911-1911′) H&E and fluorescent print for the MC38-OVA injected cell lines into a preclinical model of colorectal cancer.

FIG. 20A-20C—Characterization of automated processes. (FIG. 20A) Mean fragment length distribution with 68% confidence interval of amplified RNA for automated prepared samples (n_{biological}=3) from three separate robot runs. (FIG. 20B) qPCR generated Cq values for automated prepared libraries (n_{biological}=3) from three separate robot runs. Statistical significance (t-test) is displayed. (FIG. 20C) qPCR generated Cq values for automated prepared libraries in four (n_{biological}=12), six (n_{biological}=18) and twelve (n_{biological}=36) columns in three rows. Statistical significance (t-test) is displayed. Cq values for both FIGS. 20B and 20C were measured at Fluorescent unit 10,000. 0.05<p<=1 (ns), 0.001<p<=0.01 (**), p<=0.0001 (****).

FIG. 21A-21B SpoTter-based array and tissue detection (FIG. 21A) The RGB tissue H&E stained image is the input file to the approach. First the RGB image is split into 3 color channels and circular features are detected. Those features that potentially fit a grid pattern (33×35 matrix) are used for the initial fit. Then circular features outside the grid are removed and the process of grid fitting repeated until a perfect 33×35 matrix is adjusted and positioned. Then the tissue is detected and grid spots under the tissue easily selected. (FIG. 21B) SpoTteR performance for three different tissue types.

FIG. 22A-22C—SpoTteR performance. (FIG. 22A) False negative and positive ST barcode (x,y) positions using SpoTteR (blue cross) or ST Detector (black circle) as compared to the manually curated positions (filled red circle) for a mouse colon sample. (FIG. 22B) Total false negative and positive rates per processed tissue type. (FIG. 22C) Processing speed (given as 1/time s⁻¹) for three tested processing approaches.

FIG. 23A-23B (FIG. 23A) Mean fragment length distribution of DNA molecules prepared for sequencing with 68% confidence interval for automated prepared libraries (n_{biological}=3 for conditions ‘STD 1h’, ‘STD 3h’, ‘STD+5× adapt 3h’ and n_{biological}=2 for condition ‘STD+5× adapt 1h’) using conditions stated in the legend. Diamonds represent the average fragment lengths. (FIG. 23B) Quantitative concentrations (Cq) values for automated prepared libraries (n_{biological}=3, n_{technical}=3) using conditions stated on the x axis. Cq values were measured at fluorescent unit 10000. Statistical significance using T-test is displayed. Conditions: ‘STD 1h’:‘1× adapter concentrations, 1 hour ligation’, ‘STD 3h’: ‘1× adapter concentrations, 3 hours ligation’, ‘STD+5× adapt 1h’: ‘5× adapter concentrations, 1 hour ligation’, ‘STD+5× adapt 3h’:‘5× adapter concentrations, 3 hour ligation’. 0.05<p<=1 (ns), 0.001<p<=0.01 (**), 0.0001<p<=0.001 (***), p<=0.0001 (****).

FIG. 24A-24E—(FIG. 24A) Number of expressed genes for ST2.5 and manually prepared libraries and their intersection. Gene count has been adjusted for sequencing depth (Methods). (FIG. 24B) qPCR generated Cq values for ST2.5 and manual prepared libraries (n_{biological}=3). Statistical significance (t-test) is displayed. (FIG. 24C) Correlation of the pseudo-bulk and normalized gene expressions between ST2.5 and manual prepared libraries (n_{biological}=3). Denoted is the Pearsons's correlation coefficients between replicates. Grey line represents the linear regression line between the replicates. (FIG. 24D) Proportion of unique molecules (adjusted for number of annotated reads as described in Methods) per annotated region in ST2.5 (n_{biological}=3) and manually prepared libraries (n_{biological}=3). (FIG. 24E) Correlation of the pseudo-bulk and normalized gene expressions between ST2.5 and ST for 3 annotated regions: Granula Cell Layer Deep (GCL-D), Glomerular Layer (GL) and Olfactory Nerve Layer (ONL). Denoted is the Pearsons's correlation coefficient between the replicates. Grey line represents the linear regression line between replicates. Gene count has been adjusted for sequencing depth (Methods).

FIG. 25A-25C—(FIG. 25A) Shared genes with ABA (Allen Brain Atlas) in all annotated regions: GL, GR, MI and OPL in ST2.5 and manual prepared libraries. Grey scale denotes significant p-values (p<0.05, Fisher's exact test, one sided, multiple testing corrected using Benjamini/Hochberg). (FIG. 25B) Spatial gene expression of expressed DE genes in region GL, GR, IPL, MI and OPL in ST2.5 (i), with corresponding gene expression (ii) and ISH image (iii) from ABA. (FIG. 25C) Spatial gene expression of expressed DE genes with ST2.5 which could not be found in the ST reference (i) in region GL, GR, IPL, MI and OPL, with corresponding gene expression (ii) and ISH image (iii) from ABA. GL (Glomerular Layer), GR (Granule Cell Layer), MI (Mitral Layer), IPL (Internal Plexiform layer) and OPL (External Plexiform Layer).

FIG. 26—Shows a plurality of array probes forming a fixed array with on a substrate. A tissue can be placed on the fixed array. The tissue can be stained using any suitable stain, such as H&E or other stain (e.g., a fluorescent stain) and imaged.

FIG. 27—Antibodies having an antibody barcode (e.g., Total Seq Antibodies) can be allowed to specifically bind epitopes in the tissue.

FIG. 28—The bound antibodies can be stained an imaged using any suitable stain or dye or detectable signal reaction.

FIG. 29—The antibody barcodes can bind the probes in the fixed array.

FIG. 30—After binding of antibodies having antibody barcodes to the correct epitopes, the antibody barcodes can be copied via e.g., second strand synthesis.

FIG. 31—After copying the antibody barcodes, the tissue and antibodies can be removed.

FIGS. 32A-32B—The copied antibody barcodes can be detected using (FIG. 32A) imaging probes comprising a detectable label and recognition molecule that can specifically bind or otherwise interact with a copied antibody barcode and/or (FIG. 32B) a probe capable of binding a specific antibody barcode followed by detection of the bound copied antibody barcode with a detection molecule specific to the probe or probe:copied antibody barcode complex.

FIG. 33—Where different antibody codes are detected in series, imaging probes (or other detection molecules or complexes) can be removed between detection of each antibody barcode. The array can be imaged to confirm removal of the probes before detecting the next copied antibody barcode.

FIG. 34—After binding of the copied antibody barcode(s) with a probe and/or detectable label as described in connection with e.g., FIGS. 32A-32B, the array can be imaged. In some embodiments, this can be done in series (i.e., one antibody barcode at a time) as shown in FIG. 34.

FIG. 35—Multiplexing of antibody barcode detection. Different probes with different labels can be used simultaneously to detect/image multiple antibody barcodes (and thus multiple epitopes). In some embodiments, the tissue can be imaged to detect imaging probes and thus the copied antibody barcodes. Using different probes with different labels can allow for simultaneous detection (simultaneous multiplexing) of different copied antibody barcodes.

FIG. 36—Combined spatial proteomics and transcriptomics on ST arrays. As in FIGS. 26-35, protein information via antibodies through copied antibody barcodes can be spatially captured. In some embodiments shown here, transcription information can also be simultaneously captured. Labeled dNTPs can be incorporated in the copied barcodes during second strand synthesis and in situ cDNA synthesis.

FIG. 37—Copied antibody barcodes can be detected and imaged as described e.g., in FIGS. 32A-35. Incorporated labeled dNTPs can likewise be imaged.

FIG. 38—After imaging as described in FIG. 36, cDNA and copied antibody barcodes can be cleaved from the array and be used to generate sequencing libraries. The barcoding scheme can facilitate spatial resolution of the sequenced transcripts.

FIGS. 39-40—In some methods of combined spatial proteomics and transcriptomics on ST arrays, antibodies not having an antibody barcode can be allowed to bind to their epitopes in a tissue on the fixed array. Antibodies can be detected (e.g., via a label coupled to the antibody or by a labeled secondary antibody) using any suitable imaging technique

FIG. 41—In some embodiments, antibody binding and detection can be multiplexed e.g., via the use of different labels and/or different primary antibody types (e.g., IgG, IgM, IgA, etc. and/or sources (rabbit, goat, chicken etc.).

FIG. 42—cDNA can be generated in situ and labeled dNTPs can be incorporated during strand synthesis.

FIG. 43—The cDNA can be cleaved from the array and be used to generate a sequencing library.

FIGS. 44-45—Show a general scheme for a bead array. Magnetic beads having probes attached can be captured into an array using a magnet or other electromagnetic field underneath a fixed array of probes on a substrate. The probes on the magnetic beads can have spatial barcodes that couple with different dyes (such as red and green as represented in greyscale) or none or a quencher (dark). Dark beads can be used for error and/or background correction. The bead array can be decoded by sequential hybridization against the different spatial barcodes on the beads.

FIG. 46—Shows an image of a bead array. Left image shows a mix of green, red, and dark beads. Right image shows a mix of green and red (as represented in greyscale) only. Dark is used to calculate errors and background.

FIG. 47—The bead arrays can be hybridized, stripped, and imaged repeatedly. The cycle of hybridizing, stripping, and imaging for an entire slide filled with arrays is about 25 minutes. In some embodiments, multiple slides (e.g., 2 to about 48 with about 1-96 spatial arrays per slide) can be processed at once. As long as the magnet or other electromagnetic field is present beneath the substrate, the beads do not substantially move during the process.

FIG. 48—Tissue can be placed on top of the decoded arrays, the magnet or other electromagnetic field can be removed or otherwise modified to release unbound/unstuck beads, and the tissue can be stained. The beads can stick to the cells. The beads stick only to the closest cells/intact surfaces. The beads surrounding those stuck to the cells are washed away. Beads that are washed away can be captured and reused. The ability to capture and reuse the beads provides a cost and materials saving advantage not realized by other techniques. Sectioning artifacts can evidence that the beads stick only to the closest cells/intact surface.

FIG. 49—Various embodiments of substrates that the array probes can be coupled to.

FIG. 50—General schematic of copying spatial antibody information into z=1 space and using imaging as a read-out.

FIG. 51—Results on mouse spleen after performing the general scheme shown in FIG. 50 using Ab (F4/80 total-seq antibodies). Fluorescence (indicated by greyscale representative of green fluorescence) in images 2 and 3 indicate signal above threshold. The positive control shows regular antibody staining in the tissue using the same clone but with a Cy5-coupled antibody (Ab) as read out on an Epi scope. The pulp should stain red (represented in FIG. 51 in grayscale) based on the Cy5 label.

FIG. 52—Iteration of images 2 and 3 (see e.g., FIG. 50) with the same fluorescent labeled-antibody-barcode. The surface appeared very stable, and results were reproducible between stripping cycles. Results shown are without any signal amplification.

FIG. 53—Reaction conditions were modified to improve signal to noise ratio to 4 to 1.

FIG. 54—A control that can demonstrate that antibody reaction conditions still generate an mRNA/cDNA print on the array surface. mRNA quality can be checked without adding any antibodies but with using the same reaction conditions.

FIG. 55—General scheme of combining spatial proteomics with transcriptomics on ST arrays.

FIG. 56—Results after imaging for copied antibody barcodes (same as general scheme shown in FIG. 50) and incorporated labeled dNTPs (Cy3 labeled dCTP). Fluorescence (indicated by greyscale representative of green fluorescence) in images 2 and 3 indicate signal above threshold. The same principle of detection was applied here as in FIGS. 50-54. In image 3, Cy3 signal (represented in greyscale) is for copied mRNA and Ab barcode as the barcode contains some cytosines (Cs).

FIG. 57—cDNA probes can be cleaved from the surface of the array for library creation. Imaging can check for how many probes are available after cDNA synthesis and cleaving of probes from the surface. A blank (dark) image indicates (e.g., image 1) that cDNA synthesis and cleavage of probes was successful.

FIG. 58—Validation of PCR products (antibody barcode information) and final ST2.5 cDNA libraries (mRNA information).

FIG. 59—Combined spatial proteomics and transcriptomics POC sequencing results from combined spatial proteomics and transcriptomics on ST arrays (see general scheme in FIG. 50).

FIG. 60—Combined spatial proteomics and transcriptomics POC results for CD4, CD8a and CD19. Bottom images denote fluorescent staining images.

FIG. 61—Combined spatial proteomics and transcriptomics POC results (replicate section based on 1M reads).

FIG. 62—General scheme for another embodiment of a combined spatial proteomics and transcriptomics using antibodies conjugated with fluorophores or other detectable label. This is combined with ST.

FIG. 63—Combined spatial proteomics and transcriptomics POC results in mouse cortex after performing the general scheme set forth in FIG. 62. mRNA signal appeared very even throughout the tissue as indicated by the Cy3 signal. At this point, the Cy3 signal from mRNA should be observed everywhere throughout the tissue. Some loss of mRNA integrity was observed with each successive cycle.

FIG. 64—Combined spatial proteomics and transcriptomics on spatial transcriptomic arrays showing POC spatial sequencing results. These results in the mouse cortex (which applied the same sample in each, both prepped ST2.5) can confirm that the system works with fluorescent labels and produces even coverage under the tissue.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

Diagnosis is commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). Identifying a disease state, disease progression, or other abnormal condition, based upon symptoms, signs, and other physiological and anatomical parameters are also encompassed in diagnosis. In certain instances, diagnosis comprises detecting a gene expression profile of a sample, host tissue, cell or cell subpopulation.

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide advancements in spatial transcriptomics (ST) technology, providing for high-density spatial transcriptomics (HDST), in particular in-situ spatial profiling. In particular, the compositions, methods, and techniques described herein couple spatial proteomics with spatial transcriptomics for a spatial multiomic analysis. With techniques that allow for higher resolution and using multiple molecular capture species, cell and/or tissue dynamics can be further interrogated and understood. The disclosed spatial method is a comprehensive tool for massive and combinatorial processing in systems biology. It creates a thorough, collected synopsis of molecules present in a cell (whether eukaryotes or prokaryotic) at maximum spatial and temporal granularity. With this approach, Applicants can simultaneously deconvolve biological processes and validate inter- and intracellular interactions at a targetable protein level. The resulting changes can be coupled to developmental and spatial trajectories with the possibility to tease out immediate and distal environmental impact to cells present in an organ.

### Methods of Spatial Transcriptomics and Spatial Proteomics

Methods of spatiotemporal processing of a sample of a plurality of cells are provided. In embodiments, the method includes the steps of depositing a plurality of spatial barcodes on a solid substrate, the spatial barcodes further comprising a capture molecule; depositing the sample of the plurality of cells on the surface of the solid substrate; and capturing material from one or more cells of the plurality of cells with the capture molecule of the spatial barcode, thereby linking the capture material from the one or more cells with the spatial barcode.

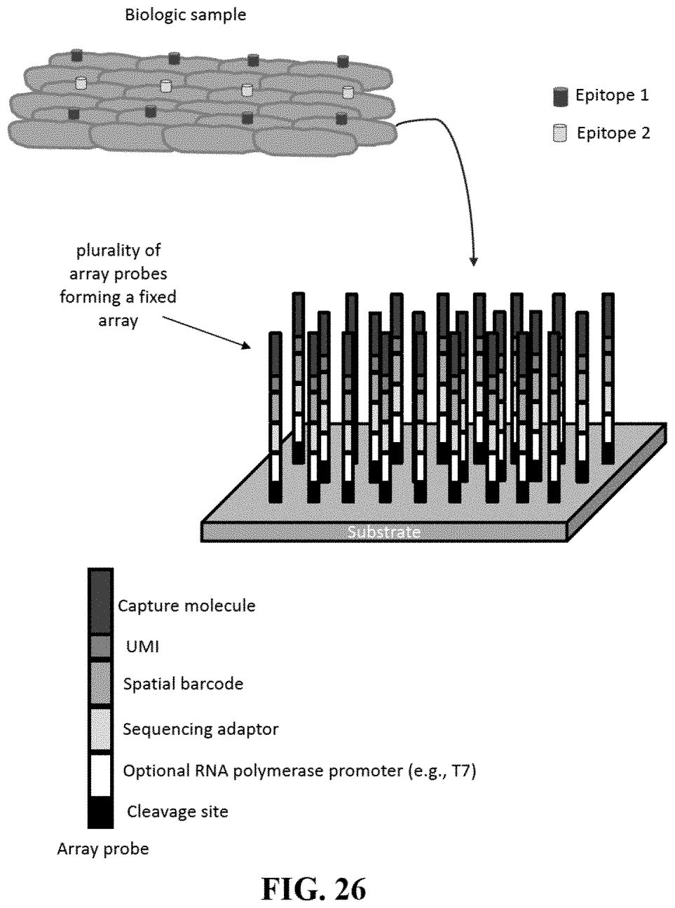

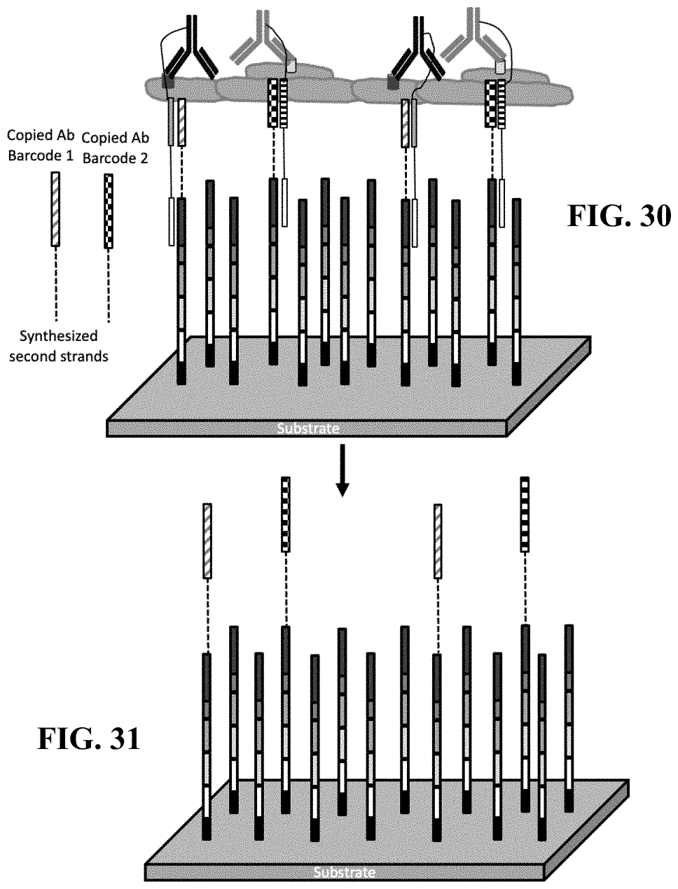

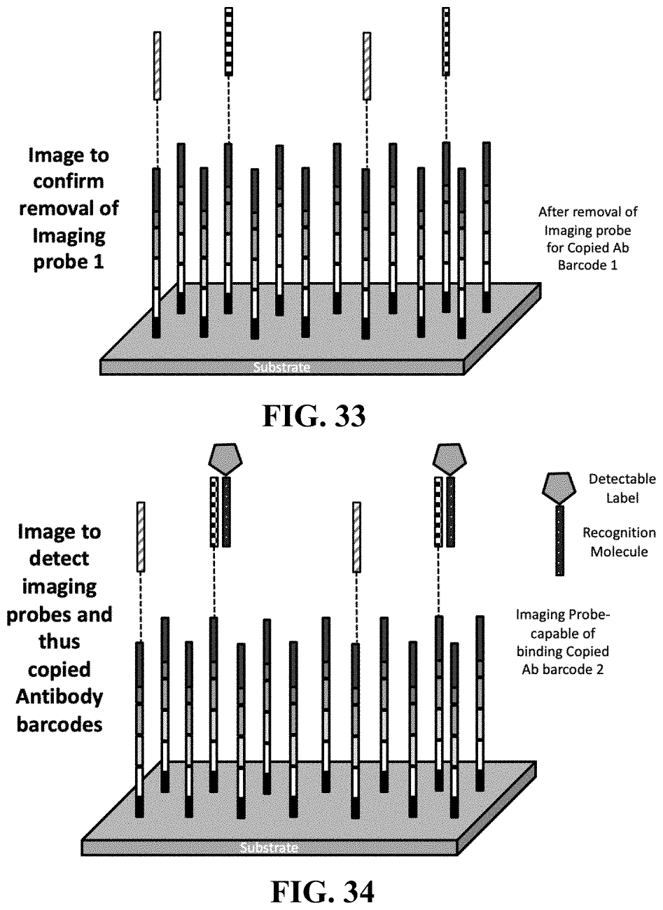

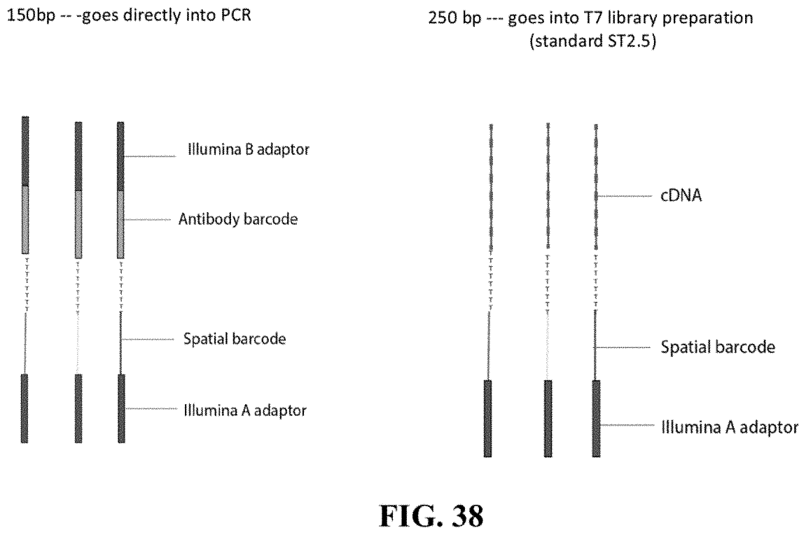

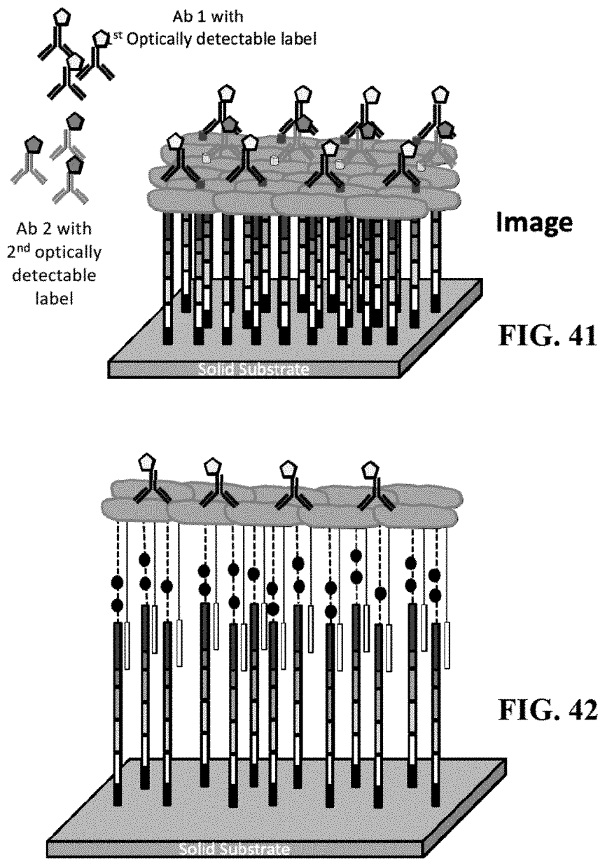

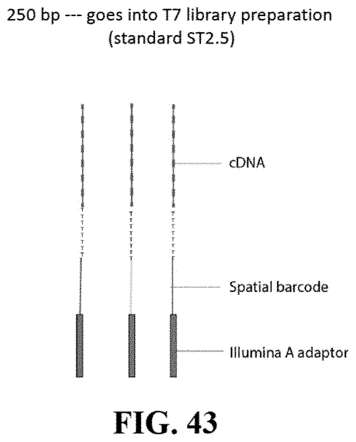

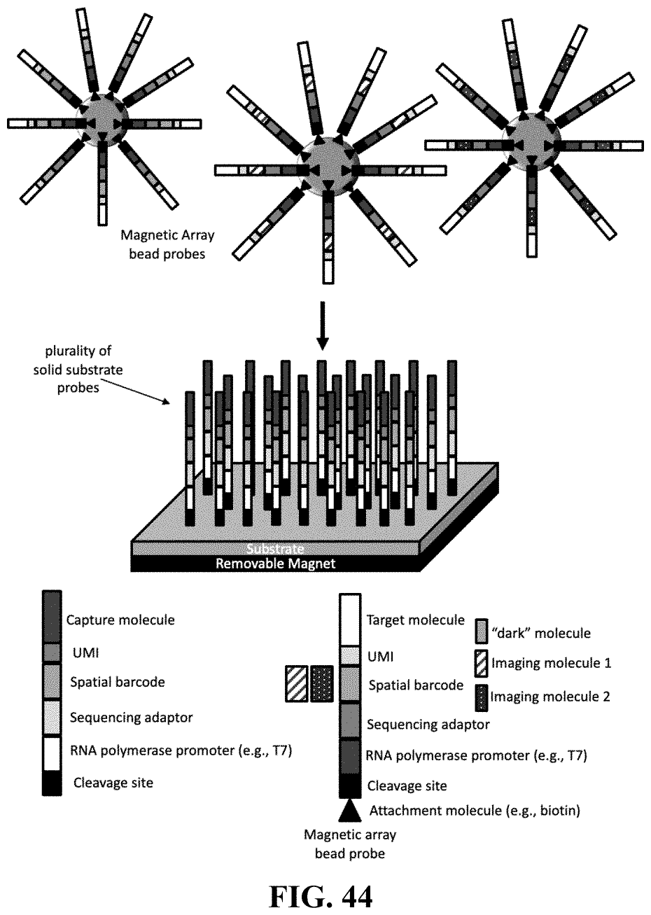

In some embodiments, the spatial transcriptomics (ST) can be combined with spatial proteomics as shown in e.g., FIGS. 26-48. In certain example embodiments, methods of spatial and/or temporal processing of a sample comprising a plurality of cells includes: a. depositing a sample comprising a plurality of cells on a fixed addressable array (see e.g., FIGS. 26-43 and 50-64) or a decoded bead array (see e.g., FIGS. 44-48), wherein the fixed addressable array comprises: a plurality of array probes, each array probe comprising a capture molecule and a spatial barcode, wherein each spatial barcode defines a unique x,y, position of each array probe in the fixed addressable array; ii. wherein the decoded bead array comprises a plurality of conductive beads comprising a plurality of bead probes, each comprising a target molecule and a spatial barcode, wherein the plurality of conductive beads are transiently fixed in spatial position to a first side of the fixed addressable array by an electromagnetic field applied to a second side of the fixed addressable array and wherein the first side and the second side are opposite sides of the fixed addressable array; and b. operatively coupling material from the sample to the plurality of array probes of the fixed addressable array or the plurality of bead probes of the decoded bead array, thereby linking the operatively coupled material from the sample with an x,y position in the fixed addressable array and/or the decoded bead array.

As used in this context herein, “transiently fixed” refers to a temporary and non-permeant fixation of a molecule or molecules in place. In some embodiments, an outside force is used to maintain the fixation, such as a pressure, attractant, magnetic or electromagnetic filed applied to the substrate, sample, and/or other component of the array, that can react with the bead probes such that they are held in place until the force is removed or modified (such as magnetic field change). Any suitable magnet or electromagnetic field or other force can be used to transiently fix the bead probes in place. In some embodiments, transient fixation can be replaced with permeant attachment. For example, in some embodiments, binding or other attachment occurs between the bead probes and the sample such that when the force is removed (e.g., by taking away the magnet), bead probes that bound or are otherwise attached to the sample remain in place while those that have not become un-fixed and can be subsequently removed (e.g., by washing).

In certain example embodiments, operatively coupling material from the sample comprises: directly and/or indirectly capturing material from the sample by a capture molecule of an array probe that is in spatial proximity to the captured material, thereby linking the captured material from the sample with an x,y position in the fixed addressable array. As used herein, “spatial proximity”, refers to the distance within which binding or another interaction described herein between two molecules, such as a capture molecule of the array probe and a target (e.g., a sample target or recognition molecule), can occur. It will be appreciated that if two molecules are physically too far apart no physical interaction, such as binding can occur. In the context of the present disclosure, it will be appreciated that (in the fixed array) that some of the molecules that can be involved in an interaction (such as a binding interaction) are substantially fixed in place at one or more points during the assay, such that only certain regions of a sample or e.g., recognition molecules bound to a sample, will be in appropriate physical distance from an array probe (i.e., spatial proximity) such that can interact to bind or otherwise interact as described herein. Spatial proximity can vary depending on e.g., size, length, flexibility, sample present between two molecules, etc. between any two molecules that can potentially interact. Spatial proximity can be measured (by detecting an interaction using a suitable method, such as any of those described herein) and/or be calculated by taking into consideration the physical characteristics of specific molecules used and characteristics of the sample (physical size and make up, layers of cells present, etc.). Spatial proximity can be varied by molecule design. For example, to increase spatial proximity, longer linkers and/or more flexible linkers can be used to conjugate a recognition molecule to a recognition barcode that is designed to bind to an array probe. Other modifications will be appreciated by those of skill in the art in view of this description herein. In some embodiments, spatial proximity can range from any number above 0 to 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000 angstroms, fm, pm, nm, or μm.

In certain example embodiments, directly capturing material from the sample comprises capturing a sample polynucleotide by hybridizing the sample polynucleotide to the capture molecule of the array probe that is in spatial proximity to the sample polynucleotide or binding a labeled recognition molecule to a target present in the sample.

In certain example embodiments, the sample polynucleotide is or comprises DNA.

In certain example embodiments, the sample polynucleotide is or comprises RNA.

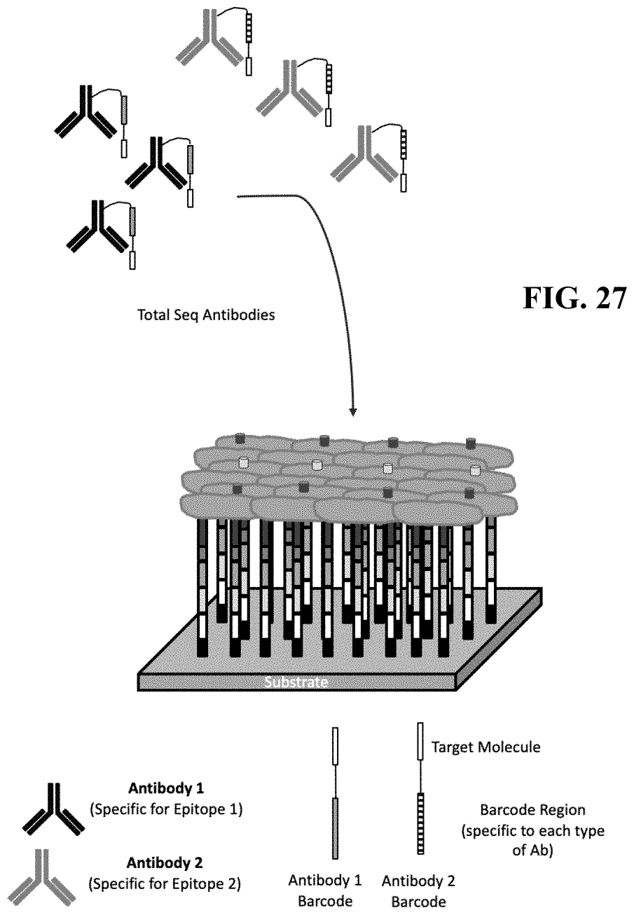

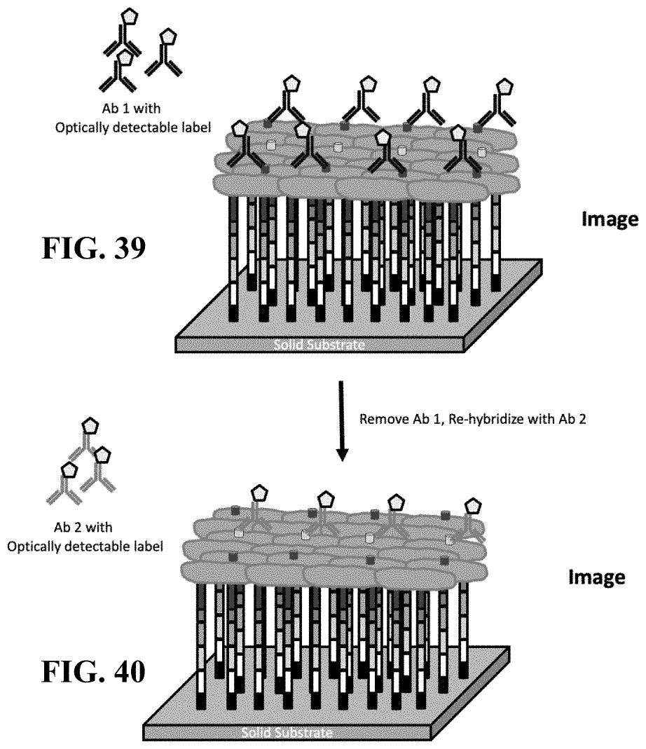

In certain example embodiments, indirectly capturing material from the sample comprises i. specifically binding a barcoded recognition molecule to a target present in the sample (see e.g., FIGS. 27-28, 50, and 55), wherein the barcoded recognition molecule comprises a recognition molecule barcode; ii. optionally, specifically binding a non-barcoded recognition molecule comprising a detectable label to the target present in the sample (see e.g., FIGS. 39-42 and 62); and iii. capturing the barcoded recognition molecule barcode by the capture molecule of the array probe that is in spatial proximity to the target.

In certain example embodiments, the method further comprises copying the captured sample polynucleotide(s), the captured barcoded recognition molecule barcode(s), or both, thereby forming a copied sample polynucleotide(s), a copied barcoded recognition molecule barcode(s), or both (see e.g., FIGS. 29-31, 50, and 55).

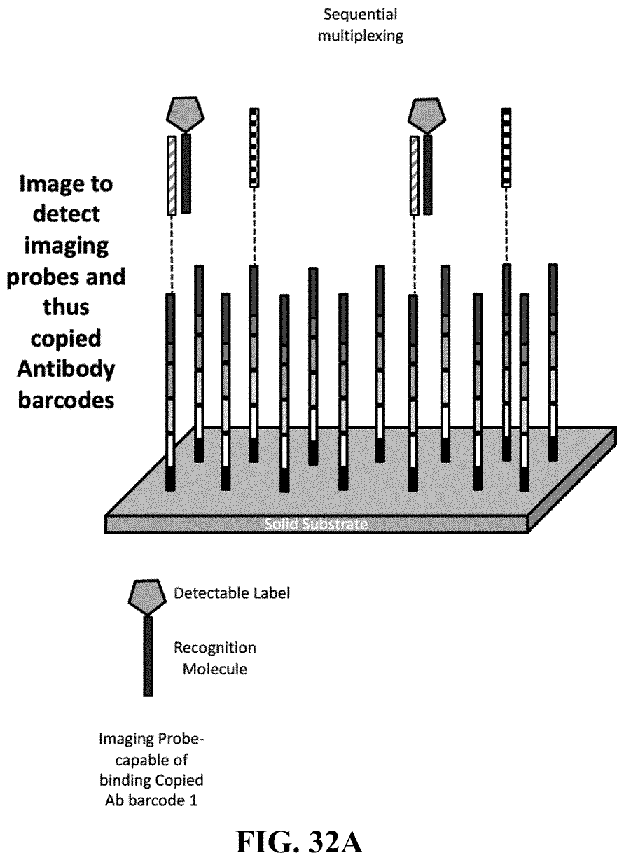

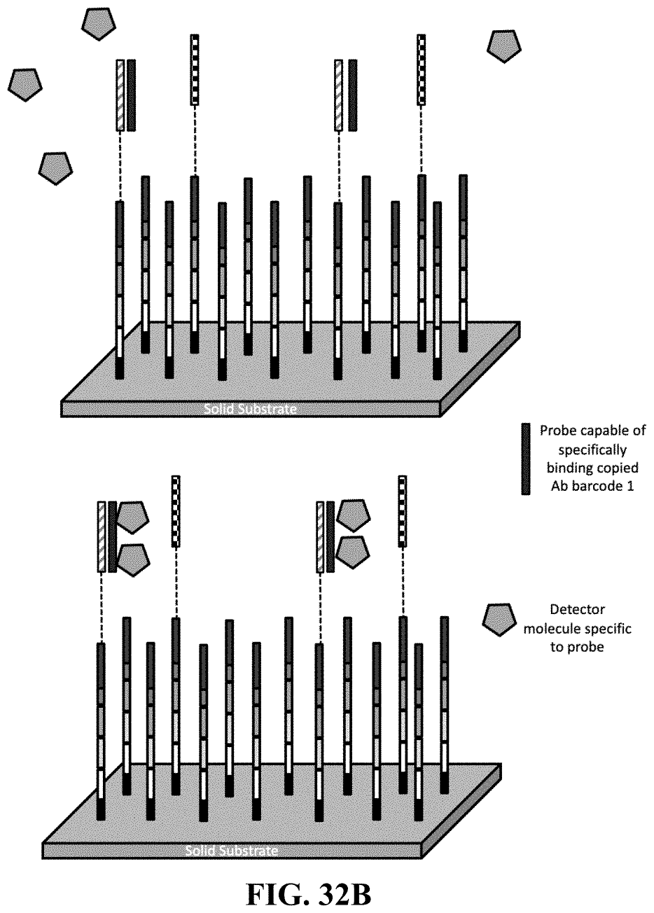

In certain example embodiments, the method further comprises detecting the copied sample polynucleotides, copied barcoded recognition molecule barcodes, or both (see e.g., FIGS. 32A-35, and 50).

In certain example embodiments, detecting comprises imaging the copied sample polynucleotides, the copied barcoded recognition molecule barcodes, or both see e.g., FIGS. 32A-35, and 50).

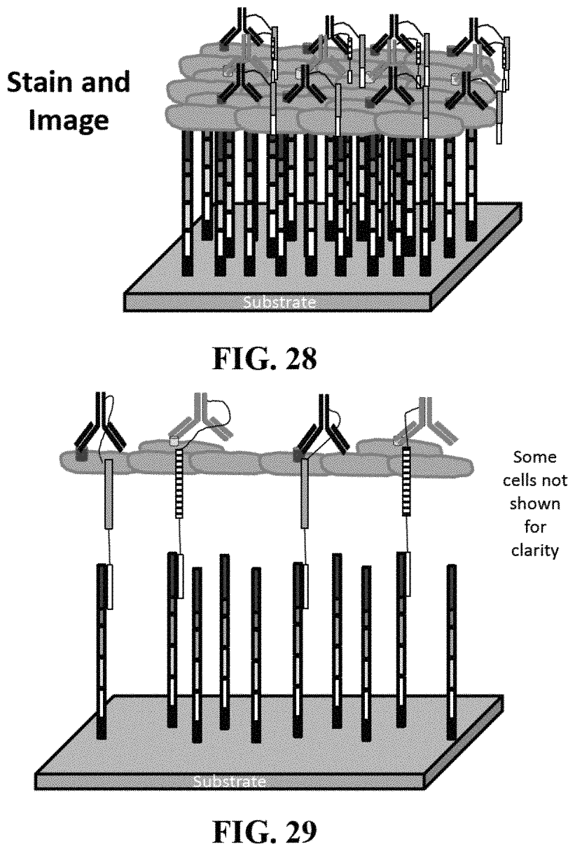

In certain example embodiments, the method further comprises capturing an image of the sample on the fixed addressable array (see e.g., FIGS. 28, 39-41, 50, and 56-57).

In certain example embodiments, the method further comprises annotating regions of the image of the sample, optionally based on morphology.

In certain example embodiments, the method further comprises correlating the directly captured material, indirectly captured material, or both to a position in the sample on the fixed addressable array.

In certain example embodiments, comprises assigning pixel coordinates to the image of the sample, image of the copied sample polynucleotides, image of the copied barcoded recognition molecules, or a combination thereof and coordinating the assigned pixel coordinates to the x,y position in the fixed addressable array.

In certain example embodiments, the method further comprises assigning a cell type, cell state, or both to cells in the sample.

In certain example embodiments, the method further comprises staining the sample and optionally recording the morphology of the stained sample.

In certain example embodiments, the method further comprises permeabilizing the sample.

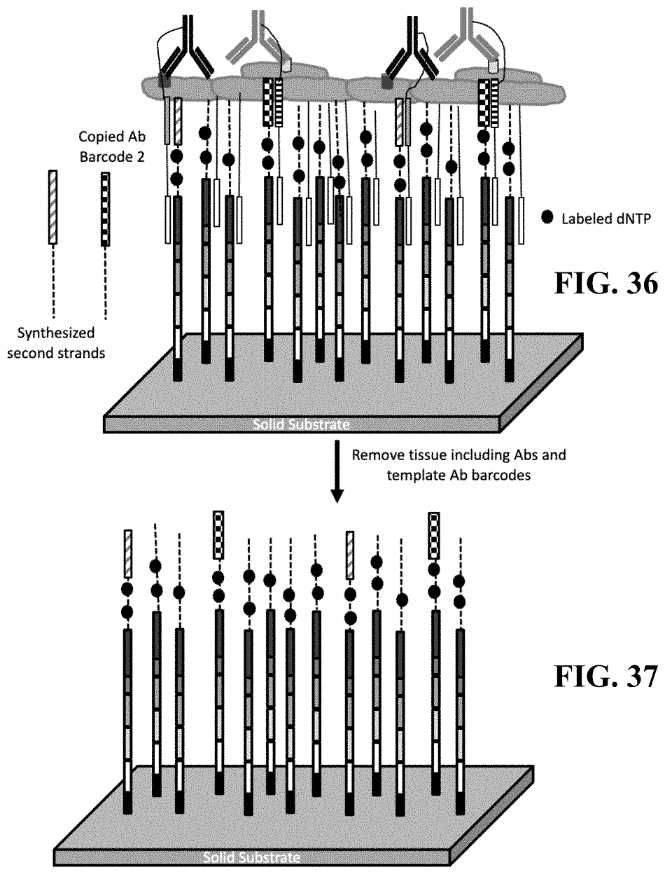

In certain example embodiments, copying the captured sample polynucleotide(s), the captured barcoded recognition molecule barcode(s), or both, comprises incorporating labeled dNTP's into the copied sample polynucleotide(s), a copied barcoded recognition molecule barcode(s), or both (see e.g., FIGS. 36-37, 42, and 55).

In certain example embodiments, detecting the copied sample polynucleotides, copied barcoded recognition molecule barcodes, or both comprises detecting the labeled dNTPs incorporated into the copied sample polynucleotide(s), a copied barcoded recognition molecule barcode(s), or both. (see e.g., FIGS. 36-37, 42, and 55-57).

In certain example embodiments, detecting the labeled dNTPs incorporated into the copied sample polynucleotide(s), a copied barcoded recognition molecule barcode(s), or both comprises imaging the labeled dNTPs.

In certain example embodiments, copying the captured sample polynucleotide comprises synthesizing a complementary strand from the array probe using the captured sample polynucleotide as a template (see e.g., FIG. 42).

In certain example embodiments, copying the barcoded recognition molecule barcode comprises synthesizing a complementary strand from the array probe using the captured barcoded recognition molecule barcode as a template (see e.g., FIG. 36).

In certain example embodiments, the method further comprises specifically binding a concatemer to a copied barcode recognition molecule(s), copied sample polynucleotide(s), or both.

In certain example embodiments, the sample, the barcoded recognition molecule, captured barcoded recognition molecule barcode, captured sample polynucleotide or a combination thereof is/are removed prior to detecting the copied barcoded recognition molecule barcode, copied polynucleotide, or both (see e.g., FIGS. 31-35, 37).

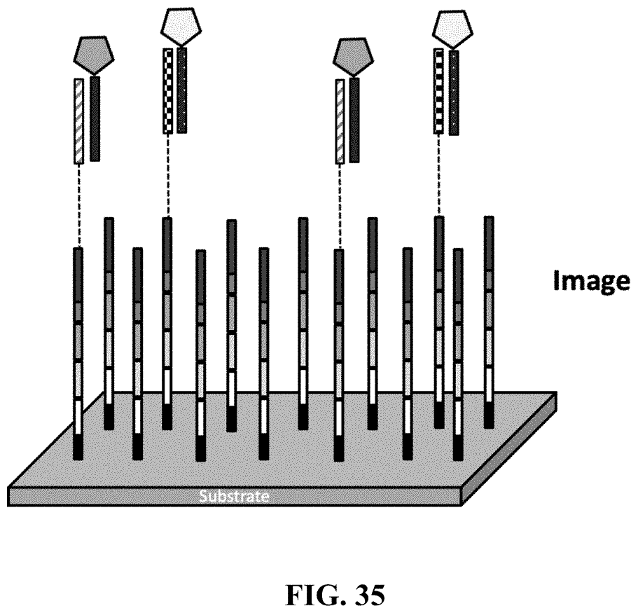

In certain example embodiments, detecting the copied barcoded recognition molecule barcode, the copied sample polynucleotide, or both comprises specifically binding one or more detectable probes to the copied barcoded recognition molecule barcode, the copied sample polynucleotide, concatemer, or a combination thereof (see e.g., FIGS. 34-35 and 50).

In certain example embodiments, detecting the copied barcoded recognition molecule barcode comprises specifically binding a first detectable probe to a first copied barcoded recognition molecule barcode corresponding to a first target and optionally specifically binding a second detectable probe to a second copied barcoded recognition molecule barcode corresponding to a second target. (see e.g., FIG. 35)

In certain example embodiments, detecting the copied sample polynucleotide comprises specifically binding a first detectable probe to a first copied sample polynucleotide corresponding to a first sample polynucleotide and optionally specifically binding a second detectable probe to a second copied sample polynucleotide corresponding to a second sample polynucleotide.

In certain example embodiments, the first detectable probe specifically bound to the first copied barcoded recognition molecule barcode is removed prior to specifically binding the second detectable probe to the second copied barcoded recognition molecule barcode.

In certain example embodiments, specifically binding a first detectable probe to a first copied barcoded recognition molecule barcode and specifically binding a second detectable probe to a second copied barcoded recognition molecule barcode occurs simultaneously.

In certain example embodiments, the first detectable probe specifically bound to the first copied sample polynucleotide is removed prior to specifically binding the second detectable probe to the second copied sample polynucleotide.

In certain example embodiments, specifically binding a first detectable probe to a first copied sample polynucleotide and specifically binding a second detectable probe to a second copied sample polynucleotide occurs simultaneously.

In certain example embodiments, the first detectable probe comprises a first label and the second detectable probe comprises a second label and wherein the first label and the second label are different.

In certain example embodiments, the first detectable probe comprises a first label and the second detectable probe comprises a second label and wherein the first label and the second label are the same.

In certain example embodiments, the detectable label on the optionally present non-barcoded recognition molecule is different than a first label or a second label present on the first or second detectable probes when present.

In certain example embodiments, the method further comprises preparing a cDNA library from the captured sample polynucleotide.

In certain example embodiments, preparing a cDNA library comprises preparing a cDNA library PCR product, or both from the copied sample polynucleotide.

In certain example embodiments, the method further comprises preparing a cDNA library, PCR product, or both from the copied barcoded recognition molecule barcode.

In certain example embodiments, the copied sample polynucleotides and array probes are released from the fixed addressable array prior to generating a cDNA library, PCR product, or both.

In certain example embodiments, the copied barcoded recognition molecule barcodes and array probes are released from the fixed addressable array prior to generating a cDNA library, PCR product, or both.

In certain example embodiments, the method further comprises sequencing the cDNA library, PCR product, or both.

In certain example embodiments, the method further comprises correlating each of the cDNA molecules in the cDNA library, each PCR product, or both to a position in the sample on the fixed addressable array.

In certain example embodiments, the method further comprises assigning a cell type, cell subtype, cell state, or any combination thereof to the plurality of cells in the sample, the assigning comprising detecting differential expression of the cDNA molecules, PCR product(s), or both, to generate a gene and/or protein signature and identifying cell type, cell subtype, cell state, or any combination thereof based on the gene signature at positions in the sample.

In certain example embodiments, the barcoded recognition molecule, the non-barcoded recognition molecule, or both comprise a polynucleotide guided nucleic acid targeting system or molecule thereof, an antibody or fragment thereof, an aptamer, or a combination thereof.

In certain example embodiments, the polynucleotide guided nucleic acid targeting system or molecule thereof is a CRISPR-Cas system or a combination thereof.

In certain example embodiments, the method further comprises sequencing the operatively coupled material.

In certain example embodiments, the method further comprises sequencing the directly captured material, indirectly captured material, or both.

In certain example embodiments, the fixed addressable array further comprises a substrate, wherein the plurality of array probes of the fixed addressable array are coupled to the substrate.

In certain example embodiments, the substrate comprises a solid substrate, a semi-solid substrate, a liquid substrate, or a hydrogel.

In certain example embodiments, the substrate comprises a polymer, wherein the polymer optionally forms a layer on a surface of the substrate, and wherein the plurality of array probes are coupled to the polymer.

In certain example embodiments, the substrate comprises a plurality of wells, wherein the plurality of wells is optionally organized in an array.

In certain example embodiments, the substrate comprises an optically transparent material.

In certain example embodiments, the method further comprises releasing the plurality of array probes from the substrate.

In certain example embodiments, releasing comprises cleaving a cleavable linker on each array probe of the plurality of array probes.

In certain example embodiments, the cleavable linker is a restriction enzyme site, and releasing the plurality of array probes comprises cleaving the restriction enzyme site with a restriction enzyme specific to the restriction site.

In certain example embodiments, the method further comprises depositing one or more CRISPR-Cas systems or components thereof onto the substrate.

In certain example embodiments, the one or more CRISPR-Cas systems or components thereof are deposited at each x,y position defined by the fixed addressable array.

In certain example embodiments, each of the one or more CRISPR-Cas systems or components thereof comprises a guide sequence, wherein each guide sequence is coupled an array probe in the plurality of array probes.

In certain example embodiments, the guide sequence is coupled to the spatial barcode of the array probe.

In certain example embodiments, the method further comprises delivering one or more CRISPR-Cas systems or components thereof to the sample prior to depositing the sample on the fixed addressable array.

In certain example embodiments, the fixed addressable array is contained in a droplet.

In certain example embodiments, the sample is a tissue sample.

In certain example embodiments, detecting the copied sample polynucleotides, the labeled dNTPs, the copied barcoded recognition molecule barcodes, the sample, or any combination thereof comprises in-situ sequencing, laser scanning, fluorescent microscopy, DNA microscopy, FISH, smFISH, in situ PCR, or any combination thereof.

In certain example embodiments, the method further comprises detecting the labeled recognition molecule.

In certain example embodiments, the labeled recognition molecule comprises a polynucleotide guided nucleic acid targeting system or molecule thereof, an antibody or fragment thereof, an aptamer, or a combination thereof.

In certain example embodiments, the polynucleotide guided nucleic acid targeting system or molecule thereof is a CRISPR-Cas system or a combination thereof.

In certain example embodiments, detecting comprises imaging the labeled recognition molecule.

In certain example embodiments, operatively coupling the material from the sample to the bead array comprises depositing the sample comprising a plurality of cells on the decoded array and allowing at least some of the plurality of conductive beads to each couple to one or more of the plurality of cells.

In certain example embodiments, each conductive bead is a magnetic bead.

In certain example embodiments, the spatial barcode is color coded or quenched.

In certain example embodiments, the target molecule of at least one of the bead probes is captured by a capture probe of an array probe of the fixed addressable array.

In certain example embodiments, the decoded bead array is decoded by sequential hybridization and detection of the color coded or quenched spatial barcodes. Optical bead array decoding can be performed by any suitable method. Methods of optically decoding bead arrays based have been described in e.g., Yuan et al., Sci. Rep. 2014. Oct. 24(4):6755; Linz et al., Lab Chip, 2017. 17(6):10776-1082; Yuan et al. 2018. BioRxiv. doi: https://doi.org/10.1101/355677; Kermani et al., Sensors and Actuators B: Chemical. 2006. 117(1): 282-285, which are incorporated by reference herein and can be adapted for use with the present invention.

In certain example embodiments, the method further comprises calculating for background, calculating for errors, or both using the quenched spatial barcodes.

In certain example embodiments, decoding the bead array further comprises in-situ sequencing, laser scanning, DNA microscopy, fluorescent microscopy, laser scanning, FISH, smFISH, in-situ PCR, or a combination thereof.

In certain example embodiments, in-situ sequence comprises Illumina sequencing or Nanopore sequencing.

In certain example embodiments, one or more array probes comprises an oligonucleotide sequence, wherein one or more bead probes comprises an oligonucleotide sequence, or both.

In certain example embodiments, the capture molecule(s), the target molecule(s), or both comprises a Tn5 sequence, a 16S sequence, a poly(d)T sequence, a poly(d)A, sequence a random hexamer sequence, a trypsin molecule, an antibody, an aptamer, a Protein Epitope Signature Tag (PrEST) sequence, a DNA sequence or structural variation, or a combination thereof.

In certain example embodiments, the DNA sequence or structural variation is a single nucleotide polymorphism or a copy number variation.

In certain example embodiments, one or more array probes, one or more bead probes, or both further comprise one or more of a unique molecular identifier (UMI), an adapter sequence, and a primer sequence.

In certain example embodiments, the method further comprises ablating a single layer of the sample and performing the step of operatively coupling material from the sample in a second layer of the sample.

In certain example embodiments, any one or more of the steps is automated.

### Depositing Spatial Barcodes

The dispensing or depositing of spatial barcodes on a solid substrate can be performed in a variety of ways, depending on the type of spatial barcode, type of solid substrate, and further processing of capture material. The spatial barcodes are deposited in individual discrete volumes, which may include spots on the solid substrate, droplets, or other defined area.

An “individual discrete volume” is a discrete volume or discrete space, such as a container, receptacle, or other defined volume or space that can be defined by properties that prevent and/or inhibit migration of nucleic acids and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the used of non-walled, or semipermeable is that some reagents, such as buffers, chemical activators, or other agents maybe passed in through the discrete volume, while other material, such as target molecules, maybe maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are the wells of a microplate. In certain example embodiments, the microplate is a 96 well, a 384 well, or a 1536 well microplate.

Deposition of barcodes can include use of inkjet technologies or contact printing. Inkjet printing technology deposits small droplets of liquid onto the solid substrate, typically using piezoelectric, thermal acoustic, or continuous flow technologies. (Hughes et al, 2001). Contact printing can also be utilized, relying physical deposition of small volume of liquid from a variety of pin tools, including solid or split pins, onto the solid substrate. In particular embodiments, the spatial barcodes are provided in droplets, as discussed elsewhere herein, and deposition can include use of inkjet or fluorescence activated cell sorting (FACS) technologies. Once assembled the droplet, in some embodiments, can be reversed, and the water phase comprising multiple copies of the same oligonucleotide attached to the surface via covalent or non-covalent binding enables control of size of the spots on the solid substrate. Depositing can be performed randomly or in an ordered fashion. In particular embodiments, depositing the spatial barcode comprises the binding of the spatial barcode to the solid substrate, and may be performed by building the spatial barcode on the solid surface utilizing deposition technologies. Preferred sizes of deposition are less than about 5 μm, 4 μm, 3 μm, 2 μm, 1 μm, 900 nm, 800 nm, 700 nm, 600 nm, 500 nm, 400 nm, 300 nm, 200 nm or about 100 nm, 50 nm, or less. The smaller the spots on the solid substrate allows gathering of a more complete map of the sample.

### Spatial Barcode

Methods and systems disclosed herein utilize a plurality of spatial barcodes. Each spatial barcode acts as a two-dimensional coordinate identifier, providing x,y coordinates for a location on a solid substrate. In certain example embodiments, the spatial barcode is provided in a droplet. The spatial barcode, in some embodiments, may be included on a bead. The spatial barcode can comprise an oligonucleotide, which, in some embodiments, is appended or associated with a bead. In particular embodiments, a plurality of spatial barcodes is linked or appended on the bead or in a droplet.

### Oligonucleotide Barcode

An oligonucleotide spatial barcode can be a short sequence of nucleotides (for example, DNA or RNA) that is used as an identifier for the location on the solid substrate of an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a position on the solid substrate corresponding to a capture material from the sample, such as a protein, or cDNA such that multiple species can be sequenced together.

Barcoding may be performed based on any of the compositions or methods disclosed in patent publication WO 2014047561 A1, Compositions and methods for labeling of agents, incorporated herein in its entirety *(See Example 1-8 for discussion of multiple approaches). Additional approaches for barcode synthesis disclosed in PCT/US2018057173 are incorporated herein by reference, in particular [0145]-[0195]. In certain embodiments, barcoding uses an error correcting scheme (T. K. Moon, Error Correction Coding: Mathematical Methods and Algorithms (Wiley, New York, ed. 1, 2005)). In certain example embodiments, capture molecules can be resolved based on the barcode associated with each spatial location that can be correlated to a location within the sample on the solid substrate. In particular embodiments, the method comprises building the spatial barcode on the solid substrate, in some instances building the spatial barcode comprises bridge PCR or solid extension.

In some embodiments, building the spatial barcode on the surface includes the use of padlock probes. A method of building the spatial barcode on the surface comprises distributing oligonucleotide sequences on the solid substrate, adding padlock probes, and amplifying and decoding the oligonucleotides on the surface. In embodiments, DNA oligonucleotides can be randomly distributed on the solid substrate, for example a polymer surface with available —COOH and/or —OH groups. Preferred embodiments distribute the oligonucleotides to allow about 1 μm of space between each of the distributed oligonucleotides when coupled. Padlock probes are added to the simultaneously amplify and decode the DNA oligonucleotides on the surface into rolling circle amplified products. In particular embodiments, the rolling circle amplified products are about 0.5 to about 1 μm.

Another embodiment of building oligonucleotide sequence on the solid substrate includes building and decoding the probe using one reaction with DNA microscopy. Spatial encoding is controlled by diffusion speed, and advantageously allows the steps of probe building and decoding to occur together in one reaction.

Another embodiment allows the use of Affymetrix arrays that can be transferred to a gel as a solid substrate using the 5′ ends of the array, the gel array can then be used as the solid substrate comprising the spatial barcodes. Capture molecules can then be added to the oligonucleotide sequences.

In preferred embodiments, sequencing is performed using unique molecular identifiers (UMI). The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR or multiple displacement amplification (MDA). A UMI may be unique for each spatial barcode.

In certain embodiments, an UMI with a random sequence of between 4 and 20 base pairs is added to a template, which is amplified and sequenced. In preferred embodiments, the UMI is added to the 5′ end of the template. Sequencing allows for high resolution reads, enabling accurate detection of true variants. As used herein, a “true variant” will be present in every amplified product originating from the original clone as identified by aligning all products with a UMI. Each clone amplified will have a different random UMI that will indicate that the amplified product originated from that clone. Background caused by the fidelity of the amplification process can be eliminated because true variants will be present in all amplified products and background representing random error will only be present in single amplification products (See e.g., Islam S. et al., 2014. Nature Methods No:11, 163-166). Not being bound by a theory, the UMI's are designed such that assignment to the original can take place despite up to 4-7 errors during amplification or sequencing. Not being bound by a theory, an UMI may be used to discriminate between true barcode sequences.

Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of spatial nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example spatial barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associate with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an spatial barcode may be a nucleic acid including a barcode and a hybridization site for a specific primer. In particular embodiments, a set of spatial barcodes includes a unique primer specific barcode made, for example, using a randomized oligo type NNNNNNNNNNNN.

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated spatial nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, a spatial barcode further comprises a sequencing adaptor. In some embodiments, a spatial barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, a spatial barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing-based methods. For example, variable length probes or primers can be used to distinguish barcodes (for example, spatial barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an spatial barcode. The resultant nucleic acid barcode concatemer can be pooled with other such concatemers and sequenced. The sequencing reads can be used to identify which target molecules were originally present in which discrete volumes.

### Barcodes Reversibly Coupled to Solid Substrate

In some embodiments, the spatial barcodes can be reversibly coupled to a solid or semisolid substrate. In some embodiments, the spatial barcodes further comprise a nucleic acid capture sequence that specifically binds to the target nucleic acids and/or a specific binding agent that specifically binds to the target molecules. In specific embodiments, the spatial barcodes include two or more populations of spatial barcodes, wherein a first population comprises the nucleic acid capture sequence and a second population comprises the specific binding agent that specifically binds to the target molecules. In some examples, the first population of spatial barcodes further comprises a target nucleic acid barcode, wherein the target nucleic acid barcode identifies the population as one that labels nucleic acids. In some examples, the second population of spatial barcodes further comprises a target molecule barcode, wherein the target molecule barcode identifies the population as one that labels target molecules.

Barcode with Cleavage Sites

A nucleic acid barcode may be cleavable from a specific binding agent, for example, after the specific binding agent has bound to a target molecule. In some embodiments, the spatial barcode further comprises one or more cleavage sites. Linkers can be as described, for example, in PCT/US18/57173 at [0093]-[0102]. In embodiments, the linker is thermally, chemically or enzymatically cleavable linker. In some examples, at least one cleavage site is oriented such that cleavage at that site releases the spatial barcode from a substrate, such as a bead, for example a hydrogel bead, to which it is coupled. In some examples, at least one cleavage site is oriented such that the cleavage at the site releases the spatial barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage. In a particular embodiment, the cleavable linker comprises a d(U) linker.

### Barcode Adapters

In some embodiments, the target molecule is attached to a spatial barcode receiving adapter, such as a nucleic acid. In some examples, the spatial barcode receiving adapter comprises an overhang and the spatial barcode comprises a sequence capable of hybridizing to the overhang. A barcode receiving adapter is a molecule configured to accept or receive a nucleic acid barcode, such as a spatial nucleic acid barcode. For example, a barcode receiving adapter can include a single-stranded nucleic acid sequence (for example, an overhang) capable of hybridizing to a given barcode (for example, a spatial barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which a spatial barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

### Sequencing Adapters

As used herein, sequence adapters or sequencing adapters or adapters include primers that may include additional sequences involved in for example, but not limited to, flowcell binding, cluster generation, library generation, sequencing primers, sequences for Seq-Well, and/or custom read sequencing primers. In certain embodiments, the sequencing adapters are tailored to the end0use, for example, when a flowcell or other non-bead-based technology is used, additional sequencing adapters can be utilized for library generation.

### Universal Primer Recognition Sequences

The present invention may encompass incorporation of SMART sequences into the library. Switching mechanism at 5′ end of RNA template (SMART) is a technology that allows the efficient incorporation of known sequences at both ends of cDNA during first strand synthesis, without adaptor ligation. The presence of these known sequences is crucial for a number of downstream applications including amplification, RACE, and library construction. While a wide variety of technologies can be employed to take advantage of these known sequences, the simplicity and efficiency of the single-step SMART process permits unparalleled sensitivity and ensures that full-length cDNA is generated and amplified. See, e.g., Zhu et al., 2001, Biotechniques. 30 (4): 892-7.

A pooled set of nucleic acids that are tagged refer to a plurality of nucleic acid molecules that results from incorporating an identifiable sequence tag into a pool of sample-tagged nucleic acids, by any of various methods. In some embodiments, the tag serves instead as a minimal sequence adapter for adding nucleic acids onto sample-tagged nucleic acids, rendering the pool compatible with a particular DNA sequencing platform or amplification strategy.

The barcodes herein may comprise one or more detectable tags. In some examples, a detectable tag may comprise a detectable oligonucleotide tag that can be detected by sequencing of its nucleotide sequence and/or by detecting non-nucleic acid detectable moieties to which it may be attached.

The oligonucleotide tags may be randomly selected from a diverse plurality of oligonucleotide tags. In some instances, an oligonucleotide tag may be present once in a plurality or it may be present multiple times in a plurality. In the latter instance, the plurality of tags may be comprised of a number of subsets each comprising a plurality of identical tags. In some important embodiments, these subsets are physically separate from each other. Physical separation may be achieved by providing the subsets in separate wells of a multiwell plate or separate droplets from an emulsion. It is the random selection and thus combination of oligonucleotide tags that results in a unique label. Accordingly, the number of distinct (i.e., different) oligonucleotide tags required to uniquely label a plurality of agents can be far less than the number of agents being labeled. This is particularly advantageous when the number of agents is large (e.g., when the agents are members of a library).

The oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the non-nucleic acid detectable moiety.

In some embodiments, a detectable oligonucleotide tag comprises one or more non-oligonucleotide detectable moieties. Examples of detectable moieties include fluorophores, microparticles including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, haptens, and other detectable moieties known to those skilled in the art.

Thus, detectable oligonucleotide tags may be, but are not limited to, oligonucleotides comprising unique nucleotide sequences, oligonucleotides comprising detectable moieties, and oligonucleotides comprising both unique nucleotide sequences and detectable moieties.

In some cases, the detectable tag comprises a labeling substance, which is detectable by spectroscopic, photochemical, biochemical, immunochemical, electrical, optical or chemical means. Such tags include biotin for staining with labeled streptavidin conjugate, magnetic beads (e.g., Dynabeads®), fluorescent dyes (e.g., fluorescein, Texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Detectable tags may be detected by many methods. For example, radiolabels may be detected using photographic film or scintillation counters, and fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

A mixture comprises a plurality of microbeads adorned with combinations of the following elements: bead-specific oligonucleotide barcodes created by the discussed methods; additional oligonucleotide barcode sequences which vary among the oligonucleotides on an individual bead and can therefore be used to differentiate or help identify those individual oligonucleotide molecules; additional oligonucleotide sequences that create substrates for downstream molecular-biological reactions, such as oligo-dT (for reverse transcription of mature mRNAs), specific sequences (for capturing specific portions of the transcriptome, or priming for DNA polymerases and similar enzymes), or random sequences (for priming throughout the transcriptome or genome). In an embodiment, the individual oligonucleotide molecules on the surface of any individual microbead contain all three of these elements, and the third element includes both oligo-dT and a primer sequence.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., ³²P, ¹⁴C, ¹²⁵I, ³H, and ¹³¹I) fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added. Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-l-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-di ethyl amino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. A fluorescent label may be a fluorescent protein, such as blue fluorescent protein, cyan fluorescent protein, green fluorescent protein, red fluorescent protein, yellow fluorescent protein or any photoconvertible protein. Colorimetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code. Advantageously, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation. In some embodiments, the detectable moieties may be quantum dots.

Barcode with Capture Moiety

In some embodiments, a spatial barcode further includes a capture moiety, covalently or non-covalently linked. Thus, in some embodiments the spatial barcode, and anything bound or attached thereto, that include a capture moiety are captured with a specific binding agent that specifically binds the capture moiety. In some embodiments, the capture moiety is adsorbed or otherwise captured on a surface. In specific embodiments, a targeting probe is labeled with biotin, for instance by incorporation of biotin-16-UTP during in vitro transcription, allowing later capture by streptavidin. Other means for labeling, capturing, and detecting a spatial barcode include incorporation of aminoallyl-labeled nucleotides; incorporation of sulfhydryl-labeled nucleotide; incorporation of allyl- or azide-containing nucleotide; and many other methods described in Bioconjugate Techniques (2^{nd} Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments, the specific binding agent has been immobilized for example on a solid support, thereby isolating the spatial barcode.

### Other Barcoding Embodiments

DNA barcoding is also a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102(23):8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

It has been suggested that a desirable locus for DNA barcoding should be standardized so that large databases of sequences for that locus can be developed. Most of the taxa of interest have loci that are sequencable without species-specific PCR primers. CBOL Plant Working Group, “A DNA barcode for land plants” PNAS 106(31):12794-12797 (2009). Further, these putative barcode loci are believed short enough to be easily sequenced with current technology. Kress et al., “DNA barcodes: Genes, genomics, and bioinformatics” PNAS 105(8):2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105(8):2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31):12569 (2009).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94).

Unique Molecular Identifiers are short (usually 4-10 bp) random barcodes added to transcripts during reverse-transcription. They enable sequencing reads to be assigned to individual transcript molecules and thus the removal of amplification noise and biases from RNA-seq data. Since the number of unique barcodes (4N, N—length of UMI) is much smaller than the total number of molecules per cell (˜106), each barcode will typically be assigned to multiple transcripts. Hence, to identify unique molecules both barcode and mapping location (transcript) must be used. UMI-sequencing typically consists of paired-end reads where one read from each pair captures the cell and UMI barcodes while the other read consists of exonic sequence from the transcript. UMI-sequencing typically consists of paired-end reads where one read from each pair captures the cell and UMI barcodes while the other read consists of exonic sequence from the transcript.

In some embodiments, the nucleic acids of the library are flanked by switching mechanism at 5′ end of RNA templates (SMART). SMART is a technology that allows the efficient incorporation of known sequences at both ends of cDNA during first strand synthesis, without adaptor ligation. The presence of these known sequences is crucial for a number of downstream applications including amplification, RACE, and library construction. While a wide variety of technologies can be employed to take advantage of these known sequences, the simplicity and efficiency of the single-step SMART process permits unparalleled sensitivity and ensures that full-length cDNA is generated and amplified. See, e.g., Zhu et al., 2001, Biotechniques. 30 (4): 892-7.

After processing the reads from a UMI experiment, the following conventions are often used: 1. The UMI is added to the read name of the other paired read. 2. Reads are sorted into separate files by barcode. For extremely large, shallow datasets, a barcode may be added to the read name as well to reduce the number of files. A barcode indicates the cell from which mRNA is captured (e.g., Drop-Seq or Seq-Well).

### Split-Pool Barcoding

In some embodiments, the nucleic acids molecules, e.g., the fragmented genomic DNA and the cDNA, may be barcoded by a split-pool method. In some embodiments, the split-pool method may be performed on a sample comprising nuclei containing the fragmented genomic DNA and the cDNA herein. In such cases, the fragmented genomic DNA and the cDNA remain in nuclei after generation. The nuclei may remain intact during the split-pool process. In certain examples, the nuclei are isolated from cells. For example, the cells may be lysed, and the nuclei are released, but remain intact and contain the fragmented genomic DNA and the cDNA. In certain examples, the nuclei remain in the cells, which are made permeable so the nucleic acids in the cells (e.g., in the nuclei) can access reaction reagents and the fragmented DNA and the cDNA can be generated inside cells.

In general, the split-pool method may comprise: splitting a sample comprising nuclei into discrete volumes in partitions, each partition containing a unique first barcode; ligating the first barcode to nucleic acids in each partition; pooling the discrete partitions to a first pooled sample. The process may be repeated. For example, the split-pool method may further comprise splitting the first pooled sample into discrete partitions, each partition containing a unique second barcode; ligating the second barcode to nucleic acids in each partition; and pooling the discrete partitions to make a second pooled sample. The splitting and pooling steps may be repeated for at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, or at least 500 times.

After split-pool steps, each nucleic acid molecule may comprise one or a combination of barcodes. Since when split, nucleic acid molecules in a nuclei or cell are split together, nucleic acid molecules from or derived from the same cell may receive the same barcode or barcode combination. Such barcode or barcode combination may comprise a unique barcode sequence, which may be used as an identifier of cell origin of the nucleic acid molecules.

In some embodiments, nucleic acids in the split-pool process may comprise ligation handles. The ligation handle may comprise a restriction site for producing an overhang complementary with a first index sequence overhang, and wherein the method further comprises digestion with a restriction enzyme. The ligation handle may comprise a nucleotide sequence complementary with a ligation primer sequence and wherein the overhang complementary with a first index sequence overhang is produced by hybridization of the ligation primer to the ligation handle. The ligation handles may be generated before the split-pool process. For example, the ligation handles may be generated during the fragmentation, tagmentation, and/or RT-PCR process. Alternatively or additionally, the ligation handles may be generated during the split-pool process.

In particular embodiments, the spatial barcodes comprise beads. The beads may be made of any substance, exemplary beads include conductivity coded beads, color-coded beads, or beads to which the spatial barcode is appended. In some embodiments, the bead by virtue of its characteristics, such as unique combination of colors or conductivity properties, is the spatial barcode. In other instances, the spatial barcode is an oligonucleotide appended or attached to the bead.

In particular embodiments, the spatial barcode is chemically linked to the bead. In some preferred embodiments, a plurality of spatial barcodes are attached to the bead. In some embodiments, the spatial barcodes are linked to a spacer that is permanently or reversibly attached to the bead. In particular embodiments, a cleavable linker can be used between the spacer and the bead, between the barcode and a spacer, and/or at junctions of the spatial barcode and additional moieties appended thereto. In an embodiment, the cleavable linkage can be utilized to allow for the release of the molecules. As described in Example 3, cleaving of the spatially position barcodes can allow release into the tissue, with addition of polymer, betaine, and/or MgCl₂ to increase sensitivity. Parallel capture of mRNA molecules on to the released barcode primers otherwise present on the array surface. Accordingly, a restriction site close to the 5′ end of the capture probes comprising the spatial barcode is preferred.

The beads may be comprised of a polymer. Examples of suitable polymers include a hydroxylated methacrylic polymer, a hydroxylated poly(methyl methacrylate), a polystyrene polymer, a polypropylene polymer, a polyethylene polymer agarose, or cellulose. The beads may be functionalized to permit covalent attachment of the agent and/or label. Such functionalization on the support may comprise reactive groups that permit covalent attachment to a label, spatial barcode or other moiety.

In some embodiments, commercially available beads may be utilized, as described herein. Commercial beads by 10×, Becton Dickinson, Illumina, 454, or other prepared beads can be deposited in a random fashion with each bead containing multiple copies of a spatial barcode oligonucleotide sequence. The barcoded oligonucleotide beads can be constructed such that each bead has a unique spatial barcode sequence, but the bead comprises the multiple copies of oligonucleotides all contain an identical spatial barcode sequence.

### Solid Substrate

In some embodiments, the spatial barcodes are deposited on a solid substrate. The solid substrate can be any suitable material or combination of materials. The solid substrate can comprise a gel, a polymer, an imaging fiber, glass, a conductive surface, or any combination thereof. As used herein, “glass” refers to any type of glass including, but not limited to silicate glasses (e.g., soda-lime glass, borosilicate glass, lead glass, aluminosilicate glass, glass-ceramics, and fiber glass), silica-free glasses (e.g., amorphous metals and polymers), and molecular liquids and molten salts. Glasses can contain additives that can modify e.g., the optical properties (e.g., transparency, color, refractivity etc.), conductive properties or other properties of the glass. As used herein, “polymer” refers to a chemical compound formed from a plurality of repeating structural units referred to as monomers “Polymers” are understood to include, but are not limited to, homopolymers, copolymers, such as for example, block, graft, random and alternating copolymers, terpolymers, etc. and blends and modifications thereof. Polymers can be formed by a polymerization reaction in which the plurality of structural units become covalently bonded together. When the monomer units forming the polymer all have the same chemical structure, the polymer is a homopolymer. When the polymer includes two or more monomer units having different chemical structures, the polymer is a copolymer. Exemplary polymers suitable for the solid substrate include, without limitation, polystyrene and its derivatives and co-polymers and PDMS (polydimethylsiloxane) and its derivatives and co-polymers etc. In some embodiments, the solid substrate have a functionalized surface that promotes cell growth, viability, attachment, or any combination thereof. Such functionalization is generally known in the art.

In some embodiments, the solid substrate includes one or more etchings, indentations, markings, or other indicators of direction, spatial barcode array orientation, well or other discrete location orientation or other identifying information or indicators regarding the solid substrate, arrays contained therein, etc. It will be appreciated that such information can be generic such as A, B, C, D, 1, 2, 3, and can be used in x,y coordinate fashion to specifically identify individual discrete locations or areas that can be in or on the solid substrate.

In one preferred embodiment, the solid substrate is a glass slide. The solid substrate can in some instances can be used for cell and tissue culturing while simultaneously allowing for analysis and evaluation of the methods disclosed herein.

A number of substrates and configurations may be used. The devices may be capable of defining multiple individual discrete volumes within the device. As used herein an “individual discrete volume” refers to a discrete space, such as a container, receptacle, or other defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof that can contain a sample within a defined space. Individual discrete volumes may be identified by molecular tags, such as the spatial barcodes as described herein. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain embodiments, the compartment is an aqueous droplet in a water-in-oil emulsion or an oil in water emulsion. In specific embodiments, any of the applications, methods, or systems described herein requiring exact or uniform volumes may employ the use of an acoustic liquid dispenser.

In certain example embodiments, the device comprises a flexible material substrate on which a number of spots may be defined and can comprise a gel. Within each defined spot, reagents of the system described herein are applied to the individual spots. Each spot may contain the same reagents except for a different capture molecule, or guide RNA or set of guide RNAs in instances where CRISPR or other nucleotide editing systems are utilized, or where applicable, a different detection aptamer to screen for multiple targets at once. The guide molecule may be linked to the spatial barcodes described herein. Thus, the systems and devices herein may be able to screen multiple regions of a sample such as a tissue sample, for the presence of the same target, or a limited number of targets, or for the presence of multiple different targets in the sample.

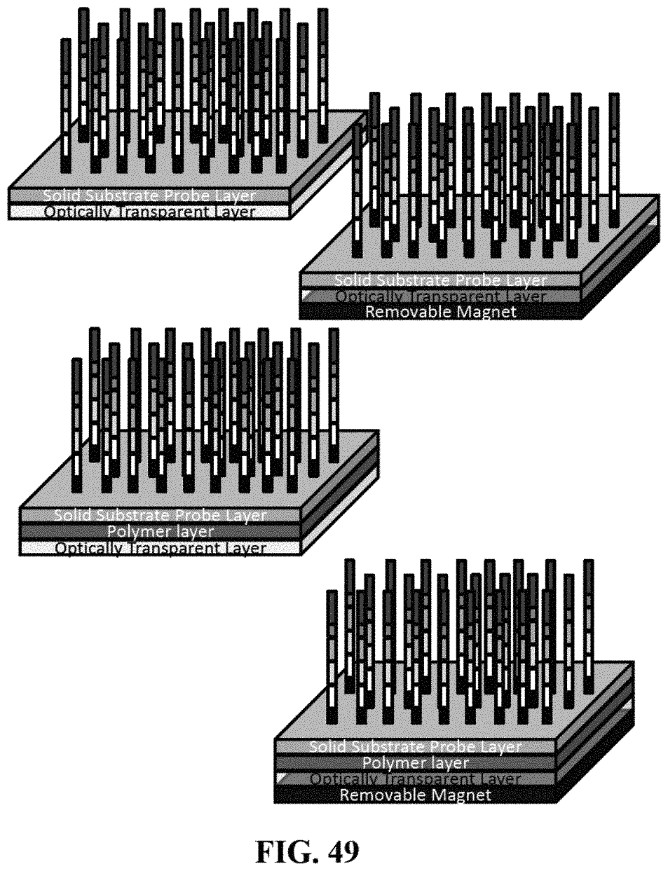

In some embodiments, the solid substrate can be one as shown in FIG. 49. In some embodiments, the solid substrate can be reversibly coupled with a magnet. In some embodiments, coupling of the magnet to the substrate can reversibly hold magnetic beads in place against the spatial barcode array (or fixed array), see e.g., FIGS. 44-48. In some embodiments, the solid substrate can be composed of multiple layers and/or materials.

In some embodiments a single solid substrate can include one or more spatial barcode (or fixed arrays) allowing multiple analyses to be performed in parallel on a single substrate. In some embodiments, a solid substrate have 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more spatial barcode (or fixed arrays).

In some embodiments, the fixed array is an addressable array. As used herein, “array” encompasses any two or three dimensional ordered arrangement of features, where each feature has a unique position in two- or three-dimensional space. Thus, it will be appreciated that each feature in an array can be identified by a unique x,y (two-dimensional arrays) or unique x, y, z coordinate (three-dimensional arrays). Each feature of the array can be any physical, chemical, or biological, composition, property, or aspect that can or has the potential to bind with, react with, contain, fixate, incorporate, or otherwise hold in position a sample or a component thereof. As used herein, “addressable array” refers to an array where the unique position of each feature is predetermined and/or is organized such that each feature and/or its position is otherwise identifiable from each other feature and/or position thereof in the addressable array. Such predetermined and/or organized addressing of the features in an addressable array can allow for detection, measuring, determination, and/or identification of e.g., a specific target present in a sample, a specific sample characteristic(s), and/or response(s) present in a sample, a specific condition or set of conditions applied at each feature that elicits or causes a response in a sample, or any combination thereof, thus providing useable information about the sample or one or more component thereof and/or condition(s) applied to a sample.

Features can be arranged within an array (including an addressable array) such that there is substantially no distance between two or more features, that there is a distance between two or more features, or a combination thereof. In some embodiments, the distance between each feature is the same between each feature of the array. In some embodiments, the distance between each feature of the array can be varied. In some embodiments, the features can be contained in, attached to, integrated with, or otherwise coupled to a substrate (e.g., the solid substrate) or a surface thereof. The number of features can range from 1 to 1,000,000 or more.

In some embodiments, the features are spatial barcodes. In some embodiments, the solid substrate contains an array (such as an addressable array) of spatial barcode arrays (such as a fixed spatial barcode array). In some of these embodiments, each spatial barcode array has the same arrangement of spatial barcodes. In some of these embodiments, at two or more spatial barcode arrays have a different arrangement of spatial barcodes. In some of these embodiments, each spatial barcode array has the same types of spatial barcodes. In some of these embodiments, at two or more spatial barcode arrays have a different collection of spatial barcodes.

In some embodiments, one or more of the features can contain one or more sub features. The sub features can be contained in, attached to, integrated with, or otherwise coupled to the feature and/or substrate (e.g., the solid substrate) or a surface thereof. As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects. In some embodiments, the features can be adsorbed, physisorbed, or chemisorbed to a substrate (e.g., the solid substrate). In some embodiments, the substrate (e.g., the solid substrate) can fix or hold the feature in a specific position within the array. In some embodiments, the features can be formed from voids present in the substrate (e.g., wells or etchings). In some embodiments, the sub features can be adsorbed, physisorbed, or chemisorbed to a substrate (e.g., the solid substrate). In some embodiments, the substrate can fix or hold the sub feature in a specific position within the feature of the array. In some embodiments, the sub features can be formed from voids present in a feature (e.g., void, engraving or etching). Sub features can be arranged within a feature of the array such that there is substantially no distance between two or more sub features, that there is a distance between two or more sub features, or a combination thereof. In some embodiments, the distance between each sub feature is the same between each sub feature. In some embodiments, the distance between each sub feature of the array can be varied. In some embodiments, the sub features can be contained in, attached to, integrated with, or otherwise coupled to a feature, the substrate (e.g., the solid substrate) and/or a surface thereof.

In some embodiments, one or more dimensions (e.g., a length, a width, a height, a diameter, and the like) of the substrate (e.g., the solid substrate) or a feature thereof can range from about 1-1,000 pm, nm, μm, cm, or mm. In some embodiments, one or more dimensions of the substrate (e.g., the solid substrate) can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the largest dimension of the substrate (e.g., the solid substrate) can range from 1-1,000 pm, nm, μm, cm, or mm. In some embodiments, the largest dimension of the substrate (e.g., the solid substrate) can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the smallest dimension of the substrate (e.g., the solid substrate) can range from 1-1,000 pm, nm, μm, cm, or mm. In some embodiments, the smallest dimension of the substrate (e.g., the solid substrate) can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments, the substrate (e.g., the solid substrate) or feature thereof can have a volume. The volume of the substrate (e.g., the solid substrate) or feature thereof can range from about 1-1,000 pm³, nm³, μm³, cm³, mm³, or L³. In some embodiments, the substrate (e.g., the solid substrate) or feature thereof volume can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments, the features are attached or otherwise coupled on one or more surfaces of the substrate (e.g., the solid substrate). As used herein, “surface,” in the context herein, refers to a boundary of an object, such as the substrate (e.g., the solid substrate). The surface can be an interior surface (e.g., the interior boundary of a hollow object), or an exterior or outer boundary of a substrate (e.g., the solid substrate). Generally, the surface of a substrate (e.g., the solid substrate) corresponds to the idealized surface of a three dimensional solid that is topological homeomorphic with the substrate (e.g., the solid substrate). The surface can be an exterior surface or an interior surface. An exterior surface forms the outermost layer of a substrate (e.g., the solid substrate) or device. An interior surface surrounds an inner cavity of a substrate (e.g., the solid substrate) or device, such as the inner cavity of a tube. As an example, both the outside surface of a tube and the inside surface of a tube are part of the surface of the tube. In some embodiments, one or more surfaces can be modified with one or more features. In some embodiments, one or more surfaces can be functionalized to facilitate attachment or coupling of one or more features to the surface.

In some embodiments, one or more dimensions of the surface (e.g., a length, a width, a height, a diameter, and the like) can range from about 1-1,000 pm, nm, μm, cm, or mm. In some embodiments, one or more dimensions of the surface is/are about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the largest dimension of the surface ranges from about 1-1,000 pm, nm, μm, cm, or mm. In some embodiments, the largest dimension of the surface can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the smallest dimension of the surface ranges from about 1-1,000 pm, nm, μ m, cm, or mm. In some embodiments, the smallest dimension of the surface is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments the surface area of the surface ranges from about 1-1,000 pm^{t}, nm², μm², cm², or mm². In some embodiments, the surface area of the surface is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm², nm², μm², cm², or mm².

In some embodiments, the surface has a volume. In some embodiments, the volume of the surface ranges from about 1-1,000 pm³, nm³, μm³, cm³, mm³, or L³. In some embodiments, the surface volume is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments the surface and/or substrate (e.g., the solid substrate) can be porous. In some embodiments the pores of the surface and/or substrate (e.g., the solid substrate) can be substantially homogenous. In some embodiments the pores of the surface and/or substrate can be heterogenous. Pores can have any irregular or regular shape. In some embodiments the surface and/or substrate a population of pores can have an average diameter, average largest dimension, and/or average smallest dimension that can range from 1-1,000 pm, nm, μm, cm, or mm. In some embodiments, the average diameter, average largest dimension, and/or average smallest dimension of the a population of pores is/are about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments, one or more pores of the substrate (e.g., the solid substrate) and/or surface has a diameter, a largest dimension, and/or a smallest dimension that ranges from about 1-1,000 pm, nm, μm, cm, or mm. In some embodiments one or more pores of the substrate and/or surface has a diameter, a largest dimension, and/or a smallest dimension that is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments, the population of pores of the substrate (e.g., the solid substrate) and/or surface has a total pore volume. In some embodiments, the total pore volume of the substrate and/or surface ranges from about 1-1,000 pm³, nm³, μm³, cm³, mm³, or L³. In some embodiments, the total poor volume is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments, all or one or more parts of the substrate and/or surface is/are opaque. In some embodiments, all or one or more parts of the substrate and/or surface is/are transparent. In some embodiments, all or one or more parts of the substrate and/or surface is/are semi-transparent. In some embodiments, all or one or more parts of the substrate and/or surface is/are selectively opaque, selectively semi-transparent, selectively transparent, or any combination thereof. Selectively opaque, selectively transparent, and selectively transparent materials are those that are opaque, semi-transparent, or transparent as to a particular or selected wavelength or range of wavelengths of light. In some embodiments, one or more of the materials of the solid substrate is optically transparent. As used herein, “optically transparent” or “transparent” (used interchangeably herein) refers to the ability of a material to allow about 99, 99.1, 99.2, 99.3, 99.4, 99.5, 99.6, 99.7, 99.8, 99.9, or 100% of light waves of one or more wavelengths pass through. As used herein, “semi-transparent” refers to the ability of material to allow greater than 0 but less than 99 percent of light waves of one or more wavelengths to pass through. As used in this context herein, “opaque” refers to the ability of a material to block substantially all light waves of one or more wavelengths to pass through. Transparent and/or semi-transparent materials can be advantageous for applications where imaging of a sample is employed and/or light needs to reach a sample (such as to induce a reaction in the sample (such as to fix or cleave molecules)). Opaque materials can be advantageous for use in parts between samples or sections to reduce light noise from other samples. Selectively transparent, selectively semi-transparent, and selectively opaque materials are advantageous when only certain wavelengths of light are desired to pass through to or from a sample.

The substrate and/or surface can be completely composed of or include any suitable material(s). Suitable materials include, but are not limited to, glass, ceramics, polymers, gels, hydrogels, adhesives, metals, metalloids, metal alloys, non-metals, crystals, fibrous material, and combinations thereof. The substrate and/or surface can be composed of a biocompatible material.

The term “biocompatible”, as used herein, refers to a substance or object that performs its desired function when introduced into an organism without inducing significant inflammatory response, immunogenicity, or cytotoxicity to native cells, tissues, or organs, or to cells, tissues, or organs introduced with the substance or object. For example, a biocompatible product is a product that performs its desired function when introduced into an organism without inducing significant inflammatory response, immunogenicity, or cytotoxicity to native cells, tissues, or organs.

Biocompatibility, as used herein, can be quantified using the following in vivo biocompatibility assay. A material or product is considered biocompatible if it produces, in a test of biocompatibility related to immune system reaction, less than 50%, 45%, 40%, 35%, 30%, 25%, 20%, 15%, 10%, 8%, 6%, 5%, 4%, 3%, 2%, or 1% of the reaction, in the same test of biocompatibility, produced by a material or product the same as the test material or product except for a lack of the surface modification on the test material or product. Examples of useful biocompatibility tests include measuring and assessing cytotoxicity in cell culture, inflammatory response after implantation (such as by fluorescence detection of cathepsin activity), and immune system cells recruited to implant (for example, macrophages and neutrophils).

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be a three-dimensional network (e.g., the repeat units are linked together left and right, front and back, up and down), a two-dimensional network (e.g., the repeat units are linked together left, right, up, and down in a sheet form), or a one-dimensional network (e.g., the repeat units are linked left and right to form a chain). “Polymers” can be composed, natural monomers or synthetic monomers and combinations thereof. The polymers can be biologic (e.g., the monomers are biologically important (e.g., an amino acid), natural, or synthetic. As used interchangeably herein, “polymer blend” and “polymer mixture” refers to a macroscopically homogenous mixture of two or more different species of polymers. Unlike a copolymer, where the monomeric polymers are covalently linked, the constituents of a “polymer blend” and “polymer mixture” are separable by physical means and does not require covalent bonds to be broken. A “polymer blend” can have two or more (e.g., 2, 3, 4, 5, 6, 7, 8, 9, 10 or more) different polymer constituents.

Exemplary synthetic polymers include, without limitation, poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivatized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

As used herein, “glass” refers to any type of glass including, but not limited to silicate glasses (e.g., soda-lime glass, borosilicate glass, lead glass, aluminosilicate glass, glass-ceramics, and fiber glass), silica-free glasses (e.g., amorphous metals and polymers), and molecular liquids and molten salts. Glasses can contain additives that can modify e.g., the optical properties (e.g., transparency, color, refractivity etc.), conductive properties or other properties of the glass.

As used herein, “metal” refers to Li, Be, Na, Mg, Al, K, Ca, Sc, Ti, V, Cr, Mn, Fe, Co, Ni, Cu, Zn, Ga, Rb, Sr, Y, Zr, Nb, Mo, Tc, Ru, Rh, Pd, Ag, Cd, In, Sn, Cs, Ba, La, Ce, Pr, Nd, Pm, Sm, Eu, Gd, Tb, Dy, Ho, Er, Rm, Yb, Lu, Hf, W, Re, Os, Ir, Pt, Au, Hg, Tl, Pb, Bi, Po, Ra, Ac, Th, Pa, U, Np, Am, Cm, Bk, Cf, Es, Fm, Md, No, Lr, Rf, Db, Sg, Bh, Hs, Mt, Ds, Rg, Cn, Nh, Fl, Mc, Lv, and combinations thereof. As used herein, “metalloid” refers to B, Si, Ge, As, Sb, Te, At, and combinations thereof. As used herein, “non-metal” refers to He, H, C, N, O, F, Ne, P, S, Cl, Ar, Se, Br, Kr, I, Xe, Rn, and combinations thereof.

As used herein, “fibrous material” refers to any bulk material composed of a plurality of fibers. The fibers the fibrous material can be composed of glass, biological polymers (e.g., proteins, polynucleotides), metals, metalloids, non-metals, carbon nanostructures, polymers, crystals, ceramics, metal alloys, and combinations thereof. The fibers can be formed of natural or synthetic materials. The fibrous material can form any usable form, such as a sheet, membrane, strip, tape, slide, fiber, mesh, and the like. The fibrous material can be a flexible, semi-flexible, or inflexible material. Generally, fibrous materials where the individual fibers are loosely coupled to or associated with each other will be more flexible than those where the individual fibers are more tightly coupled or associated with each other. Exemplary fibrous materials include, but are not limited to paper sheets, paper strips and paper tapes, polymeric membranes, fabrics, and fibrous glass membranes.

In some embodiments, all or one or more parts of the surface and/or the substrate can be hydrophilic. In some embodiments, all or one or more parts of the surface and/or the substrate can be hydrophobic. In some embodiments, all or one or more parts of the surface and/or substrate can be superhydrophobic. In some embodiments, patterns on the surface and/or substrate can be formed by specific placement of hydrophobic and/or hydrophilic materials. In some embodiments, such patterns can, without limitation, form features of the array and/or form conduits to provide sample, reactants, features, and the like to one or more regions of the array. As used herein, “hydrophilic”, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the molecule is considered hydrophilic. As used herein, “hydrophobic”, refers to molecules which have a greater affinity for, or solubility in an organic solvent as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the molecule is considered hydrophobic. In some embodiments, hydrophobic and hydrophilic regions can be formed by particular materials that are hydrophobic or hydrophilic or can be formed by changing the texture of a surface (e.g., by etching, scoring, etc.) such that the contact angle or other interaction of water or liquid with the surface is changed such that that region such that it is hydrophobic or hydrophilic.

In some embodiments, the suitable material can be a hydrophobic material. Suitable hydrophobic materials include, but are not limited to: acrylics (e.g., acrylic, acrylonitrile, acrylamide, and maleic anhydride polymers), polyamides and polyimides, carbonates (e.g., Bisphenol A-based carbonates), polydienes, polyesters, polyethers, polyfluorocarbons, polyolefins (e.g., polyethylene, polypropylene, and copolymers thereof), polystyrenes and copolymers thereof, polyvinyl acetals, polyvinyl chlorides and polyvinylidene chlorides, poly vinyl ethers and polyvinyl ketones, polyvinylpyridines and polyvinypyrrolidones, Aculon's Transition Metal Complex coting, SLIPS coating material (Adaptive Surface Technologies), and any combination thereof.

In some embodiments, the suitable material can be composed of or include a superhydrophobic material. Suitable superhydrophobic materials include, but are not limited to manganese oxide polystyrene, zinc oxide polystyrene, precipitated calcium carbonate, carbon nanotubes, silica nano-coatings, fluorinated silanes, and flurophopolymer coatings. See e.g., Meng et al. 2008, The Journal of Physical Chemistry C. 112 (30): 11454-11458; Hu et al. 2009. Colloids and Surfaces A: Physicochemical and Engineering Aspects. 351 (1-3): 65-70; Lin et al., Colloids and Surfaces A: Physicochemical and Engineering Aspects. 421: 51-62; Das et al., RSC Advances. 4 (98): 54989-54997. doi:10.1039/C4RA10171E; Torun et al., 2018. Macromolecules. 51 (23): 10011-10020; Warsinger et al. 2015., Colloids and Surfaces A: Physicochemical and Engineering Aspects. 421: 51-62; Servi et al. 2017., Journal of Membrane Science. Elsevier BV. 523: 470-479

In some embodiments, the suitable material can be composed of or include a hydrophilic material. Hydrophilic materials include, but are not limited to, hydrophilic polymers such as poly(N-vinyl lactams), poly(vinylpyrrolidone), poly(ethylene oxide), poly(propylene oxide), polyacrylamides, cellulosics, methyl cellulose, polyanhydrides, polyacrylic acids, polyvinyl alcohols, polyvinyl ethers, alkylphenol ethoxylates, complex polyol monoesters, polyoxyethylene esters of oleic acid, polyoxyethylene sorbitan esters of oleic acid, and sorbitan esters of fatty acids; inorganic hydrophilic materials such as inorganic oxide, gold, zeolite, and diamond-like carbon; and surfactants such as Triton X-100, Tween, Sodium dodecyl sulfate (SDS), ammonium lauryl sulfate, alkyl sulfate salts, sodium lauryl ether sulfate (SLES), alkyl benzene sulfonate, soaps, fatty acid salts, cetyl trimethylammonium bromide (CTAB) a.k.a. hexadecyl trimethyl ammonium bromide, alkyltrimethylammonium salts, cetylpyridinium chloride (CPC), polyethoxylated tallow amine (POEA), benzalkonium chloride (BAC), benzethonium chloride (BZT), dodecyl betaine, dodecyl dimethylamine oxide, cocamidopropyl betaine, coco ampho glycinate alkyl poly(ethylene oxide), copolymers of poly(ethylene oxide) and poly(propylene oxide) (commercially called Poloxamers or Poloxamines), alkyl polyglucosides, fatty alcohols, cocamide MEA, cocamide DEA, cocamide TEA, Adhesives Research (AR) tape 90128, AR tape 90469, AR tape 90368, AR tape 90119, AR tape 92276, and AR tape 90741 (Adhesives Research, Inc., Glen Rock, Pa.). Examples of hydrophilic film include, but are not limited to, Vistex® and Visguard® films from (Film Specialties Inc., Hillsborough, N.J.), and Lexan HPFAF (GE Plastics, Pittsfield, Mass.). Other hydrophilic surfaces are available from Surmodics, Inc. (Eden Prairie, Minn.), Biocoat Inc. (Horsham, Pa.), Advanced Surface Technology (Billerica, Mass.), and Hydromer, Inc. (Branchburg, N.J.) and any combination thereof. Surfactants can be mixed with reaction polymers such as polyurethanes and epoxies to serve as a hydrophilic coating.

In some embodiments, the suitable material can be composed of or include a conductive and/or magnetic material. Conductive materials include, without limitation, metals, electrolytes, superconductors, semiconductors and some nonmetallic conductors such as graphite and conductive polymers. Magnetic materials include without limitation, any magnetic material including those that are ferromagnetic, paramagnetic and diamagnetic. In some embodiments, the magnetic material can include those that are electromagnetic (i.e., those materials that become magnetic or become a more powerful magnet when an electric current is applied to them). Exemplary magnetic materials include, but are not limited to, iron, nickel, cobalt, steel, rare earth metals (e.g., gadolinium, samarium, and neodymium), and combinations thereof.

In some embodiments, the suitable material can be composed or include an electric insulator material. Exemplary electric insulator materials include, but are not limited to, rubber, glass, oil, air, diamond, dry wood, dry cotton, plastic, fiberglass, porcelain, ceramics and quartz.

In some embodiments, the surface of the substrate is made out of the same material as the substrate and is essentially integrated and indistinguishable from the substrate. In some embodiments, the surface is made out of a different material as the substrate. In some embodiments the surface is essentially a coating, film, or layer present on at least part of or the entirety of the substrate and is thus readily distinguishable from the substrate.

### Array Features

As previously described the array can have one or more features. In some embodiments, one or more of the features can have sub-features. In some embodiments as previously described, the sub features themselves can form an array within the feature (or also referred to herein as a sub array). In some embodiments, number of features can range from 1 to 100, 1,000, 10,000, 100,000, 1,000,000 or more. In some embodiments, the number of features is 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000, 100,000, 1,000,000 or more.

The number of sub features can range from 1 to 100, 1,000, 10,000 or more. In some embodiments, the number of sub features is 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 15000, 20000, 25000, 30000, 35000, 40000, 45000, 50000, 100,000, 1,000,000 or more.

In some embodiments the features and/or sub features can be wells (including but not limited to, microwells, nanowells, picowells, etc.), capillaries, microcapillaries, nanocapillaries, droplets, beads, oligonucleotides, polynucleotides, antibodies, affibodies, aptamers, polypeptide:polynucleotide complexes, gel forms, hydrogel forms, columns, matrices, and any permissible combinations thereof. In some embodiments, the features and/or subfeatures are spatial barcodes.

In some embodiments the features and/or sub features can hold a volume ranging from 1-1,000 pm3, nm3, μm3, cm3, mm3, or L3. In some embodiments, the wells, microwells, and/or nanowells capillaries, microcapillaries, nanocapillaries, and/or other areas formed on a surface of a substrate can hold a volume can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm3, nm3, μm3, cm3, mm3, or L3.

In some embodiments, one or more dimensions of the features and/or sub features (e.g., a length, a width, a height, a diameter, and/or the like) ranges from about 1-1,000 pm, nm, μm, cm, or mm. In some embodiments, one or more dimensions of the surface is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the largest dimension of the features and/or sub features ranges from 1-1,000 pm, nm, μm, cm, or mm. In some embodiments, the largest dimension of the surface is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the smallest dimension of the features and/or sub features ranges from about 1-1,000 pm, nm, μm, cm, or mm. In some embodiments, the smallest dimension of the surface is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

The features can be any container, region, area, droplet, vessel, and the like capable of containing a volume of fluid. In some of such embodiments, the features are wells, (including but not limited to, microwells, nanowells, picowells, etc.) capillaries, microcapillaries, nanocapillaries, and/or other areas formed on a surface of a substrate. The wells, microwells, and/or nanowells capillaries, microcapillaries, nanocapillaries, and/or other areas formed on a surface can be any regular or irregular 2D or 3D shape. In some embodiments, all the wells, microwells, and/or nanowells capillaries, microcapillaries, nanocapillaries, and/or other areas formed on a surface are homogenous. In some embodiments, all the wells, microwells, and/or nanowells capillaries, microcapillaries, nanocapillaries, and/or other areas formed on a surface are heterogenous.

In some embodiments the features, (e.g., wells, capillaries, microcapillaries, nanocapillaries, and/or other areas formed on a surface of a substrate can have a surface capable of holding a fluid) can include or be composed of a cell scaffold material and/or a material that facilitates cell adherence to a surface. Exemplary cell scaffold materials include, but are not limited to Matrigel, collagen and other extracellular matrix components, decellularized tissue, polysaccharides (e.g., alginate, chitosan, cellulose, dextran, chitin, glycosaminoglycan, hyaluronic acid, agarose and combinations thereof), polymers, ceramics, any material set forth in Nikolova and Chavali (2019. Bioact Mater. 4:271-292), particularly e.g., Tables 1-3 and Sections 3-6, and any combination thereof.

### Droplets

In some embodiments the spatial barcodes are loaded into droplets. In a preferred embodiment, the oligonucleotide spatial barcodes can be produced in a droplet PCR approach without the use of beads, as described in Redin, et al. Efficient whole genome haplotyping and high-throughput single molecule phasing with barcode-linked reads (2018) doi:10.1101/356121. Droplet formation can be achieved utilizing commercially available devices for droplet generation. One preferred method of droplet generation can be achieved by emulsion droplets formed by simple shaking. Redin, et al. Efficient whole genome haplotyping and high-throughput single molecule phasing with barcode-linked reads (2018) doi:10.1101/356121; Redin et al. Nucl. Acid Res. 45:13 (2017 doi: 10.1093/nar/gkx436, at ‘Emulsion Reactions’, incorporated herein by reference. Advantageously, the droplet formation approach allows droplet production with use of non-proprietary systems.

Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions, e.g., a library may contain plurality of different compounds at a range of concentrations encapsulated as separate library elements for screening their effect on cells or enzymes, alternatively a library could be composed of a plurality of different primer pairs encapsulated as different library elements for targeted amplification of a collection of loci, alternatively a library could contain a plurality of different antibody species encapsulated as different library elements to perform a plurality of binding assays. The introduction of a library of reaction conditions onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second. However, in practice droplets pack with oil between them that slowly drains. Over time the carrier fluid drains from the library droplets and the number density of the droplets (number/mL) increases. Hence, a simple fixed rate of infusion for the drive fluid does not provide a uniform rate of introduction of the droplets into the microfluidic channel in the substrate. Moreover, library-to-library variations in the mean library droplet volume result in a shift in the frequency of droplet introduction at the confluence point. Thus, the lack of uniformity of droplets that results from sample variation and oil drainage provides another problem to be solved. For example, if the nominal droplet volume is expected to be 10 pico-liters in the library, but varies from 9 to 11 pico-liters from library-to-library then a 10,000 pico-liter/second infusion rate will nominally produce a range in frequencies from 900 to 1,100 droplet per second. In short, sample to sample variation in the composition of dispersed phase for droplets made on chip, a tendency for the number density of library droplets to increase over time and library-to-library variations in mean droplet volume severely limit the extent to which frequencies of droplets may be reliably matched at a confluence by simply using fixed infusion rates. In addition, these limitations also have an impact on the extent to which volumes may be reproducibly combined. Combined with typical variations in pump flow rate precision and variations in channel dimensions, systems are severely limited without a means to compensate on a run-to-run basis. The foregoing facts not only illustrate a problem to be solved, but also demonstrate a need for a method of instantaneous regulation of microfluidic control over microdroplets within a microfluidic channel. Combinations of surfactant(s) and oils must be developed to facilitate generation, storage, and manipulation of droplets to maintain the unique chemical/biochemical/biological environment within each droplet of a diverse library. Therefore, the surfactant and oil combination must (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition to the requirements on the droplet library function and stability, the surfactant-in-oil solution must be coupled with the fluid physics and materials associated with the platform. Specifically, the oil solution must not swell, dissolve, or degrade the materials used to construct the microfluidic chip, and the physical properties of the oil (e.g., viscosity, boiling point, etc.) must be suited for the flow and operating conditions of the platform. Droplets formed in oil without surfactant are not stable to permit coalescence, so surfactants must be dissolved in the oil that is used as the continuous phase for the emulsion library. Surfactant molecules are amphiphilic--part of the molecule is oil soluble, and part of the molecule is water soluble. When a water-oil interface is formed at the nozzle of a microfluidic chip for example in the inlet module described herein, surfactant molecules that are dissolved in the oil phase adsorb to the interface. The hydrophilic portion of the molecule resides inside the droplet and the fluorophilic portion of the molecule decorates the exterior of the droplet. The surface tension of a droplet is reduced when the interface is populated with surfactant, so the stability of an emulsion is improved. In addition to stabilizing the droplets against coalescence, the surfactant should be inert to the contents of each droplet and the surfactant should not promote transport of encapsulated components to the oil or other droplets. A droplet library may be made up of a number of library elements that are pooled together in a single collection (see, e.g., US Patent Publication No. 2010002241). Libraries may vary in complexity from a single library element to 1015 library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” are also referred to herein as an “emulsion library” or “emulsion libraries.” These terms are used interchangeably throughout the specification. A cell library element may include, but is not limited to, hybridomas, B-cells, primary cells, cultured cell lines, cancer cells, stem cells, cells obtained from tissue, or any other cell type. Cellular library elements are prepared by encapsulating a number of cells from one to hundreds of thousands in individual droplets. The number of cells encapsulated is usually given by Poisson statistics from the number density of cells and volume of the droplet. However, in some cases the number deviates from Poisson statistics as described in Edd et al., “Controlled encapsulation of single-cells into monodisperse picolitre drops.” Lab Chip, 8(8): 1262-1264, 2008. The discrete nature of cells allows for libraries to be prepared in mass with a plurality of cellular variants all present in a single starting media and then that media is broken up into individual droplet capsules that contain at most one cell. These individual droplets capsules are then combined or pooled to form a library consisting of unique library elements. Cell division subsequent to, or in some embodiments following, encapsulation produces a clonal library element. A bead based library element may contain one or more beads of a given type and may also contain other reagents, such as antibodies, enzymes or other proteins. In the case where all library elements contain different types of beads, but the same surrounding media, the library elements may all be prepared from a single starting fluid or have a variety of starting fluids. In the case of cellular libraries prepared in mass from a collection of variants, such as genomically modified, yeast or bacteria cells, the library elements will be prepared from a variety of starting fluids. Often it is desirable to have exactly one cell per droplet with only a few droplets containing more than one cell when starting with a plurality of cells or yeast or bacteria, engineered to produce variants on a protein. In some cases, variations from Poisson statistics may be achieved to provide an enhanced loading of droplets such that there are more droplets with exactly one cell per droplet and few exceptions of empty droplets or droplets containing more than one cell. Examples of droplet libraries are collections of droplets that have different contents, ranging from beads, cells, small molecules, DNA, primers, antibodies. Smaller droplets may be in the order of femtoliter (fL) volume drops, which are especially contemplated with the droplet dispensers. The volume may range from about 5 to about 600 fL. The larger droplets range in size from roughly 0.5 micron to 500 micron in diameter, which corresponds to about 1 picoliter to 1 nano liter. However, droplets may be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges. The droplets within the emulsion libraries of the present invention may be contained within an immiscible oil which may comprise at least one fluorosurfactant. In some embodiments, the fluorosurfactant within the immiscible fluorocarbon oil may be a block copolymer consisting of one or more perfluorinated polyether (PFPE) blocks and one or more polyethylene glycol (PEG) blocks. In other embodiments, the fluorosurfactant is a triblock copolymer consisting of a PEG center block covalently bound to two PFPE blocks by amide linking groups. The presence of the fluorosurfactant (similar to uniform size of the droplets in the library) is critical to maintain the stability and integrity of the droplets and is also essential for the subsequent use of the droplets within the library for the various biological and chemical assays described herein. Fluids (e.g., aqueous fluids, immiscible oils, etc.) and other surfactants that may be utilized in the droplet libraries of the present invention are described in greater detail herein. The present invention can accordingly involve an emulsion library which may comprise a plurality of aqueous droplets within an immiscible oil (e.g., fluorocarbon oil) which may comprise at least one fluorosurfactant, wherein each droplet is uniform in size and may comprise the same aqueous fluid and may comprise a different library element. The present invention also provides a method for forming the emulsion library which may comprise providing a single aqueous fluid which may comprise different library elements, encapsulating each library element into an aqueous droplet within an immiscible fluorocarbon oil which may comprise at least one fluorosurfactant, wherein each droplet is uniform in size and may comprise the same aqueous fluid and may comprise a different library element, and pooling the aqueous droplets within an immiscible fluorocarbon oil which may comprise at least one fluorosurfactant, thereby forming an emulsion library. For example, in one type of emulsion library, all different types of elements (e.g., cells or beads), may be pooled in a single source contained in the same medium. After the initial pooling, the cells or beads are then encapsulated in droplets to generate a library of droplets wherein each droplet with a different type of bead or cell is a different library element. The dilution of the initial solution enables the encapsulation process. In some embodiments, the droplets formed will either contain a single cell or bead or will not contain anything, i.e., be empty. In other embodiments, the droplets formed will contain multiple copies of a library element. The cells or beads being encapsulated are generally variants on the same type of cell or bead. In another example, the emulsion library may comprise a plurality of aqueous droplets within an immiscible fluorocarbon oil, wherein a single molecule may be encapsulated, such that there is a single molecule contained within a droplet for every 20-60 droplets produced (e.g., 20, 25, 30, 35, 40, 45, 50, 55, 60 droplets, or any integer in between). Single molecules may be encapsulated by diluting the solution containing the molecules to such a low concentration that the encapsulation of single molecules is enabled. Formation of these libraries may rely on limiting dilutions.

The present invention also provides an emulsion library which may comprise at least a first aqueous droplet and at least a second aqueous droplet within an oil, in one embodiment a fluorocarbon oil, which may comprise at least one surfactant, in one embodiment a fluorosurfactant, wherein the at least first and the at least second droplets are uniform in size and comprise a different aqueous fluid and a different library element. The present invention also provides a method for forming the emulsion library which may comprise providing at least a first aqueous fluid which may comprise at least a first library of elements, providing at least a second aqueous fluid which may comprise at least a second library of elements, encapsulating each element of said at least first library into at least a first aqueous droplet within an immiscible fluorocarbon oil which may comprise at least one fluorosurfactant, encapsulating each element of said at least second library into at least a second aqueous droplet within an immiscible fluorocarbon oil which may comprise at least one fluorosurfactant, wherein the at least first and the at least second droplets are uniform in size and may comprise a different aqueous fluid and a different library element, and pooling the at least first aqueous droplet and the at least second aqueous droplet within an immiscible fluorocarbon oil which may comprise at least one fluorosurfactant thereby forming an emulsion library. One of skill in the art will recognize that methods and systems of the invention need not be limited to any particular type of sample, and methods and systems of the invention may be used with any type of organic, inorganic, or biological molecule (see, e.g., US Patent Publication No. 20120122714).

### Capture and Recognition Molecules

Capture molecules (also referred to interchangeably herein as recognition molecules, targeting moieties, targeting molecules, capture probe and the like) and include molecules such as ligands, receptors, aptamers, DNA segments, enzymes, antigens, antibodies, tailored for the molecules of interest. In embodiments, the capture molecule comprises a sequence specific for a target molecule of interest, a sequence specific for capture of an SNP, sequence specific for drug resistance or cancer markers, a Tn5 sequence, a 16S sequence, a poly(d)T sequence, a random hexamer sequence, a trypsin molecule, an antibody, a Protein Epitope Signature Tag (PrEST) sequence, or a combination thereof.

In some embodiments, the spatial barcodes further comprise a capture molecule or moiety. In some embodiments the spatial barcodes can be included in a recognition molecule barcode, array probe, and/or array bead probe. The spatial barcodes can also comprise one or more of a ligation sequence, a priming sequence, and a unique sequence. In particular embodiments, one or more guide RNAs, or one or more CRISPR systems comprising a guide polynucleotide and a nucleotide sequence encoding a Cas protein or can be appended or linked to the spatial barcodes. Advantageously, the oligonucleotides can be of any desired length, including lengths of 10 to about 400 nucleotides. A ligation sequence is a sequence complementary to a second nucleotide sequence which allows for ligation of the spatial barcode to another entity comprising the second nucleotide sequence, e.g., another detectable oligonucleotide tag or an oligonucleotide adapter. A priming sequence is a sequence complementary to a primer, e.g., an oligonucleotide primer used for an amplification reaction such as but not limited to PCR.

In some embodiments, a capture molecule (or recognition molecule) includes a recognition molecule barcode. (See e.g., FIG. 27). As exemplified in FIG. 27, a recognition molecule (e.g., an antibody) is coupled to a barcode (e.g., a DNA barcode) specific to that recognition molecule (e.g., antibody). The recognition molecule barcode also contains a region that is a target for a capture molecule on an array probe (that contains a spatial barcode) (see e.g., FIG. 26). Thus, in some embodiments, more than one capture/recognition molecule can be utilized in connection with different components of the assay.

In some embodiments, a capture molecule (or recognition molecule) does not include or is not coupled to a capture molecule or recognition molecule barcode. See e.g., FIGS. 39-43 and 62 and related discussions. In these embodiments, the non-barcoded capture molecule can bind to a target and provide spatial information on the target (e.g., such as spatial proteomic information), that can be correlated to spatial transcriptional information as described and with respect to e.g., FIGS. 39-43 and 62, such as via one or more imaging techniques, spatial transcriptomic library analysis techniques, or a combination thereof. In some embodiments, both spatial barcoded and non-spatial barcoded capture molecules are used and can provide transcriptomic information, proteomic information, spatial transcriptomic information, spatial proteomic information, or a combination thereof of the sample. It will be appreciated that in these embodiments, different types of information can be obtained for different targets. In some embodiments, one or more of the targets can be an assay control.

The capture molecule can comprise any other entity capable of binding to the capture sequence, e.g., an antibody or peptide. An index sequence is a sequence comprising a unique nucleotide sequence and/or a detectable moiety as described above. A capture entity can therefore be any molecule capable of attaching and/or binding to a nucleic acid (i.e., for example, a barcode nucleic acid). For example, a capture molecule may be an oligonucleotide attached to a bead, wherein the oligonucleotide is at least partially complementary to another oligonucleotide. A capture probe may comprise a polyethylene glycol linker, an antibody, a polyclonal antibody, a monoclonal antibody, a Fab fragment, a biological receptor complex, an enzyme, a hormone, an antigen, and/or a fragment or portion thereof. The capture probe can further comprise additional adaptors for use in further processing, for example a flow cell sequence for use with flow cell technologies such as those manufactured by Illumina.

### Decoding

While in some embodiments, the spatial barcodes are known at the time of deposition, in other instances decoding of the spatial barcode is necessary. As an example, the spatial barcodes of each location can be known at the step of depositing because conductivity-coded beads are specific to pre-etched wells of the substrate, and the wells accept only a bead of a specific charge. In this instance, the pre-etched wells provide an x,y coordinate. In other instances, the spatial barcode may need a step of decoding the barcode deposited. One example of decoding may arise when the spatial barcode comprises an oligonucleotide sequence that requires sequencing.

The step of decoding can comprise sequential hybridization, in-situ sequencing, laser scanning of color-coded, beads, DNA microscopy, camera systems for color-coded beads, and other imaging systems as needed. Decoding may also comprise Voronoi tessellation and sequence similarity. For example, if using FACS, FACS would decode the spatial barcode carried based on the color scheme of the bead, and further use of a camera system can track the location where each bead is deposited on the solid substrate. Sequential hybridization techniques such as Illumina, seqFISH or MERFISH technologies can be utilized for decoding the spatial barcodes.

### Depositing the Sample and Capturing Material of Interest

Depositing the sample can, in some embodiments include fixation of the sample to the solid substrate. In embodiments, the sample is tissue, which in embodiments is living. Living tissue can include use of processes as described in Nat Methods. 2014 Feb.; 11(2):190-196 doi: 10.1038/nmeth.2804 to maintain the tissue as living.

In some cases, the cells, organelle, and/or nuclei may be permeabilized to allow access for nucleic acid processing reagents. The permeabilization may be performed in a way to minimally perturb the cells, organelles, and/or nuclei. In embodiments, permeabilization steps, including pre-permeabilization are automated. In some instances, the cells may be permeabilized using a permeabilization agent. Examples of permeabilization agents include NP40, digitonin, tween, streptolysin, exonuclease 1 buffer (NEB) and pepsin, and cationic lipids. In other instances, the cells, organelles, and/or nuclei may be permeabilized using hypotonic shock and/or ultrasonication. In other cases, the nucleic acid processing reagents e.g., enzymes such as insertional enzyme, may be highly charged, which may allow them to permeabilize through the membranes of the cells, organelles, or nuclei. In certain examples, the methods include permeabilizing nuclei. Other embodiments include use of cell penetrating peptides to deliver cargo to the cell and allow capture of material.

Tissue can be reduced in size using methods as discussed, for example in Nature Methods volume 13, pages 859-867 (2016), which provides preservation of intact organ tissue while reducing size by over 50%, incorporated herein by reference.

Capture of material will depend on the type of capture molecule used as well as permeabilization technique. In some embodiments, the capture material is a nucleic acid. In some embodiments, the permeabilization of the tissue allows for release of contents of target molecules of interest that are captured by the capture moiety. In embodiments, the process is as described in Stahl et al. (22), incorporated herein by reference.

### Correlating Captured Material to a Position in the Sample on the Solid Substrate

Correlating the captured material to a position in the sample on the solid substrate may include decoding the spatial barcode, as described herein. The spatial barcode provides information for the position of the captured material on the solid substrate. Correlating the position in the sample on the solid substrate can also include use of the spatial barcode as the x,y coordinates as well as use of additional information for z coordinate, which indicates location in the volume of the sample on the solid substrate. In embodiments, the z coordinate is identified by staining the sample. In other embodiments, the z coordinate is identified using a CRISPR system comprising different guide molecules. Regardless of the method used to designate the z coordinate, correlation of the z coordinate and the x,y coordinate encoded by the spatial barcode is performed.

In embodiments, the sample is stained, and an image is captured of the sample. In embodiments, the morphology of the stained sample is recorded by the image, and further annotating of regions of the stained sample is performed. In embodiments, the image is assigned pixel coordinates that correspond to the centroids of each x,y area of the solid substrate. The pixel coordinates of the image can then be correlated to the x,y coordinates of a location on the solid substrate. The number of pixels assigned to the images can correlate in some instances to the centroids of each microwell on an array, or to the center of each spot or dot on a nanodot array. Accordingly, a higher number of spots spaced more closely together will result in a higher number of pixels assigned to an image, and a higher density analysis of the sample.

### Assigning a Cell Type or Subtype

Assigning a cell type to a cell subpopulation in the sample can be based on evaluation of the capture molecules at a particular position on the solid substrate. Cell surface molecules, differential gene expression signatures, and presence or absence of moieties can be utilized in assigning a cell type to a cell in the sample.

Cell type assignments can include correlating gene expression between one or more replicates of measured data and bulk RNA sequencing data. Numbers of shared or present genes can be evaluated between the datasets. Proteome sensing can be performed simultaneously with transcriptome evaluation. The integration of the output from automated imaging of stained tissues or cells with the output of gene-by-barcode expression can provide an output for assigning cell type or subtype.

In embodiments, generating cell type-specific gene signatures includes correlating gene expression levels or protein expression levels to cell type prediction scores, and then considering the most highly correlated genes. In embodiments, assigning a cell type or cell subtype includes the automated processing of imaging, single-cell sequencing, and/or proteome, transcriptome or spatial information to assign cell types and subtypes.

### Single Cell Sequencing and Other Sequencing Techniques

The methods herein may further include sequencing one or more nucleic acids processed by the steps herein. For example, after barcoded and isolated, the genomic DNA, cDNA, the barcode sequence(s), and a portion thereof, may be sequenced. One or more steps of in situ sequencing can be automated, as detailed elsewhere herein, including in Example 3.

In some cases, the sequencing may be next generation sequencing. The terms “next-generation sequencing” or “high-throughput sequencing” refer to the so-called parallelized sequencing-by-synthesis or sequencing-by-ligation platforms currently employed by Illumina, Life Technologies, and Roche, etc. Next-generation sequencing methods may also include nanopore sequencing methods or electronic-detection based methods such as Ion Torrent technology commercialized by Life Technologies or single-molecule fluorescence-based method commercialized by Pacific Biosciences. Any method of sequencing known in the art can be used before and after isolation. In certain embodiments, a sequencing library is generated and sequenced.

At least a part of the processed nucleic acids and/or barcodes attached thereto may be sequenced to produce a plurality of sequence reads. The fragments may be sequenced using any convenient method. For example, the fragments may be sequenced using Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol Biol. 2009; 553:79-108); Appleby et al (Methods Mol Biol. 2009; 513:19-39) and Morozova et al (Genomics. 2008 92:255-64), which are incorporated by reference for the general descriptions of the methods and the particular steps of the methods, including all starting products, methods for library preparation, reagents, and final products for each of the steps. As would be apparent, forward and reverse sequencing primer sites that are compatible with a selected next generation sequencing platform can be added to the ends of the fragments during the amplification step. In certain embodiments, the fragments may be amplified using PCR primers that hybridize to the tags that have been added to the fragments, where the primer used for PCR have 5′ tails that are compatible with a particular sequencing platform. In certain cases, the primers used may contain a molecular barcode (an “index”) so that different pools can be pooled together before sequencing, and the sequence reads can be traced to a particular sample using the barcode sequence.

In some cases, the sequencing may be performed at certain “depth.” The terms “depth” or “coverage” as used herein refers to the number of times a nucleotide is read during the sequencing process. In regards to single cell RNA sequencing, “depth” or “coverage” as used herein refers to the number of mapped reads per cell. Depth—In regards to genome sequencing may be calculated from the length of the original genome (G), the number of reads(N), and the average read length(L) as N×L/G. For example, a hypothetical genome with 2,000 base pairs reconstructed from 8 reads with an average length of 500 nucleotides will have 2× redundancy.

In some cases, the sequencing herein may be low-pass sequencing. The terms “low-pass sequencing” or “shallow sequencing” as used herein refers to a wide range of depths greater than or equal to 0.1× up to 1×. Shallow sequencing may also refer to about 5000 reads per cell (e.g., 1,000 to 10,000 reads per cell).

In some cases, the sequencing herein can be deep sequencing or ultra-deep sequencing. The term “deep sequencing” as used herein indicates that the total number of reads is many times larger than the length of the sequence under study. The term “deep” as used herein refers to a wide range of depths greater than 1× up to 100×. Deep sequencing may also refer to 100× coverage as compared to shallow sequencing (e.g., 100,000 to 1,000,000 reads per cell). The term “ultra-deep” as used herein refers to higher coverage (>100-fold), which allows for detection of sequence variants in mixed populations.

Multiple technologies have been described that massively parallelize the generation of single cell RNA seq libraries that can be used in the present disclosure. As used herein, RNA-seq methods refer to high-throughput single-cell RNA-sequencing protocols. RNA-seq includes, but is not limited to, Drop-seq, Seq-Well, InDrop and 1Cell Bio. RNA-seq methods also include, but are not limited to, smart-seq2, TruSeq, CEL-Seq, STRT, ChIRP-Seq, GRO-Seq, CLIP-Seq, Quartz-Seq, or any other similar method known in the art (see, e.g., “Sequencing Methods Review” Illumina® Technology, Sequencing Methods Review available at illumina.com.

In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 Oct.; 14(10):955-958; and International patent application number PCT/US2016/059239, published as WO2017164936 on Sep. 28, 2017, which are herein incorporated by reference in their entirety.

The term “tagmentation” refers to a step in the Assay for Transposase Accessible Chromatin using sequencing (ATAC-seq) as described. (See, Buenrostro, J. D., Giresi, P. G., Zaba, L. C., Chang, H. Y., Greenleaf, W. J., Transposition of native chromatin for fast and sensitive epigenomic profiling of open chromatin, DNA-binding proteins and nucleosome position. Nature methods 2013; 10 (12): 1213-1218). Specifically, a hyperactive Tn5 transposase loaded in vitro with adapters for high-throughput DNA sequencing, can simultaneously fragment and tag a genome with sequencing adapters. In one embodiment the adapters are compatible with the methods described herein.

In certain embodiments, tagmentation is used to introduce adaptor sequences to genomic DNA in regions of accessible chromatin (e.g., between individual nucleosomes) (see, e.g., US20160208323A1; US20160060691A1; WO2017156336A1; and Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7). In certain embodiments, tagmentation is applied to bulk samples or to single cells in discrete volumes.

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

Drop-sequence methods or Drop-seq are contemplated for the present invention. Cells come in different types, sub-types and activity states, which are classify based on their shape, location, function, or molecular profiles, such as the set of RNAs that they express. RNA profiling is in principle particularly informative, as cells express thousands of different RNAs. Approaches that measure for example the level of every type of RNA have until recently been applied to “homogenized” samples—in which the contents of all the cells are mixed together. Methods to profile the RNA content of tens and hundreds of thousands of individual human cells have been recently developed, including from brain tissues, quickly and inexpensively. To do so, special microfluidic devices have been developed to encapsulate each cell in an individual drop, associate the RNA of each cell with a barcode unique to that cell/drop, measure the expression level of each RNA with sequencing, and then use the cell barcodes to determine which cell each RNA molecule came from. See, e.g., methods of Macosko et al., 2015, Cell 161, 1202-1214 and Klein et al., 2015, Cell 161, 1187-1201 are contemplated for the present invention.

In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 Oct.; 14(10):955-958; and International patent application number PCT/US2016/059239, published as WO2017164936 on Sep. 28, 2017, which are herein incorporated by reference in their entirety.

Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. Furthermore, incorporation of microfluidics technology enhances system integration and automation. Microfluidic reactions are generally conducted in microdroplets or microwells. The ability to conduct reactions in microdroplets depends on being able to merge different sample fluids and different microdroplets. See, e.g., US Patent Publication No. 20120219947. See also international patent application serial no. PCT/US2014/058637 for disclosure regarding a microfluidic laboratory on a chip.

Droplet/microwell microfluidics offers significant advantages for performing high-throughput screens and sensitive assays. Droplets allow sample volumes to be significantly reduced, leading to concomitant reductions in cost. Manipulation and measurement at kilohertz speeds enable up to 108 discrete biological entities (including, but not limited to, individual cells or organelles) to be screened in a single day. Compartmentalization in droplets increases assay sensitivity by increasing the effective concentration of rare species and decreasing the time required to reach detection thresholds. Droplet microfluidics combines these powerful features to enable currently inaccessible high-throughput screening applications, including single-cell and single-molecule assays. See, e.g., Guo et al., Lab Chip, 2012, 12, 2146-2155.

Drop-Sequence methods and apparatus provides a high-throughput single-cell RNA-Seq and/or targeted nucleic acid profiling (for example, sequencing, quantitative reverse transcription polymerase chain reaction, and the like) where the RNAs from different cells are tagged individually, allowing a single library to be created while retaining the cell identity of each read. A combination of molecular barcoding and emulsion-based microfluidics to isolate, lyse, barcode, and prepare nucleic acids from individual cells in high-throughput is used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode ˜10,000-100,000 cells.

InDrop™, also known as in-drop seq, involves a high-throughput droplet-microfluidic approach for barcoding the RNA from thousands of individual cells for subsequent analysis by next-generation sequencing (see, e.g., Klein et al., Cell 161(5), pp 1187-1201, 21 May 2015). Specifically, in in-drop seq, one may use a high diversity library of barcoded primers to uniquely tag all DNA that originated from the same single cell. Alternatively, one may perform all steps in drop.

Well-based biological analysis or Seq-Well is also contemplated for the present invention. The well-based biological analysis platform, also referred to as Seq-well, facilitates the creation of barcoded single-cell sequencing libraries from thousands of single cells using a device that contains 100,000 40-micron wells. Importantly, single beads can be loaded into each microwell with a low frequency of duplicates due to size exclusion (average bead diameter 35 μm). By using a microwell array, loading efficiency is greatly increased compared to drop-seq, which requires poison loading of beads to avoid duplication at the expense of increased cell input requirements. Seq-well, however, is capable of capturing nearly 100% of cells applied to the surface of the device.

Seq-well is a methodology which allows attachment of a porous membrane to a container in conditions which are benign to living cells. Combined with arrays of picoliter-scale volume containers made, for example, in PDMS, the platform provides the creation of hundreds of thousands of isolated dialysis chambers which can be used for many different applications. The platform also provides single cell lysis procedures for single cell RNA-seq, whole genome amplification or proteome capture; highly multiplexed single cell nucleic acid preparation (˜100× increase over current approaches); highly parallel growth of clonal bacterial populations thus providing synthetic biology applications as well as basic recombinant protein expression; selection of bacterial that have increased secretion of a recombinant product possible product could also be small molecule metabolite which could have considerable utility in chemical industry and biofuels; retention of cells during multiple microengraving events; long term capture of secreted products from single cells; and screening of cellular events. Principles of the present methodology allow for addition and subtraction of materials from the containers, which has not previously been available on the present scale in other modalities.

Seq-Well also enables stable attachment (through multiple established chemistries) of porous membranes to PDMS nanowell devices in conditions that do not affect cells. Based on requirements for downstream assays, amines are functionalized to the PDMS device and oxidized to the membrane with plasma. With regard to general cell culture uses, the PDMS is amine functionalized by air plasma treatment followed by submersion in an aqueous solution of poly(lysine) followed by baking at 80° C. For processes that require robust denaturing conditions, the amine must be covalently linked to the surface. This is accomplished by treating the PDMS with air plasma, followed by submersion in an ethanol solution of amine-silane, followed by baking at 80° C., followed by submersion in 0.2% phenylene diisothiocyanate (PDITC) DMF/pyridine solution, followed by baking, followed by submersion in chitosan or poly(lysine) solution. For functionalization of the membrane for protein capture, membrane can be amine-silanized using vapor deposition and then treated in solution with NHS-biotin or NHS-maleimide to turn the amine groups into the crosslinking species.

After functionalization, the device is loaded with cells (bacterial, mammalian or yeast) in compatible buffers. The cell-laden device is then brought in contact with the functionalized membrane using a clamping device. A plain glass slide is placed on top of the membrane in the clamp to provide force for bringing the two surfaces together. After an hour incubation, as one hour is a preferred time span, the clamp is opened and the glass slide is removed. The device can then be submerged in any aqueous buffer for days without the membrane detaching, enabling repetitive measurements of the cells without any cell loss. The covalently-linked membrane is stable in many harsh buffers including guanidine hydrochloride which can be used to robustly lyse cells. If the pore size of the membrane is small, the products from the lysed cells will be retained in each well. The lysing buffer can be washed out and replaced with a different buffer which allows binding of biomolecules to probes preloaded in the wells. The membrane can then be removed, enabling addition of enzymes to reverse transcribe or amplify nucleic acids captured in the wells after lysis. Importantly, the chemistry enables removal of one membrane and replacement with a membrane with a different pore size to enable integration of multiple activities on the same array.

As discussed, while the platform has been optimized for the generation of individually barcoded single-cell sequencing libraries following confinement of cells and mRNA capture beads (Macosko, et al. Cell. 2015 May 21; 161(5): 1202-1214), it is capable of multiple levels of data acquisition. The platform is compatible with other assays and measurements performed with the same array. For example, profiling of human antibody responses by integrated single-cell analysis is discussed with regard to measuring levels of cell surface proteins (Ogunniyi, A. O., B. A. Thomas, T. J. Politano, N. Varadarajan, E. Landais, P. Poignard, B. D. Walker, D. S. Kwon, and J. C. Love, “Profiling Human Antibody Responses by Integrated Single-Cell Analysis” Vaccine, 32(24), 2866-2873.) The authors demonstrate a complete characterization of the antigen-specific B cells induced during infections or following vaccination, which enables and informs one of skill in the art how interventions shape protective humoral responses. Specifically, this disclosure combines single-cell profiling with on-chip image cytometry, microengraving, and single-cell RT-PCR. Similarly, upon release of barcoded nucleic acids from other applications, such barcoded molecules can be processed and used as libraries in the sequencing methods as disclosed herein.

### Use of Signature Genes

As used herein a “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g., blood samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g., cancer grade), or linked to a particular outcome or progression of the disease or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular tissue cell or tissue cell (sub)population or subcellular population if it is upregulated or only present, detected or detectable in that particular tissue cell, cell (sub)population, or subcellular population or alternatively is downregulated or only absent, or undetectable in that particular tissue cell or tissue cell (sub)population or subcellular population. In this context, a signature consists of one or more differentially expressed genes/proteins or differential epigenetic elements when comparing different cells or cell (sub)populations or subcellular populations, including comparing different tumor cells or tumor cell (sub)populations or tumor subcellular populations, as well as comparing tissue cells or tissue cell (sub)populations with other tissue types or tissue cell (sub)populations or subcellular populations, or tumor cells or tumor cell (sub)populations with non-tumor cells or non-tumor cell (sub)populations or non-tumor subcellular populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. In particular embodiments, genes with an expression fold change greater than 1.5 are utilized for analysis.

As discussed herein, differentially expressed genes/proteins, or differential epigenetic elements may be differentially expressed on a single cell level or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tissue cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized and is preferably characterized by the signature as discussed herein. A cell (sub)population as referred to herein may constitute of a (sub)population of cells of a particular cell type characterized by a specific cell state. A subcellular population includes one or more of the structures within a cell, subcellular organisms or organelles, including Golgi apparatus, smooth+rough endoplasmic reticulum, nucleus and mitochondria.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

Signatures may be functionally validated as being uniquely associated with a particular immune responder phenotype. Induction or suppression of a particular signature may consequentially be associated with or causally drive a particular immune responder phenotype.

Various aspects and embodiments of the invention may involve analyzing gene signatures, protein signature, and/or other genetic or epigenetic signature based on single cell analyses (e.g., single cell RNA sequencing) or alternatively based on cell population analyses, as is defined herein elsewhere.

In further aspects, the invention relates to gene signatures, protein signature, and/or other genetic or epigenetic signature of particular tumor cell subpopulations, as defined herein elsewhere. The invention hereto also further relates to particular tumor cell subpopulations, which may be identified based on the methods according to the invention as discussed herein; as well as methods to obtain such cell (sub)populations and screening methods to identify agents capable of inducing or suppressing particular tumor cell (sub)populations.

The invention further relates to various uses of the gene signatures, protein signature, and/or other genetic or epigenetic signature as defined herein, as well as various uses of the tumor cells or tumor cell (sub)populations as defined herein. Particular advantageous uses include methods for identifying agents capable of inducing or suppressing particular tumor cell (sub)populations based on the gene signatures, protein signature, and/or other genetic or epigenetic signature as defined herein. The invention further relates to agents capable of inducing or suppressing particular tumor cell (sub)populations based on the gene signatures, protein signature, and/or other genetic or epigenetic signature as defined herein, as well as their use for modulating, such as inducing or repressing, a particular gene signature, protein signature, and/or other genetic or epigenetic signature. In one embodiment, genes in one population of cells may be activated or suppressed in order to affect the cells of another population. In related aspects, modulating, such as inducing or repressing, a particular a particular gene signature, protein signature, and/or other genetic or epigenetic signature may modify overall tumor composition, such as tumor cell composition, such as tumor cell subpopulation composition or distribution, or functionality.

The signature genes of the present invention can be derived from references identifying gene sets for particular types of tissue or cells. In embodiments, the tissue is from the central nervous system and the Allen Brain Atlas is used as a reference. Data from other published sources can be sued for reference, or from analysis of expression profiles of single-cells within a population of cells from freshly isolated samples of the same type. Overlaying single cell sequencing datasets with the spatial transcriptomics described herein allows for characterization of cell subtypes and their interactions within a three dimensional architecture that was previously poorly understood. The presence of subtypes may be determined by subtype specific signature genes. The presence of these specific cell types may be determined by applying the signature genes to bulk sequencing data in a sample. Not being bound by a theory, a tissue is a conglomeration of many cells that make up a tissue microenvironment, whereby the cells communicate and affect each other in specific ways. As such, specific cell types within this microenvironment may express signature genes specific for this microenvironment. Not being bound by a theory the signature genes of the present invention may be microenvironment specific, such as their expression in a tissue.

In certain examples, the methods can be used in tumors, in which not being bound by a theory, signature genes determined in single cells that originated in a tumor are specific to other tumors. Not being bound by a theory, a combination of cell subtypes in a tumor may indicate an outcome. Not being bound by a theory, the signature genes can be used to deconvolute the network of cells present in a tumor based on comparing them to data from bulk analysis of a tumor sample. Not being bound by a theory the presence of specific cells and cell subtypes may be indicative of tumor growth, invasiveness and resistance to treatment. The signature gene may indicate the presence of one particular cell type. The presence of cell types within a tumor may indicate that the tumor will be resistant to a treatment. In one embodiment, the signature genes of the present invention are applied to bulk sequencing data from a tumor sample obtained from a subject, such that information relating to disease outcome and personalized treatments is determined. In one embodiment, the novel signature genes are used to detect multiple cell states that occur in a subpopulation of tumor cells that are linked to resistance to targeted therapies and progressive tumor growth.

By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a negative control cell or than an average signal generated for the marker by a population of negative control cells, e.g., at least 2-fold, at least 4-fold, at least 10-fold, at least 20-fold, at least 30-fold, at least 40-fold, at least 50-fold higher or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of negative control cells. The upregulation and/or downregulation of gene or gene product, including the amount, may be included as part of the gene signature or expression profile.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR-), Youden index, or similar.

The signature genes utilized in the present invention can be discovered by analysis of expression profiles of single-cells within a population of cells from a similar sample or from previously published studies of the tissue or sample type, thus allowing the in-situ tissue profiling and transcriptomics described herein. The presence of subtypes may be determined by subtype specific signature gene, and the signature genes of the present invention may be microenvironment specific.

In one embodiment, the signature genes are detected by immunofluorescence, immunohistochemistry, fluorescence activated cell sorting (FACS), mass cytometry (CyTOF), drop-seq, RNA-seq, single cell qPCR, MERFISH (multiplex (in-situ) RNA FISH) and/or by in-situ hybridization. Other methods including absorbance assays and colorimetric assays are known in the art and may be used herein.

### Sample Types

Appropriate samples for use in the methods disclosed herein include any conventional biological sample obtained from an organism or a part thereof, such as a plant, animal, bacteria, and the like. In particular embodiments, the biological sample is obtained from an animal subject, such as a human subject. A biological sample is any solid or fluid sample obtained from, excreted by or secreted by any living organism, including, without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as an infection with a pathogenic microorganism, such as a pathogenic bacteria or virus). For example, a biological sample can be a biological fluid obtained from, for example, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will be appreciated that any method of obtaining tissue from a subject can be utilized, and that the selection of the method used will depend upon various factors such as the type of tissue, age of the subject, or procedures available to the practitioner. Standard techniques for acquisition of such samples are available in the art. See, for example Schluger et al., J. Exp. Med. 176:1327-33 (1992); Bigby et al., Am. Rev. Respir. Dis. 133:515-18 (1986); Kovacs et al., NEJM318:589-93 (1988); and Ognibene et al., Am. Rev. Respir. Dis. 129:929-32 (1984).

The tissue sample can advantageously be sourced from any organism e.g., plant, animal, bacterial or fungal. Samples may be a tissue sample, which can optionally be cultured, dead or living tissue. The array of the invention allows the capture of any nucleic acid, e.g., mRNA molecules, which are present in cells that are capable of transcription and/or translation. The arrays and methods of the invention are particularly suitable for isolating and analyzing the transcriptome or genome of cells within a sample, wherein spatial resolution of the transcriptomes or genomes is desirable, e.g., where the cells are interconnected or in contact directly with adjacent cells. However, it will be apparent to a person of skill in the art that the methods of the invention may also be useful for the analysis of the transcriptome or genome of different cells or cell types within a sample even if said cells do not interact directly, e.g., a blood sample. In other words, the cells do not need to present in the context of a tissue and can be applied to the array as single cells (e.g., cells isolated from a non-fixed tissue). Such single cells, whilst not necessarily fixed to a certain position in a tissue, are nonetheless applied to a certain position on the array and can be individually identified. Thus, in the context of analyzing cells that do not interact directly, or are not present in a tissue context, the spatial properties of the described methods may be applied to obtaining or retrieving unique or independent transcriptome or genome information from individual cells. Additionally, the simultaneous sensing of proteome and transcriptome can be performed on different cells or cell types within a sample utilizing the methods described herein.

The systems and methods as disclosed herein can be used to characterize tissues or cells from carcinomas or putative carcinomas.

In one aspect, the invention can evaluate, identify or quantify signature genes, gene products, and expression profiles of signature genes, gene networks, and gene products of tissues, tumors and/or component cells. The signature genes, gene products, and expression profiles are useful to identify components of tumors and tissues and states of such components, such as, without limitation, neoplastic cells, malignant cells, stem cells, immune cells, and malignant, microenvironmental, or immunologic states of such component cells.

The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumor including serous tumor, endometrioid tumor and mucinous cystadenocarcinoma, sex-cord-stromal tumor), prostate cancer, liver and bile duct carcinoma (e.g., hepatocellular carcinoma, cholangiocarcinoma, hemangioma), choriocarcinoma, seminoma, embryonal carcinoma, kidney cancer (e.g., renal cell carcinoma, clear cell carcinoma, Wilms tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, astrocytoma, medulloblastoma, craniopharyngioma, ependymoma, pinealoma, hemangioblastoma, acoustic neuroma, oligodendroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

In other embodiments, a sample may be an environmental sample, such as water, soil, or a surface such as industrial or medical surface. In some embodiments, methods such as disclosed in US patent publication No. 2013/0190196 may be applied for detection of nucleic acid signatures, specifically RNA levels, directly from crude cellular samples with a high degree of sensitivity and specificity. Sequences specific to each pathogen of interest may be identified or selected by comparing the coding sequences from the pathogen of interest to all coding sequences in other organisms by BLAST software.

As described herein, a sample for use with the invention may be a biological or environmental sample, such as a food sample (fresh fruits or vegetables, meats), a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or portability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In some embodiments, checking for food contamination by a virus that can be spread, in restaurants or other food providers; food surfaces; also checking food quality for manufacturers and regulators to determine the purity of meat sources; or identifying air or water contamination with pathogens.

A microbe in accordance with the invention may be a pathogenic microbe or a microbe that results in food or consumable product spoilage. A pathogenic microbe may be pathogenic or otherwise undesirable to humans, animals, or plants. For human or animal purposes, a microbe may cause a disease or result in illness. Animal or veterinary applications of the present invention may identify animals infected with a microbe. For example, the methods and systems of the invention may identify companion or farm animals with pathogens. In certain example embodiments, the virus may be any viral species that causes hemorrhagic fever, or other microbe causing similar symptoms.

In one embodiment, tumor cells are stained for cell subtype specific signature genes. In one embodiment, the cells are fixed. In another embodiment, the cells are formalin fixed and paraffin embedded. Tissue samples may also be fresh, fixed, or frozen. Not being bound by a theory, the presence of the cell subtypes in a tumor indicate outcome and personalized treatments. Not being bound by a theory, the cell subtypes may be quantitated in a section of a tumor and the number of cells indicates an outcome and personalized treatment.

In some embodiments, the sample is a tissue sample comprising a plurality of cells and can include one or more layers. The tissue sample size can be any suitable size, shape, or volume. In some embodiments, the tissue sample size can be about 1 to about 1000 nm², μm², cm², nm³, μm³, or cm³. Tissue samples can include any tissue or portion thereof obtained from an organism (e.g., a human, non-human animal, plant, fungi, etc.) or a product produced therefrom (e.g., an egg, fruit, nut, etc.). It will be appreciated that tissue samples can be obtained from living and non-living organisms. Food and feedstuffs produced from organisms are within the scope of “tissue samples” as used herein.

In some embodiments where the sample is a surface of an object or environment (table, ground, etc.), the sample can be optionally obtained using a suitable collection device (e.g., filter paper, etc.) that can maintain the spatial positioning of the cells relative to each other as they were on the surface of an object. The collection device can then be used similar to an intact tissue sample as described in the context of the present invention.

In some embodiments where the sample is a fluid sample (e.g., a biofluid or an environmental fluid (e.g., air, water, etc.), a sample collection device can be used to collect cells present in such fluid samples such that they become fixed in two dimensional or three dimensional space (e.g., filter paper, wax, hydrogels, and the like). The collection device can then be used similar to an intact tissue sample as described in the context of the present invention.

In some embodiments, the samples comprising a plurality of cells (including tissues) are cultured, expanded, modified (e.g., such as genetically modified), exposed to one or more test agents (e.g., pharmaceuticals, chemical agents, physical agents (e.g., heat, light, cold, pH, tension, etc.), or a combination thereof) prior to being analyzed by a method described herein. In some embodiments, the sample includes a plurality cells derived from or otherwise obtained from a biological or environmental sample, such as a tissue sample, fluid sample, or other sample as previously described. In some embodiments, the cells are cultured, expanded, modified (e.g., such as genetically modified), exposed to one or more test agents (e.g., pharmaceuticals, chemical agents, physical agents (e.g., heat, light, cold, pH, tension, etc.), or a combination thereof) prior to being analyzed by a method described herein.

Detection Based on rRNA Sequences

In certain example embodiments, the devices, systems, and methods disclosed herein may be used to distinguish multiple microbial species in a sample. In certain example embodiments, identification may be based on ribosomal RNA sequences, including the 16S, 23S, and 5S subunits. Methods for identifying relevant rRNA sequences are disclosed in U.S. Patent Application Publication No. 2017/0029872. In certain example embodiments, a set of guide RNA may be designed to distinguish each species by a variable region that is unique to each species or strain. Guide RNAs may also be designed to target RNA genes that distinguish microbes at the genus, family, order, class, phylum, kingdom levels, or a combination thereof. In certain example embodiments where amplification is used, a set of amplification primers may be designed to flanking constant regions of the ribosomal RNA sequence and a guide RNA designed to distinguish each species by a variable internal region. In certain example embodiments, the primers and guide RNAs may be designed to conserved and variable regions in the 16S subunit respectfully. Other genes or genomic regions that uniquely variable across species or a subset of species such as the RecA gene family, RNA polymerase β subunit, may be used as well. Other suitable phylogenetic markers, and methods for identifying the same, are discussed for example in Wu et al. arXiv:1307.8690 [q-bio.GN].

### Sample Staining

In some embodiments, the sample is stained. In a particularly preferred embodiment, the stain is hematoxylin and eosin (H&E) stain to prepare the sample for brightfield microscopy. With this method cell nuclei are stained blue and cytoplasm and many extra-cellular components in shades of pink. In histopathology many conditions can be diagnosed by examining an H&E alone. However sometimes additional information is required to provide a full differential diagnosis and this requires further, more specialized staining techniques. These may be “special stains” using dyes or metallic impregnations to define particular structures or microorganisms, or immuno-histochemical methods (IHC) involving the location of diagnostically useful proteins using labelled antibodies. Staining of the sample can allow identification of a molecule on the z axis.

Imaging and image analysis can advantageously be automated. In particular embodiments, a plurality of images can be captured prior to in situ reactions in the plurality of cells or tissue samples on the solid substrate. The plurality of captured images can be stitched together by the automated process described herein and detailed in Example 3. In particular embodiments, the segmented or stitched imaging can be integrated with information captured from spatial and single cell data. In embodiments, the correlating of a molecule to a position in the sample comprises further integrating the image data captured with the gene-by-barcode expression output

### Proteome Analysis

Proteome sensing may be performed both before and after spatially tagging the transcriptome of a sample. In an aspect, proteome analysis may be performed simultaneously with transcriptome analysis. In embodiments proteome sensing may comprise staining, for imaging according to embodiments detailed elsewhere herein. In exemplary embodiments, direct and secondary antibody fluorescent staining can be utilized to sense proteins in the tissue sample. Embodiments may comprise DNA-barcode antibodies, lipid-barcode antibodies or metal tagging can be utilized. In instances where DNA-barcode antibodies or lipid-barcode antibodies are utilized, the barcodes can be read out by methods known in the art, including in situ PCR, in situ qPCR, in situ sequencing, FISH/smFISH, sequential hybridization. In instances where metal tagging is utilized, metal ions can be read out by imaging mass spectrometry or multiplexed ion beam imaging (MIBI) or MIBI-TOF see, e.g. Keren, et al., Science Advances 9 Oct. 2019: Vol. 5, no. 10, eaax5851; DOI: 10.1126/sciadv.aax5851 (characterizing use of multiplexed ion beam imaging by time of flight instrumentation that uses bright ion sources and orthogonal time-of-flight mass spectrometry to image metal-tagged antibodies at subcellular resolution in clinical tissue section).

Use of CRISPR and other Nucleotide Modification Systems

In some embodiments, CRIPSR systems and other nucleotide modification systems are utilized for identification of the ‘z’ coordinate or location within the sample on the solid substrate. In certain embodiments, CRISPR systems and other nucleotide modification systems can be introduced to an identified x,y coordinate location on a solid substrate, or each location on the solid substrate. CRISPR and other nucleotide modification systems and/or their associated guide molecules can be included with the spatial barcode, including appended on the spatial barcode. In one exemplary embodiment, the CRISPR or other nucleotide modification system guide sequence is selected to edit the spatial barcode or adjacent of the spatial barcode, or otherwise map to the location on the solid substrate. In other embodiments, the CRISPR or other nucleotide modification system or other nucleotide modification system is designed as described herein to bind but not cleave at particular target molecules specific to a cellular subtype or upregulated to particular subtypes to provide a z axis indicator. In other embodiments, and as described herein the CRISPR or other nucleotide modification system systems can be utilized as CRISPR-mediated analog multi-event recording apparatus (CAMERA) systems, described herein, and used to record stimuli as interest and over multiple generations of cells. CRISPR and other nucleotide modification system systems can also be used to effect cell-signaling via, for example, cell-signaling pathways in the samples to identify or otherwise further evaluate the sample architecture and cell interactions. CRISPR and other nucleotide modification system can also be utilized for detection and diagnosis in diseases by aiding in the cell typing and subtyping and tissue profiling according to the methods disclosed herein.

The components of the CRISPR or other nucleotide modification system can be delivered by and suitable route or method. Such methods are generally known in the art and include delivery as coding polynucleotides, such as in the context of a vector or RNA, protein:polynucleotide complexes (RNPs) and others. See e.g., Lino et al., Drug Deliv. 2018 Nov.; 25(1):1234-1257. doi: 10.1080/10717544.2018.1474964; Wang et al., Cell. 2020 Apr. 2; 181(1):136-150. doi: 10.1016/j.cell.2020.03.023; Li et al., Biomaterials. 2018 July; 171:207-218; Kowalski et al., Mol Ther. 2019 Apr. 10; 27(4):710-728; Yip B. H., Biomolecules. 2020 May 30; 10(6):839; Zhang et al., Theranostics. 2021 Jan. 1; 11(2):614-64.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

### Class 1 Systems

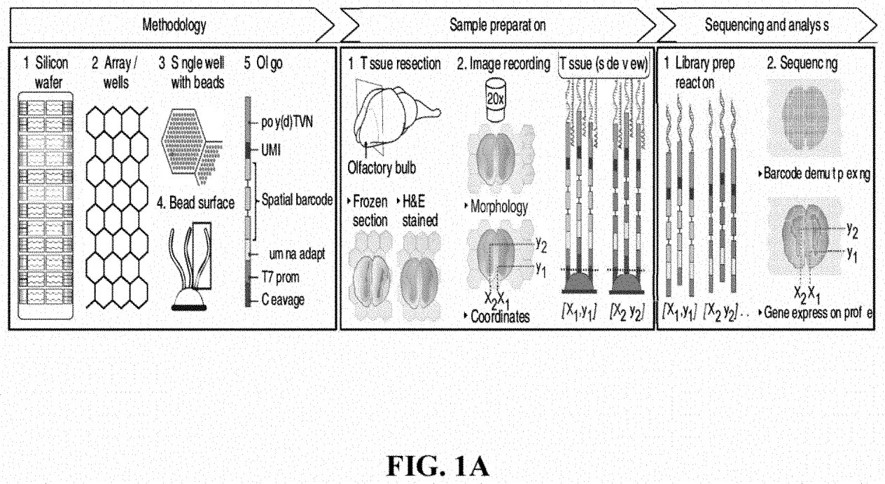

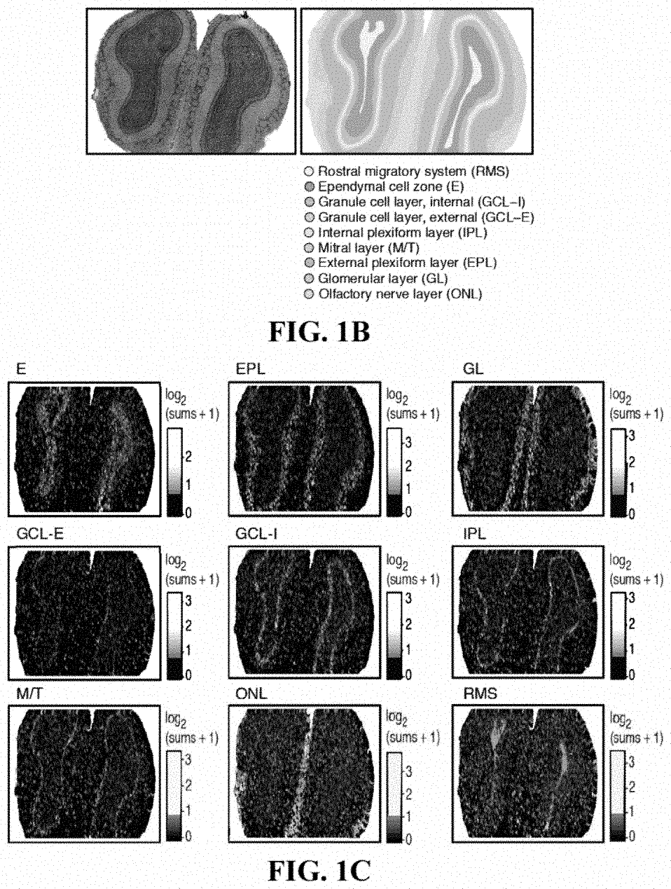

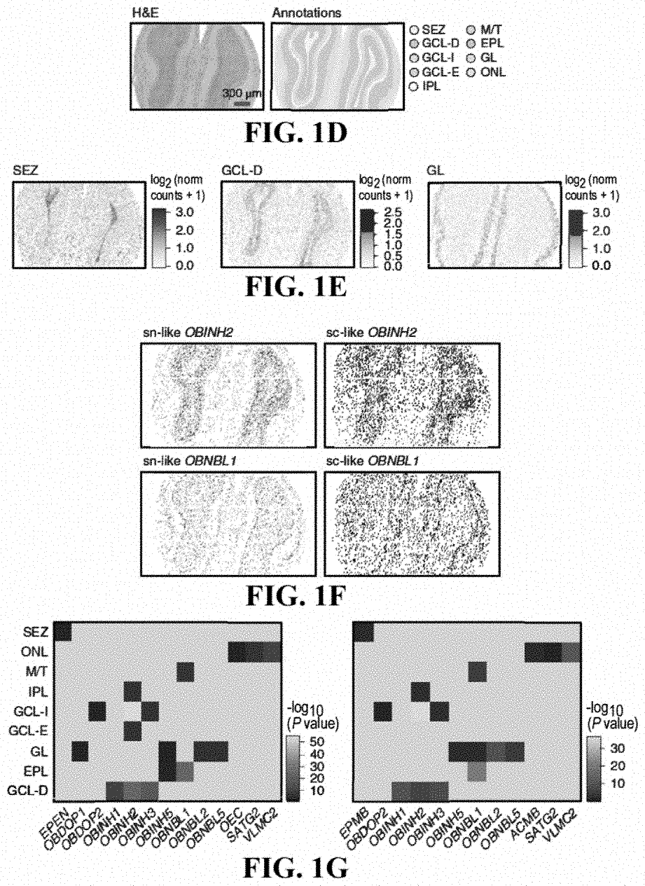

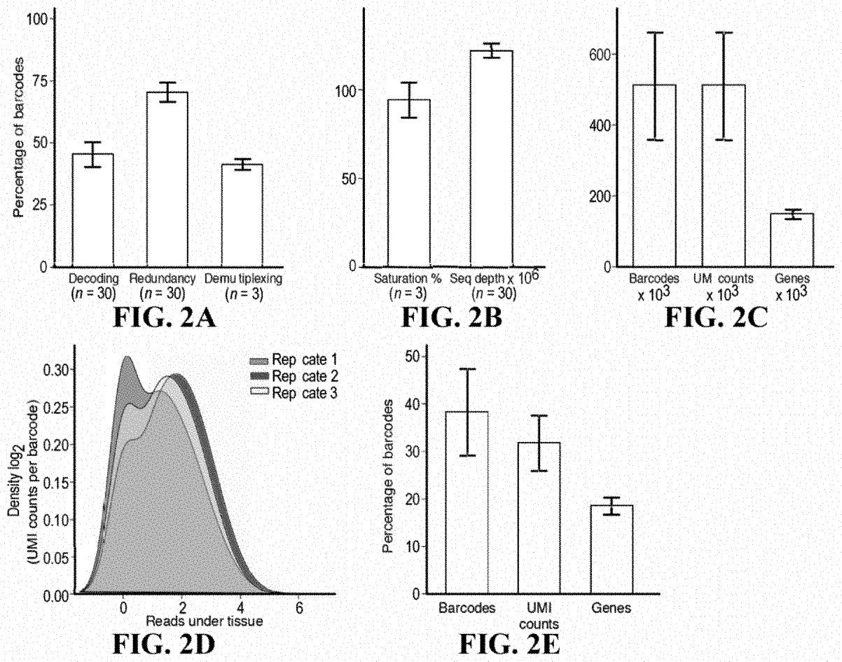

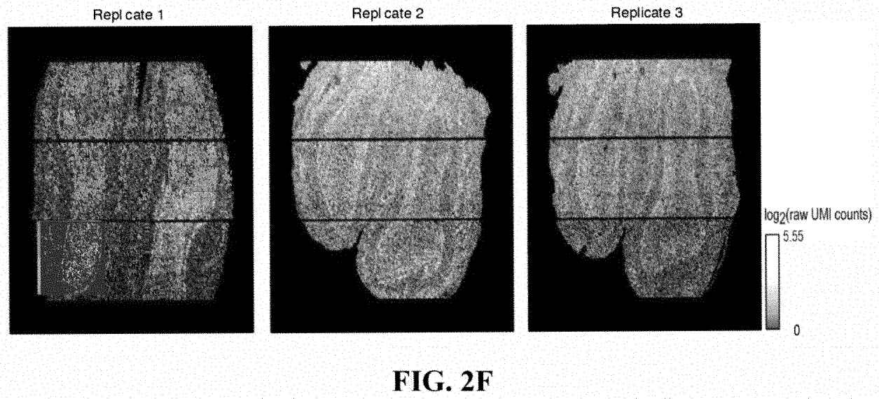

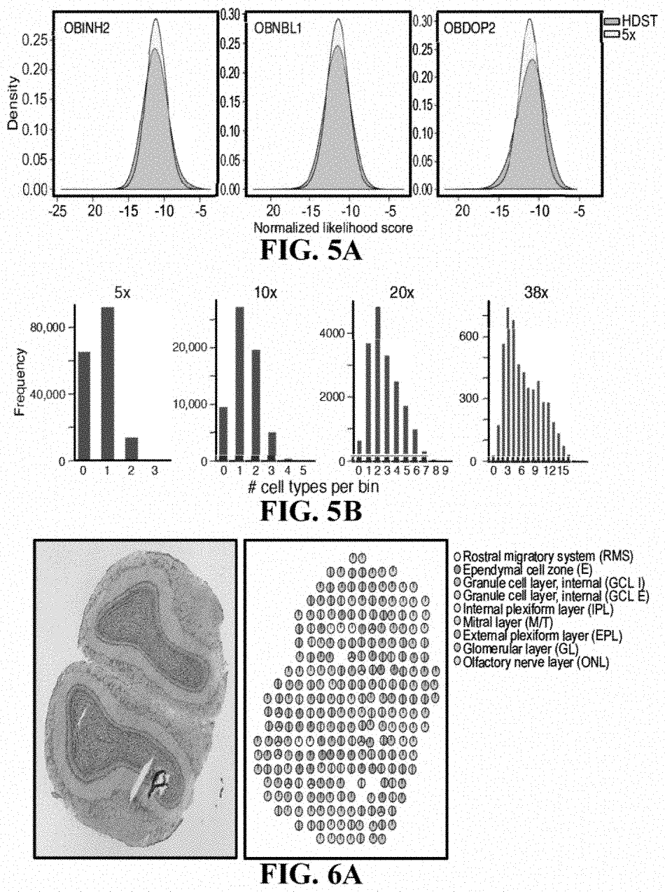

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020)., incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B 1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B 1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Nuclear Localization Sequences

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs). Suitable nuclear localization sequences are known in the art that can be incorporated with the present disclosure. Such sequences include without limitation, the NLS of the SV40 virus large T-antigen, having the amino acid sequence PKKKRKV (SEQ ID NO:1) or PKKKRKVEAS (SEQ ID NO:2); the NLS from nucleoplasmin (e.g., the nucleoplasmin bipartite NLS with the sequence KRPAATKKAGQAKKKK (SEQ ID NO:3)); the c-myc NLS having the amino acid sequence PAAKRVKLD (SEQ ID NO:4) or RQRRNELKRSP (SEQ ID NO:5); the hRNPA1 M9 NLS having the sequence NQSSNFGPMKGGNFGGRSSGPYGGGGQYFAKPRNQGGY (SEQ ID NO:6); the sequence RMRIZFKNKGKDTAELRRRRVEVSVELRKAKKDEQILKRRNV (SEQ ID NO:7) of the IBB domain from importin-alpha; the sequences VSRKRPRP (SEQ ID NO:8) and PPKKARED (SEQ ID NO:9) of the myoma T protein; the sequence PQPKKKPL (SEQ ID NO:10) of human p53; the sequence SALIKKKKKMAP (SEQ ID NO:11) of mouse c-abl IV; the sequences DRLRR (SEQ ID NO:12) and PKQKKRK (SEQ ID NO:13) of the influenza virus NS1; the sequence RKLKKKIKKL (SEQ ID NO:14) of the Hepatitis virus delta antigen; the sequence REKKKFLKRR (SEQ ID NO:15) of the mouse Mx1 protein; the sequence KRKGDEVDGVDEVAKKKSKK (SEQ ID NO:16) of the human poly(ADP-ribose) polymerase; and the sequence RKCLQAGMNLEARKTKK (SEQ ID NO:17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique, which are generally known in the art.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 1 (from Gleditzsch et al. 2019) below shows several Cas polypeptides and the PAM sequence they recognize.

TABLE 1
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCas13a) have a specific discrimination against G at the 3′ end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SETT/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No.WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the described elsewhere herein can be modified as appropriate using a prime editing system. See e.g., Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

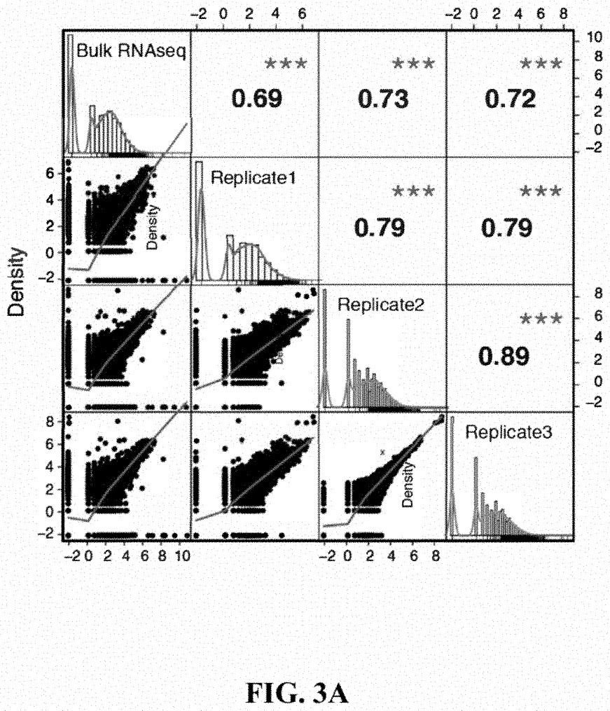

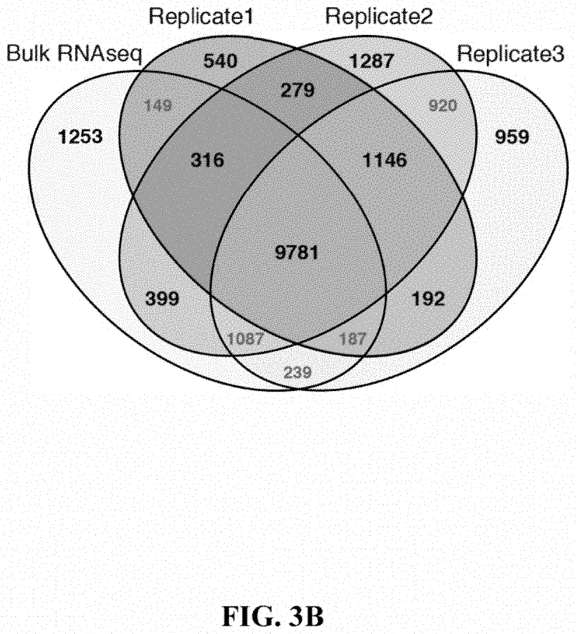

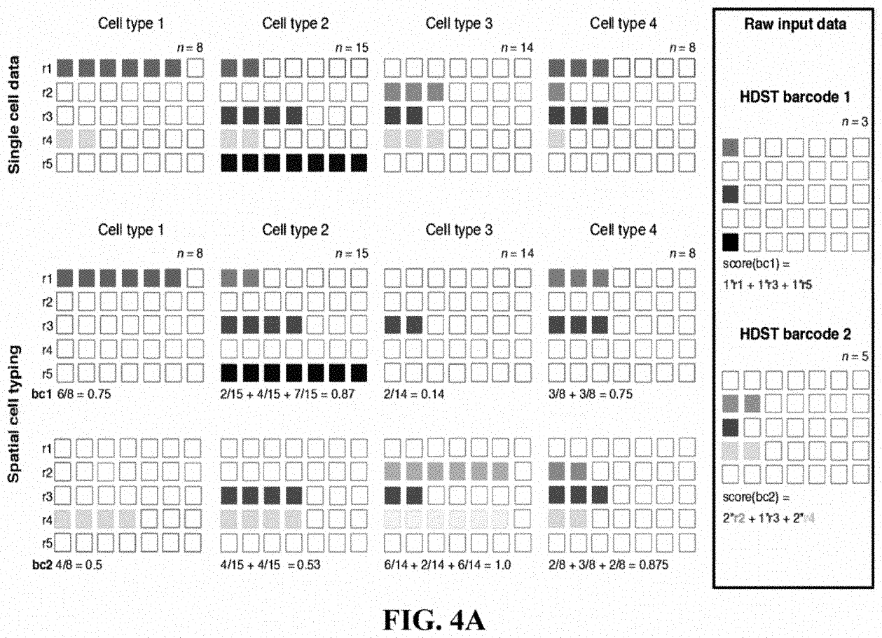

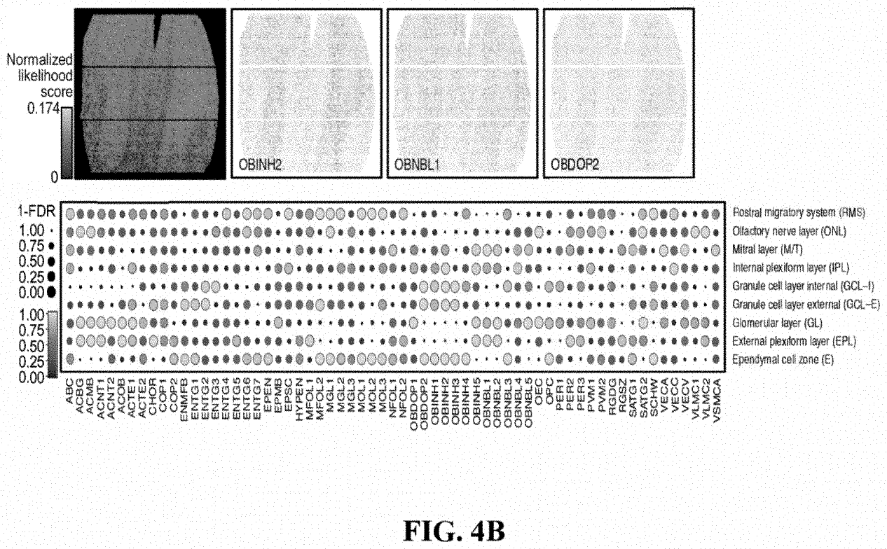

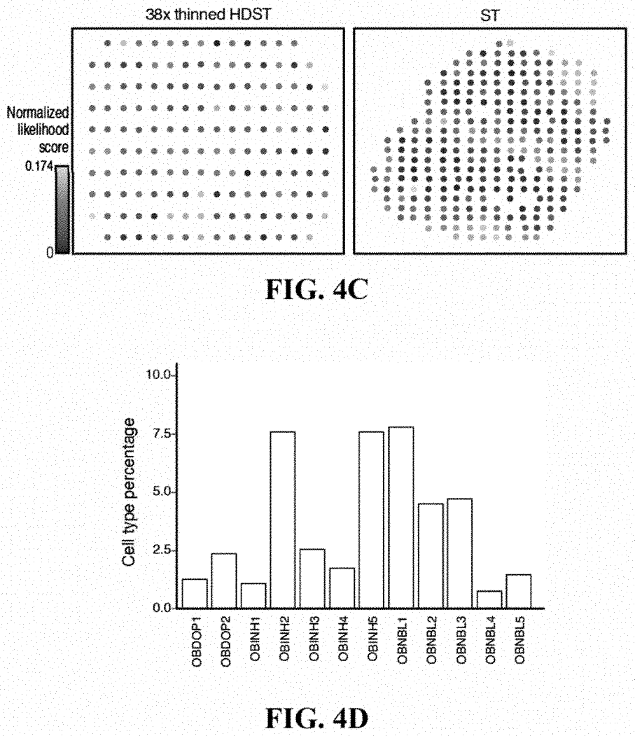

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide described elsewhere herein can be modified using a CRISPR Associated Transposase (“CAST”) system. In some embodiments a CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

Various aspects and embodiments of the invention may involve analyzing gene signatures, protein signature, and/or other genetic or epigenetic signature, in some instances based on single cell analyses (e.g., single cell RNA sequencing), or alternatively based on cell population analyses, as is defined herein elsewhere, and/or in combination with the sample morphology.

### Ablating a Single Cell Layer of Cells

In some embodiments one or more layer of cells are ablated. Ablating a single layer of cells can comprise contacting the solid substrate can comprise the selective destruction of a single layer of cells that have previously been processed and analyzed by the methods and systems as described herein so that a new layer of cells is exposed for processing and evaluation. In some embodiments, a laser ablation (Gahtan and Baier, 2004; Yang et al., 2004) or gene promoter for a toxin gene can be used to destroy a single layer of cells. In embodiments, enzyme-prodrug combinations can be used in specific cell populations. Although such approaches may be specific to specific cell populations, an advantage of this approach includes spatial and temporal control. (Curado et al., 2008; Davison et al., 2007; Montgomery et al., 2009; Pisharath et al., 2007; Zhao et al., 2009). In some embodiments, a cell-specific promoter can be used that expresses nitroreductase with subsequent exposure to metronidazole can allow targeted cell ablation. Irreversible electroporation (IRE), the irreversible permeabilization of the cell membrane through application of electrical pulses as described in Miller et al doi:10.1177/153303460500400615. Radiofrequency ablation or nanoparticles designed to absorb light for hyperthermic ablation may also be used. Approaches using light, heat, electrical pulses and/or combinations thereof can advantageously be tuned for the substrate and cells to be ablated.

### Devices, Systems and Kits

In certain aspects, the present disclosure provides devices, systems and kits for spatiotemporal analysis in-situ. The systems and kits may comprise one or more compositions and reagents described herein.

In some examples, the devices, systems, and/or kits may comprise sample permeabilization reagents, staining reagents, library preparation reagents, including for example, primers for reverse transcription, devices and/or reagents for performing spatial barcoding, devices and/or reagents for sequencing, amplification reagents, sequence reads analysis, or decoding, labels (such as dyes, optically active labels, and radiolabels), CRISPR or other nucleotide modification systems and/or components thereof, solid substrates optionally with pre-loaded spatial barcodes, capture molecules, or any combination thereof.

In some embodiments, the devices, systems, and/or kits can include one or more solid substrates, optionally pre-loaded with spatial barcodes, capture molecules, or any combination thereof and optionally configured on the substrate such that one or more fixed arrays are formed on the substrate. In some embodiments, the devices, systems, and/or kits can include conductive beads comprising a plurality of bead probes, optionally pre-loaded with spatial barcodes, capture molecules, or any combination thereof and optionally configured on the substrate such that one or more fixed arrays are formed on the substrate. In some embodiments, the solid substrate, optionally pre-loaded as previously described, is included in the devices, systems, and/or kits to form a chip (such as a lab on a chip) device. In some embodiments, the device or system is configured as a microfluidic device or system. In some embodiments, the device or system can include one or more chips, capillaries, microcapillaries, vessels, micro vessels, wells, microwells, or other discrete locations, which can optionally be pre-loaded spatial barcodes, capture molecules, or any combination thereof.

In certain example embodiments, the device is a microfluidic device that generates and/or merges different droplets (i.e., individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set.

In certain example embodiments, the system can include individual wells, such as microplate wells. The size of the microplate wells may be the size of standard 6, 24, 96, 384, 1536, 3456, or 9600 sized wells. In certain example embodiments, the elements of the systems described herein may be freeze dried and applied to the surface of the well prior to distribution and use. The solid substrate can be or be incorporated with the well(s).

The systems and/or devices disclosed herein may further comprise inlet and outlet ports, or openings, which in turn may be connected to valves, tubes, channels, chambers, and syringes and/or pumps for the introduction and extraction of fluids into and from the system and/or devices. The systems and/or devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In certain example embodiments, the systems and/or devices are connected to controllers with programmable valves that work together to move fluids through the device. In certain example embodiments, the systems and/or devices are connected to one or more controllers discussed in further detail below. The systems and/or devices may be connected to flow actuators, controllers, and sample loading devices by tubing that terminates in metal pins for insertion into inlet ports on the device.

The systems and/or devices disclosed herein may be configured as and/or also include elements of point of care (POC) devices known in the art for analyzing samples by other methods. See, for example St John and Price, “Existing and Emerging Technologies for Point-of-Care Testing” (Clin Biochem Rev. 2014 August; 35(3): 155-167).

In some embodiments, the systems and/or devices described herein can be used with a wireless lab-on-chip (LOC) diagnostic sensor system (see e.g., U.S. Pat. No. 9,470,699 “Diagnostic radio frequency identification sensors and applications thereof”). In certain embodiments, the present invention is performed in a LOC controlled by a wireless device (e.g., a cell phone, a personal digital assistant (PDA), a tablet) and results are reported to said device.

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

In preferred embodiments, the LOC may be a microfluidic device. The LOC may be a passive chip, wherein the chip is powered and controlled through a wireless device. In certain embodiments, the LOC includes a microfluidic channel for holding reagents and a channel for introducing a sample. In certain embodiments, a signal from the wireless device delivers power to the LOC and activates mixing of the sample and assay reagents.

In some embodiments, the device and/or system is configured to process, prepare, and/or position a sample in the device and/or system for analysis. In some embodiments, the device and/or system is configured to perform one or more reaction on one or more samples, including polynucleotide and/or polypeptide sequencing (described in further detail elsewhere herein), immunoassay, hybridization assay, or other sample analysis. In some embodiments, sequencing results in a detectable signal or other output that can optionally be detected by a sensor, which can be transmitted to a user interface (e.g., a computer, a wireless or portable device) or directly to a user via a direct visual signal (e.g., a binary signal, change in sample opacity, coulometric signal, and the like) to indicate the presence of a specific barcode, targeting moiety, or target in the vesicle. In some embodiments where a barcode is sequenced, the sequence can be detected (based on the sequencing method employed) and the results can be transmitted user interface (e.g., a computer, a wireless or portable device) or directly to a user via a direct visual signal (e.g., a binary signal, change in sample opacity, coulometric signal, and the like).

In certain embodiments, the device and/or system is configured as a handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader).

In certain embodiments, the device and/or system includes a light source and a detector for recording optical signals of any first and/or second analytical assay. The light source may be a laser, a photodiode array, or any similar light source. The detector may be a spectrophotometer, a photometer, a leumeter, a charge-coupled device, or any other similar device. The light source and detector may be appropriate for carrying out the optical imaging, optical diffraction, and optical-dependent second analytical assays or modalities, either for determining their number or other characterization or compositional analyses. For example, the light source and the detector would be configured as required by the optical imaging or optical diffraction methods and used to read optical signals generated at the sample surface or elsewhere within the device or system.

Microfluidic devices and/or systems disclosed herein can be or include silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides. In addition to reagents and devices, the kits may further include instructions for using the components of the kit to practice the methods. The instructions for practicing the subject methods may be generally recorded on a suitable recording medium. For example, the instructions may be printed on a substrate, such as paper or plastic, etc. As such, the instructions may be present in the kits as a package insert, in the labeling of the container of the kit or components thereof. In other embodiments, the instructions are present as an electronic storage data file present on a suitable computer readable storage medium, e.g., CD-ROM, diskette, etc. In certain embodiments, the instructions are not present in the kit, but means for obtaining the instructions from a remote source, e.g., via the internet, are provided. The kit may further include instructions for use as well as access to automated processing programs for the evaluation and processing of the kit, including imaging and processing of cell and tissue samples.

The invention is further described in the following examples, which do not limit the scope of the invention described in the claims.

### EXAMPLES

### Example 1—High Density Spatial Transcriptomics

Barcoded beads were produced with a split-and-pool approach. This resulted in sufficient barcode complexity to avoid large redundancies in duplicate spatial (x,y) locations. The array was made in a 1918×765 matrix for a total of 1,467,270 wells spread out into a hexagonal pattern. The well size was estimated at 2 μm with a 3 μm distance from center-to-center of each well. These decoded bead moieties in a patterned arrangement represented a high-density spatial transcriptomics (HDST) array. (FIG. 1A).

A tissue section was placed onto the bead array surface, stained and imaged. The tissue was gently permeabilized and the mRNA molecules captured onto the respective bead capture sequences, then effectively directly in-situ barcoded. This was followed by a reverse transcription reaction and library preparation.

With previously existing ST technology, ˜19% of the tissue area is spatially parsed into 100 μm features with a center-to-center distance of 200 Now, in HDST, given the barcode redundancy and decoding efficiency (FIG. 2A), as well as stringent barcode mapping cutoffs (FIG. 2A), Applicants randomly, effectively and spatially profile the same tissue area, now parsed into 2 μm pixels, while keeping the size of the profiled tissue section the same. The HDST bead array thus provides an increase of 2500 times in resolution compared to ST, with maximum packing density between two spatial measurements.

Given sequencing depth and library saturation (FIG. 2B), more than 68.3±5.9% (mean±standard deviation (sd)) of all reads generated in the library construction and more than 81.5±1.8% of all genes were located within the detected tissue boundaries (without using any lower cutoffs), with almost 140,000 barcodes generating spatially profiled data per assay (n=3) (FIG. 2C). Although an average number of filtered reads per barcode location was low (FIG. 2D), very limited background was detected outside the tissue boundaries, as compared to a very specific spatial in-situ tissue profile following the detected tissue boundary (FIG. 2E-H).

Next, Applicants compared average gene expression signatures from published total RNA-seq datasets from the mouse olfactory bulb to the averaged expression signatures obtained with HDST (FIG. 3A), with each of the replicates giving similar results, both to the bulk (r²=0.69±0.02; mean±sd) and to each other (r²=0.82±0.06; mean±sd). The majority of genes detected in the bulk data were also present in all HDST datasets (FIG. 3B). These results were consistent with previous studies (22) and Applicants proceeded to explore the data further.

### Single Cell Typing in HDST

First, Applicants wanted to pair the HDST spatial data to both cell type information and morphological information by imputing publicly available and annotated cell type signatures from two published datasets (25, 26). Cell types were assigned to the spatially barcoded signatures using a combinatorial approach. Briefly, the observed gene count distributions per cell type were assumed using the single cell RNA-seq datasets and then likelihoods for all spatially barcoded genes belonging to each of the detected cell types calculated (FIG. 4A). Applicants observed that 97.4% of all spatially barcoded gene expression profiles could be connected to one cell type with a cell type likelihood score. Then, specific spatial cell type patterns could be observed and compared to morphological data annotated from the H&E stain (FIG. 1B), and cell type enrichment scores calculated for each individual cell layer (FIG. 4B) with some populations exhibiting layer-specific patterns (FIG. 4B). Applicants also downsampled and thinned HDST data in a stepwise manner, with the lowest resolution now mimicking ST data, and used one ST dataset in the cell type assignments (22) (FIG. 4C). As expected, ST cell type scores were lower given convolved cell signatures per spatial measurement in cell-dense areas while higher is cell sparse and layer-specific areas.

Applicants performed spatial cell typing of all regions sampled in a tissue section over interconnected anatomical regions. At at fine-grained level, Applicants explored connectivity between different populations in the main olfactory bulb. With high likelihood scores, Applicants confirmed that a few different neuronal, oligodendrocyte (MOL and myelin-forming mature oligodendrocytes; MFOL), astrocyte (AC and OEC), immune (MGL) and vascular (VLMC, SAT and SCHW) populations were present in the analyzed section, including both GABAergic neuronal populations (OBINH) and dopaminergic neurons (OBDOP), neuroblasts (OBNLB) and olfactory-bulb enriched astrocytes (OEC and ACOB). That the largest class (20.55%) of detected olfactory neurons were GABAeric (FIG. 4D) is in line with previous results (26, 27). Also, individual neuroblast populations were identified in the mitral and external plexiform layers (OBNLB1 and OBNLB2) presenting more differentiated cells and some the ependymal zone (E) and rostral migratory system (RMS) cells (OBNLB3) presenting potentially non-terminally differentiated neuroblasts. These neuroblasts have previously been reported associated with specific layers (26).

### Neighborhood Differential Spatial Analysis Between Morphological Layers

Given the spatial sparsity, increased subcellular resolution and data distribution, Applicants divided the analyzed area into bins and summed the spatial gene expression profiles over the neighboring (x,y) measurements within each bin. This gave on average 3.5±1.9 (mean±sd) (x,y) bead observations with 10.7±9.1 (mean±sd) read counts per bin and resulted in very limited convolution of the spatial transcriptomic data (FIG. 5A-B). This represented binned spatial gene expression.

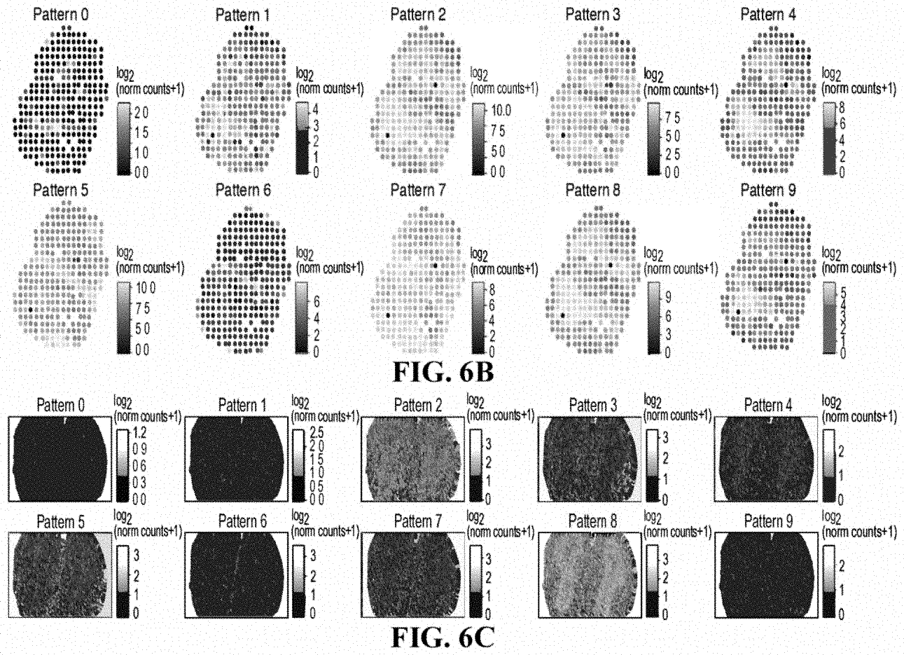

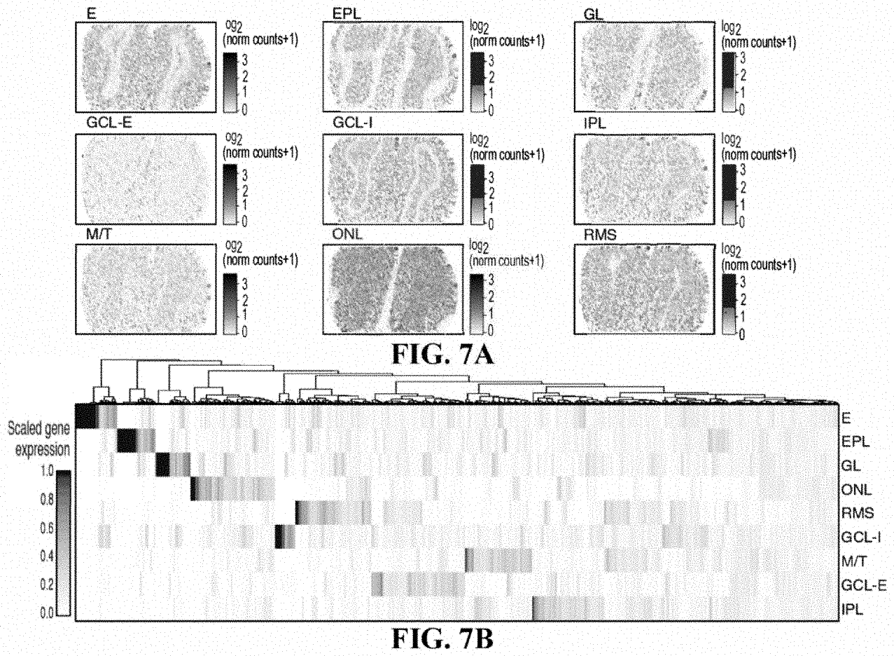

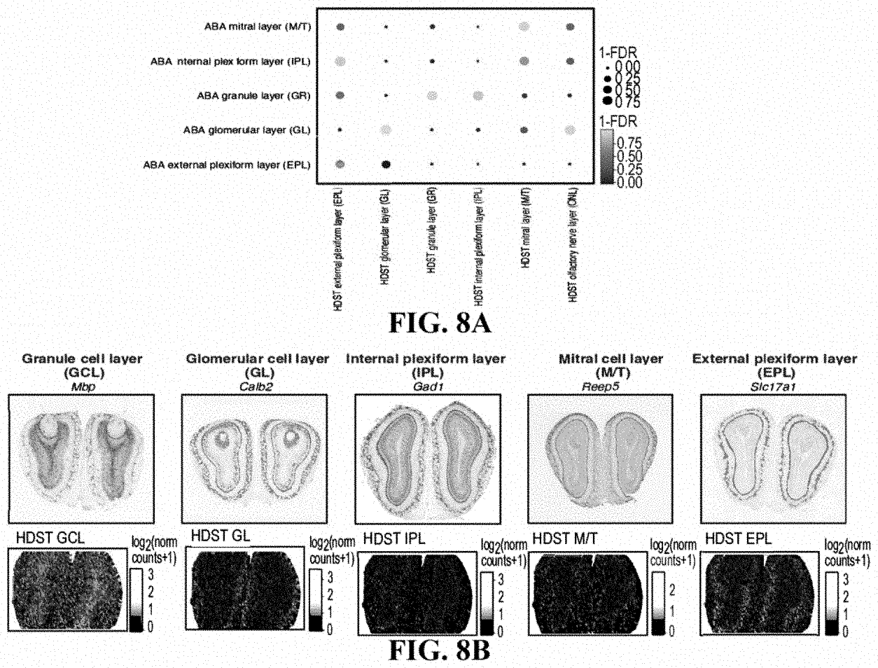

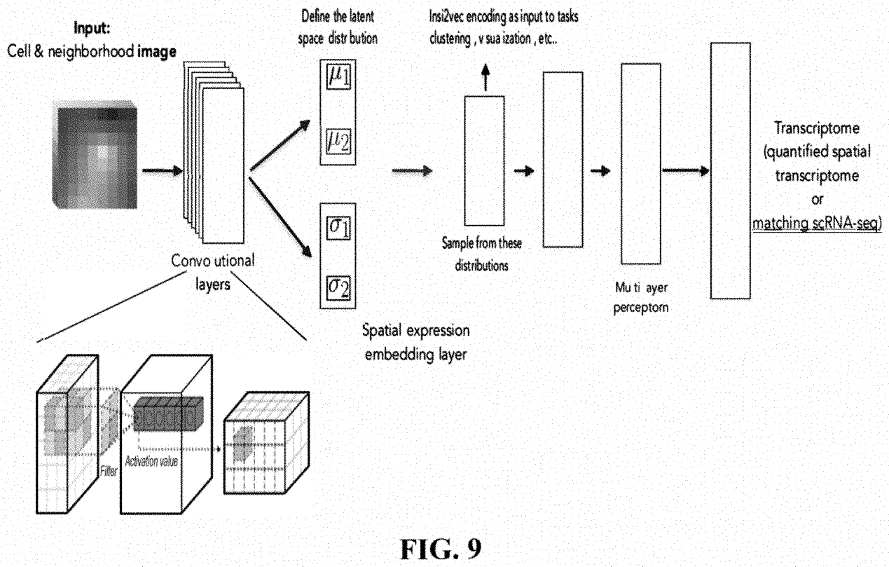

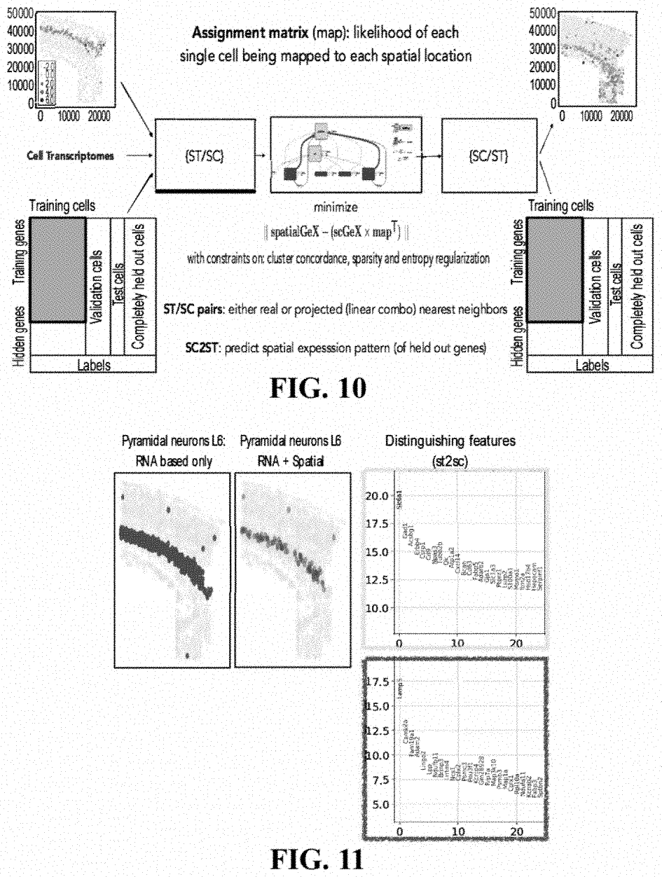

Automatic spatially variable patterns (28) coupled to convolved morphological areas (FIG. 6A) could be detected in the standard ST approach (FIG. 6B). These gene coexpression signatures could also be reconstituted in HDST data (FIG. 6C). Some of the convolved signatures matched well to greater morphological area while others, as expected, ended up making unspecific gene expression patterns in HDST. Given the great increase in resolution in HDST, Applicants next explored whether one could robustly detect differentially expressed (DE) genes between the different fine morphological layers in a supervised manner. For the binned spatial data, Applicants used a smoothing Gaussian filter, which led to 16.9±11.3 (mean±sd) reads per bin, and then performed a two-sided t-test (FDR<0.1), which resulted in DE signatures specific to morphological layers (FIG. 1C, FIG. 7A-B). Layer-enriched upregulated DE genes (LFC>1.5) that were also detected in the Allen Brain Atlas (ABA) (9) coronal dataset were assigned to the correct ABA layer information (FIG. 8A) and the top genes found in both datasets exhibited very specific layer-based patterns (FIG. 8B).

### Discussion

Molecular states interact based on both their nearby and distant stimuli, making a spatial communication network. Spatially resolved transcriptomics provides a tool to reveal biological insights into these molecular states and neuroanatomical, temporal and morphological structures by providing transcriptomic signatures that are the consequence of complex cellular circuitry coupled to spatial information that is critical for interpreting function.

High-density spatial transcriptomics is a robust high-resolution approach providing in-situ spatial information on cell dynamics. The technology relies on standardized tissue, molecular, bead-array and imaging tasks, making it a resource deployable by the broader scientific community with focused on new biomedical discoveries. HDST uses standard histological stains, providing the means to correlate morphology to gene expression as well as a framework to correlate cell type and state information to the extracellular environment. High-density spatial transcriptomics and its further development will aid the increased understanding of cell type and spatially resolved classifications and connections.

### Materials and Methods

### Array Design

A split-and-pool approach was used to generate a total of 1,079,642 different bead entities. A primer precursor was linked to the bead surface with a cleavable d(U) linker. After linkage, in order to increase the bead pool size (determined as number of unique beads in the pool) a ligation approach was used. Briefly, 3 sequential ligation steps were performed adding 15 bp, 15 bp and 14 bp of barcode sequences using a bridge oligonucleotide, enabling double-stranded ligation using T4 DNA ligase with the ligation oligonucleotide added in a ratio of 2:1 to the precursor oligo sequence. In the following ligation step, the newly ligated sequence acted as the precursor. In the final ligation step, the last barcode sequence was followed by a 6 bp unique molecular identifier and a stretch of 20 (d)Ts and VN to ensure efficient mRNA capture on the surface. The complete bead pool was used to load a total of 1,467,270 predefined well positions covering a 13.7 mm² area (5.7 mm×2.4 mm). A total of 24 such areas were made on each slide.

### Samples

Adult C57BL/6J mice were euthanized and their mouse olfactory bulb dissected. The samples were then frozen in an isopentane (Sigma-Aldrich) bath kept at −40° C. The samples were then transferred to −80° C. The frozen bulbs were embedded at −20° C. in Tissue-Tek OCT (Sakura) compound. Cryosections were taken at 10 μm thickness and deposited on prechilled slides containing barcoded arrays.

### Tissue Staining and Imaging

Tissue sections were first adhered to the surface by keeping the slide at 37° C. for 1 min. Immediately after, a fixation step on the slide surface was performed using 4% neutral buffered formaldehyde (Sigma-Aldrich) in 1× phosphate buffered saline (PBS, pH 7.4) for 10 min at room temperature (RT). The slides were then washed once in PBS to ensure proper formaldehyde removal. The sections were stained using standard hematoxylin and eosin staining described in Stahl et al (22). Imaging system used was a Ti-7 Nikon Eclipse. In short, a NB filter was used in fluorescent mode to expose the samples to a bright field light source and the reflections collected on a color camera. This enabled histological imaging of a dark slide on a standard epifluorescence microscope.

### Library Preparation and Sequencing

The following steps were described in detail in Stahl et al (22). In short, tissue sections were gently permeabilized using exonuclease I buffer (NEB) and pepsin. This was followed by in-situ cDNA synthesis overnight at 42° C. using Superscript III (Thermofisher) supplemented with RnaseOUT (Thermofisher). This ensured that the transcript information was transcribed and spatially barcoded into cDNA molecules.

Tissue sections could then be digested using proteinase K (Qiagen) and the barcode information cleaved using a Uracil-Specific Excision Reagent (NEB) targeting the 5d(U) stretch at the 5′ end on the barcoded oligonucleotides. The collected material was then processed as according to Jemt et al (29). The finished libraries were sequenced 2×150 bp on a Illumina Nextseq 500 instrument with v2 chemistry.

### ST Pipeline Processing

The fastq files were processed using the ST Pipeline v1.5.1 (30). The forward read contained both the barcode sequencing and the bridge sequence used for the sequential ligation steps. The bridge sequences were trimmed and removed prior to any barcode mapping steps. The transcripts were mapped with STAR to the GRCm38 (v8) reference. The annotated reads were counted using the HTseq count tool and then the UMI duplicated sequences collapsed using a hierarchical clustering approach and paired to spatial barcodes demultiplexed using TagGD (31) (kmer 11, mismatches 4, hamming distance method for barcode collapsing). This generated a counts matrix with a Cartesian (x,y) coordinate assigned with gene expression information.

### ST Image Processing

In order to match the histological image and the counts matrix generated with the ST Pipeline, Applicant needed to assign image pixel coordinates to the centroids of each bead well. This ensured proper alignment tissue boundaries in the image and could select the barcodes located spatially underneath the tissue boundaries. The same approach was taken to detect the arrays' boundaries and corners, upon which a perfect well matrix can be assumed given standardized production and quality control specifications for each slide (32). Pixel coordinates can now easily be the translated into fixed centroid (x,y) coordinates using the total detected area of the array. The coordinate names then matched the decoder file used in the ST Pipeline processing step.

### ST Image Annotation

Images used in the study were annotated using a user interface enabling interactive selection of spatial barcodes and their (x,y) coordinates based on the tissue morphology into 9 distinct regions present in the mouse olfactory bulb i.e. Olfactory Nerve Layer (ONL), Granular Cell Layer External (GCL-E), Granular Cell Layer Internal (GCL-I), Ependymal Later (E), External Plexiform Layer (EPL), Mitral Layer (M/T), Internal Plexiform Layer (IPL), Rostral Migratory System (RMS) and the Granular Cell Layer (GL). The same tool was used to annotate regions in tissue sections produced in the Stahl et al (22) study. In the ST case, more than one tag was assigned per (x,y) spatial spot location in case the spot area spanned more than one layer. The annotation tags could then be exported and used in further analyses.

### Data Processing

Raw decoded spatial arrays and corresponding decoder files were shared by Illumina after bead arrays production in the standard format. Barcode decoding (including empty wells) and redundancy percentages based on the Illumina decoding process were calculated. Public total RNA-seq datasets were downloaded from NCBI's SRA project with accession PRJNA316587. The data was mapped to the mm10 reference and UMI filtered using the ST pipeline v1.3.1. Averaged and naively adjusted gene expression signatures (28) corresponding to the “Bulk MOB” data from Stahl et al (22) were compared to those of the three replicates created with the high density approach and normalized the same way. Allen Brain Atlas (ABA) gene lists were downloaded from the API using the ConnectedServices module of the allensdk Python package version 0.16.0. The differential search was performed within the MOB annotation only in a one-layer-vs-all and coronal-only data. The ST data as a counts matrix was downloaded from http://www.spatialtranscriptomicsresearch.org/datasets/doi-10-1126science-aaf2403/.

### Single Cell Typing in HDST

Applicants downloaded the pre-processed normalized matrices per cell type from Zeisel et al (26). Specific gene co-expression signatures resulting from their analyses were used as sanity checks in the spatial data processing, as the authors suggested a region each cell type corresponded to. For each of the identified and annotated cell types, the probabilities to capture each of the genes were calculated as gene-wise relative frequencies. For each cell type, provided mean gene-wise expression values were divided by the sum of all mean gene-wise expression values for this cell type, such that per cell type the gene-wise relative frequencies across all genes summed up to 1. To assign the most likely cell type to each spatially barcoded transcriptome, likelihood—scores were calculated for each of the potential cell types by summing the previously calculated cell type specific gene-wise relative frequencies and weighted by the counts for each of the genes captured by the respective spatially barcoded transcriptome.

Finally, the cell type with the highest likelihood score was assigned to the respective (x,y) position. Normalized likelihood scores were calculated for each barcode by dividing the assigned maximum likelihood score by the sum of its weights.

### Binning of Spatial Data

The total area of each HDST array area was divided into bins each covering an area of XxX beads (X={5,10,20,38}), and summed the spatial gene expression profiles within each bin. In order to ensure appropriate bin sizes, Applicants first considered all manufactured wells as a 1918×765 matrix. On average, around 1370 (x,y) wells filled with beads would size up to one ST spot (100 μm; x=38) when taking into account the center-to-center distance between two wells. From there, 4 additional bin sizes were calculated.

First, it was needed to thin the binned data containing 1370 wells per bin and take every second bin into account in both x and y directions. This was to ensure space between two ST spots would be accounted for. Applicants did not take into consideration that this bin actually represents 63% of the transcriptome profiled per ST spot due to the well packing density as space between two wells. Then, Applicants proceeded to make bins with fewer wells per bin in a logarithmic manner until reaching the smallest bin with an average of 3.5±1.9 (mean±sd) wells with beads containing transcriptome information. To access data convoluted as a result of binning, the frequencies of individual cell types detected per bin were calculated as compared to non-binned data in cases where more than one bead was present per bin.

### Spatial Differential Expression Analysis

Automatic and spatially variable gene patterns were detected in the ST dataset using SpatialDE (28). The number of expected variable regions was set to n+1 where n represented the number of unique morphological regions annotated in the dataset. A minimal number of 3 variable and co-expressed genes was set to ensure no overclustering was performed on the data. The highest ranked scores for each pattern were compared to the (x,y) coordinates assigned to morphological regions annotated based on manual image analysis as described above.

Binned HDST data was smoothed using a Gaussian kernel with 0.5 standard deviations equally in both x and y directions. The smoothed binned data was then scaled such that the maximum expression value stayed the same. Applicants performed a two-sided t-test (FDR<0.1) to identify DE genes for each HDST morphological region. The maximum top 500 genes identified per morphological layer with a log 2 fold change (LFC) of 1.5 (one vs rest) were identified as differentially expressed and used in further analyses. Smoothed HDST data was normalized to an average UMI count per bin. The spatialDE automatically assigned gene coexpression patterns were plotted in the normalized HDST data for comparison to patterns assigned from HDST data alone.

### Validation of Differentially Expressed Genes

To validate layer specific genes identified through differential expression analysis in the HDST data, enrichment analysis was performed using layer specific gene sets from the Allen Brain Atlas as reference. Genes with a layer specific LFC of greater than 1.5 (implying upregulation) and FDR<10% as per differential expression analysis in the HDST data were tested for enrichments in the layer-specific gene sets (“expression fold” change greater than 1.5) from the Allen Brain Atlas. Only genes passing the respective fold-change thresholds in both data-sets (n=221) were included in the analysis. The significance of enrichments was determined using a one-sided Fisher's Exact Test. Images for the top gene present in each layer were downloaded from ABA's High Resolution Image Viewer and stitched using Fiji (33).

### Data Availability

The data has been deposited to NCBI's GEO archive.

### Example 2—Defining a Cell by Intrinsic and Extrinsic Features

Insi2vec is a new generative variational autoencoder used to define cell subsets by combination of intrinsic and spatial features. Application to neural tissue described here: generalizes across patients, recovers new states and can be used with any spatial method at cellular resolution, including proteins, antibodies RNA.

1) Step 1: use v2 to project single cell data onto the spatial data.

Input: (i) sc matrix (sc_cells×sc_genes) and (ii) st matrix (sc_cells×sc_genes)

Output: Projected ST matrix (st_cells×sc_genes) (using v2)

Consistency checks included Clustering original st_matrix and then clustering the projected st_matrix; then computing the clustering NMI between the two results. They are quite concordant. For the st genes, there is >0.95 correlation between projected and real gene expression.)

(for common genes:correlation with just v1 is about 0.6 (pretty high for osmFISH considering the sc data was a completely different project of the same region), correlation with v2 is 0.95+) (obtained similar values of correlation for melanoma for v1/v2 as merfish

Use the projected ST matrix to update the input image. Input image now has a #sc_genes (>500 genes) as the #channels, instead of #st genes as the #channels(<50)

2) Step 2: use variational/deep generative insi2vec on the [x_dim, y_dim, sc_genes] dimensional image to predict the original st_matrix (st_cells×st genes) as labels to learn the insi2vec embedding and do clustering, etc to discover novel sub populations. (note, Applicants could also use the projected_st_matrix (st_cells×sc_genes) as the labels here if Applicants want; st_matrix (st_cells×st genes) as the labels because they are true ground truth labels as measured by experiment and not model predictions of v2 (the model predictions of v2 are being incorporated in the input in the form of the expanded image and that's how used to inform the embedding)

3) Step 3: Use differential expression tests on the projected_st_matrix (st_cell×sc_genes) to identify the markers/features for the spatio-transcriptomic subpopulations identified by the end2end sc2st variational/generative insi2vec (referred to as insi2vec herein). Results are shown in FIG. 11, with independent validation from Tasic et al, Nature Neuroscience volume 19, pages 335-346 (2016).

Insi2vec allows 1) Inferring from multiple orthogonal modes of measurement captured by in situ methods to combine cell-intrinsic and cell-extrinsic features in our definition of celltypes and (2) Predicting spatial expression patterns of genes. This has allowed Applicants to directly operate on the images and are genuinely spatially-aware (as opposed to just treating cells in in-situ datasets simply as another type of scRNAseq dataset).

Use of osmFISH Data from Somatosensory Cortex to Illustrate Method

Step 1) Input: (a) IMAGES from an in-situ transcriptomic experiment/(b) A corresponding quantified version of the image: A spatial gene expression matrix which is of the form ([x_coordinate, y_coordinate, z_coordinate, gene_1, gene_2, gene_3, gene_4, . . . gene_k]).

Operating directly on the image data allows natural integration of spatial gene expression patterns of surrounding cells and global gene expression patterns like gradients (which are quite important, especially in the context of the brain).

Although ideally one would want both (a) and (b), but since there are often published datasets where raw image data is a bit tricky to acquire (for e.g., :merFISH); in the development of this method, the assumption is only access to (b) from Step 1; and with only (b), one can reconstruct the actual image (a) using radial basis function interpolation (to recapitulate important global spatial gene expression patterns like gradients, often lost in quantification and not really captured by a myopic view of spatial tx matrices simply as scRNAseq matrices in ‘multi modal integration’ approaches).

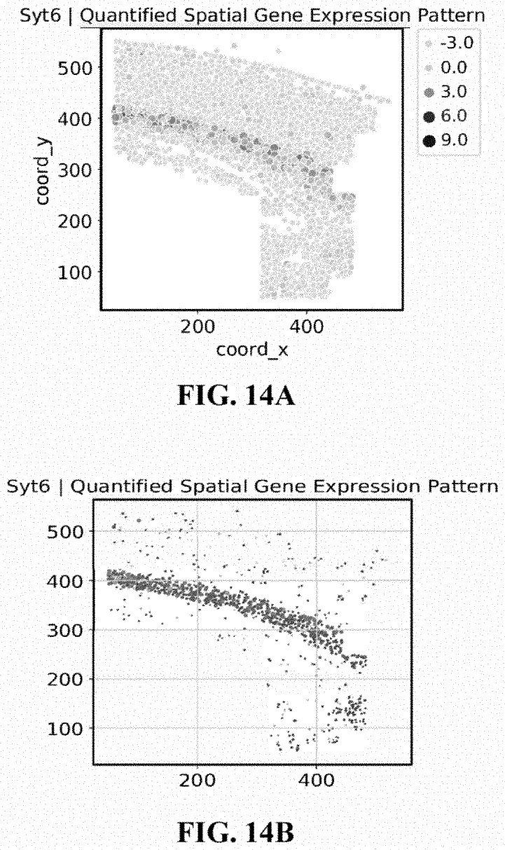

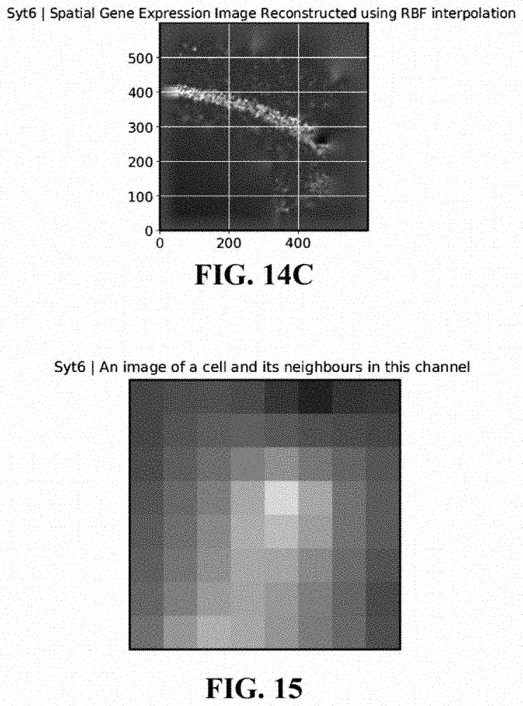

osmFISH data from the somato-sensory cortex is used illustrate each step. FIG. 14A provides an example spatial gex matrix (1b) from osmFISH for a gene (Syt6) and FIG. 14B provides a scatterplot. FIG. 14C is the result of reconstructing the image (to get (a) using a linear radial basis function interpolation. In summary, at the end of step 1, there are both (a) IMAGES from the in-situ transcriptomic experiment and (b) A quantified spatial gene expression matrix corresponding to that image. Additionally, having access to (c—A matching scRNAseq dataset from the corresponding tissue) allows prediction gene expression patterns of new genes (and all other tasks addressed by v1 and v2), discussed further infra.

Step 2) Armed with (a—Image) and (b—quantified spatial gene expression matrix), the spatio-transcriptomic integration of cell intrinsic (e.g., :transcriptomic) and cell extrinsic (e.g., :neighbouring cells, global gene expression gradients, neighbourhoods) features for a novel way of defining cell types is conducted.

Previous approaches that did not work include representing each tissue as a graph where each cell would be a node and an edge between cells that were neighbors to apply generalized Encoder-Decoder architectures on these graphs for learning a single vector for each cell that combines spatial and transcriptomic information. Multiple attempts on various datasets were made towards using graph encoding frameworks (most notably using GraphSAGE and FastGCN). Briefly, these attempts did not yield satisfactory results. Although seemingly elegant, the graph representation itself had issues. Without being bound by theory, representing the tissues as a graph where nodes are cells and there are edges between neighbors doesn't adequately capture the global effects of the spatial gene expression patterns and gradients because graph embedding approaches often rely on random walks in the local neighborhood surrounding a node. There isn't a natural way of adequately representing the effects of continuous nature of local and global structure in embedding methods for individual nodes in graphs satisfactorily for the desired applications

Instead, Applicants chose to operate directly on the in-situ method image (1a) (either directly from the data/reconstructed using the rbf interpolation from (1b)) of the location of the cell and a region around it e.g., :+−4 pixels in all directions). Operating directly on the image has many advantages, including the fact that Applicants no longer artificially discretize the neighborhood and allow for spatial gene expression patterns like gradients (local gradients—because they are directly evident in the image itself; and global gradients because of the rbf interpolation on the whole tissue allows propagation of gradients by interpolation where data is missing) to be incorporated into our definition of cell types.

Now given an image of the cell and its neighbors, Applicants used state-of-the-art convolutional autoencoder architectures from vision literature. These have significant advantages over (i) fully connected autoencoder architectures like scVI, scGen, etc:including the fact that convolutional operations make the network spatially aware and (ii) Graph embedding methods (including graph convolution methods) because of the issues with the graph abstraction described above.

An example image of a cell and its neighbor in FIG. 15 is a single channel corresponding to the Syt6 gene—the same one from the plot of FIG. 14C, Notice the gradient and also note that the full image has 33 channels.

Then, for the convolutional autoencoder:

>Input: a 33 dimensional image of a cell in its neighborhood is the input for each cell.

->Output (label): (i) The quantified transcriptome of the cell/(ii) A scRNAseq transcriptome of a matching single cell (using v1/v2).

->NN architecture: A convolutional autoencoder (The design choices made for the architecture used here based on experience and a heuristic described below, but other vision analog CNN AE architectures should work here)

For intuition, one can think of this approach as directly trying to learn a model of how the gene expression patterns of the cell and its neighborhood influences the cell, and allows Applicants to build on and advance interpretability frameworks for understanding tissue behaviour for future applications)

Step 3) Use the spatio-transcriptomic embedding for tasks like clustering/visualization/any other operation that Applicants can define on vectors. The spatio-transcriptomic embedding now incorporates information of the cell and its neighbourhood and thus more and richer information that allows discovery of new biology and substructure.

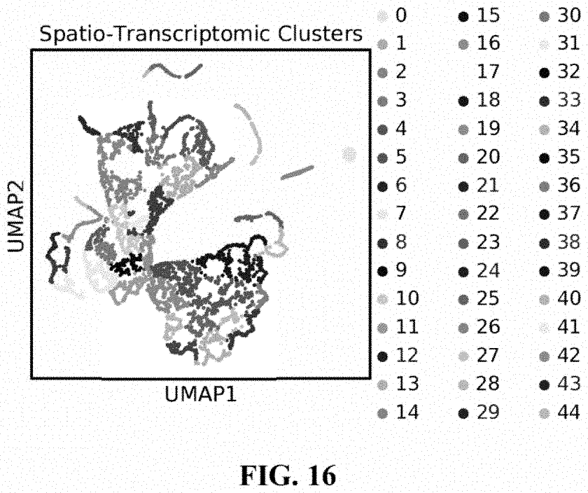

Continuing with the osmFISH illustrative example, now one can use the learned spatio-transcriptomic embeddings to discover new spatially relevant subsets of cells using traditional clustering approaches on this learned vector (FIG. 16).

Reproducing analysis previous osmFISH data, and identifying the pyramidal neuronal population using just the transcriptomic information from osmFISH in accordance with the published result in the original paper, Applicants utilized the umap coordinates from the image through the spatio-transcriptomic embedding of FIG. 16 and replaced the labels with the labels from the osmFISH known data, based solely on the transcriptome and without the spatial information, the layer nomenclature (pyramidal 11,12 etc) is using the spatial locations.

Spatio-transcriptomic embeddings uncover new spatially defined sub-populations of pyramidal L4 neurons and spatio-transcriptomic embeddings suggests heterogeneity and potentially interacting sub-populations of pyramidal L6 neurons. Using the clustering from the learned spatio-transcriptomic embeddings, Applicants observed two spatially associated cell subsets. (FIG. 11 middle panel of Pyramidal Neurons L6 with RNA plus Spatial overlay).

The analysis can then extend this spatio-transcriptomic framework to the spatial gene expression and other predictions tasks. Of note, osmFISH only had 33 genes and the top marker for the yellow subpopulation (0) wasn't in the osmFISH data but was predicted using insi2vec (Slc6a1) FIG. 11. These are known subtypes in layer 6 also indicated in Tasic et al and Zeisel et al 2018, with validation from Zeisel et al 2018: (http://mousebrain.org/genes/Lamp5.html, http://mousebrain.org/genes/Slc6a1.html). And the subtypes from layer6 that express these markers allowed Applicants to have the exact location rather than previous work guessing at layer estimates. http://mousebrain.org/celltypes/SCINH1.html (potential candidate for sub class 0), http://mousebrain.org/celltypes/TEGLU3.html (potential candidate for sub class 1).

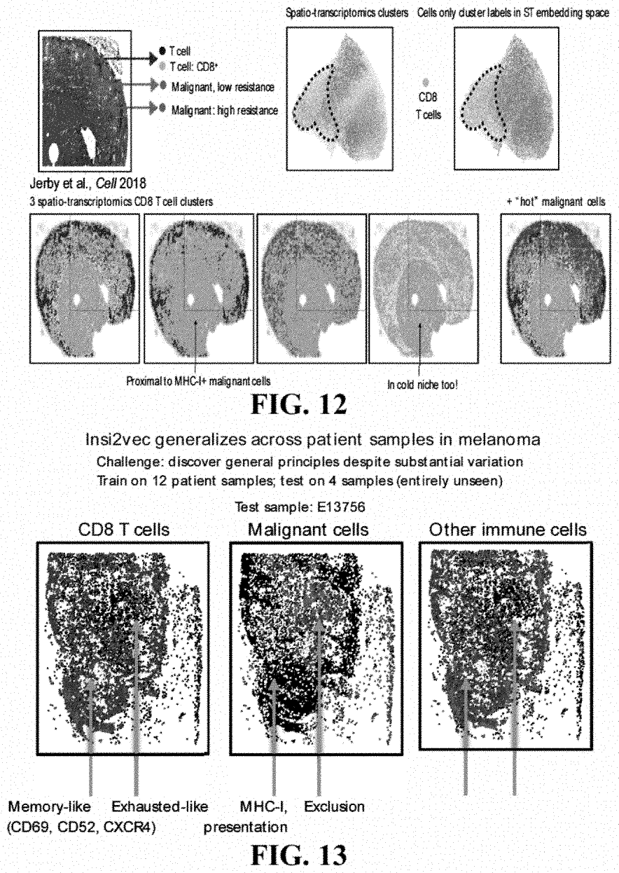

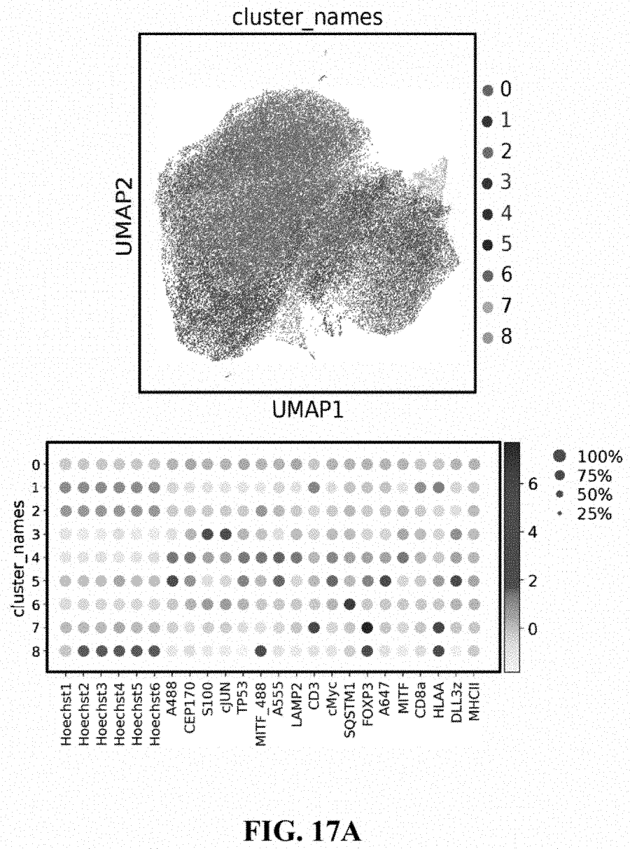

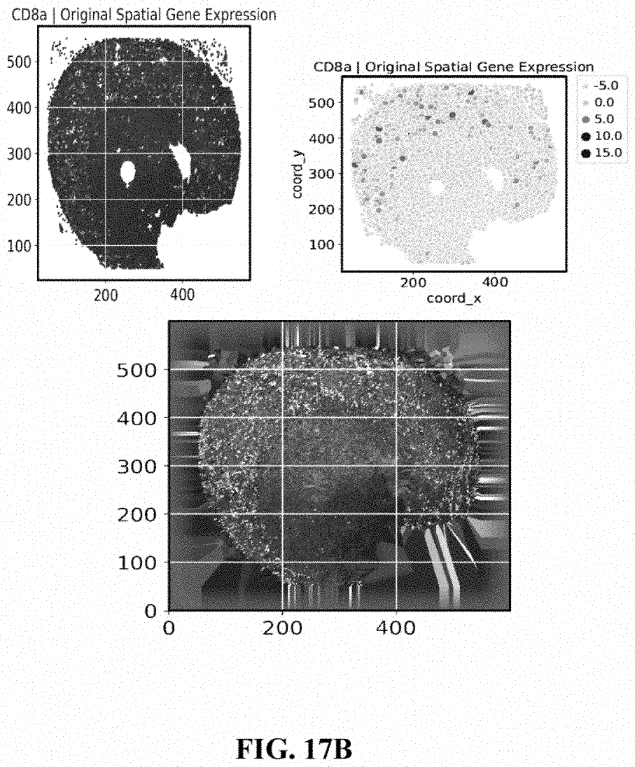

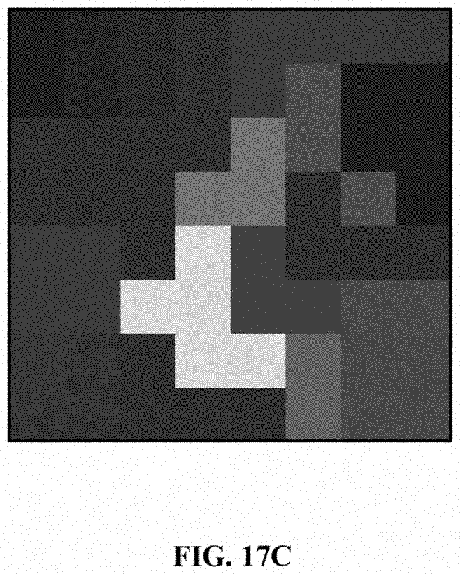

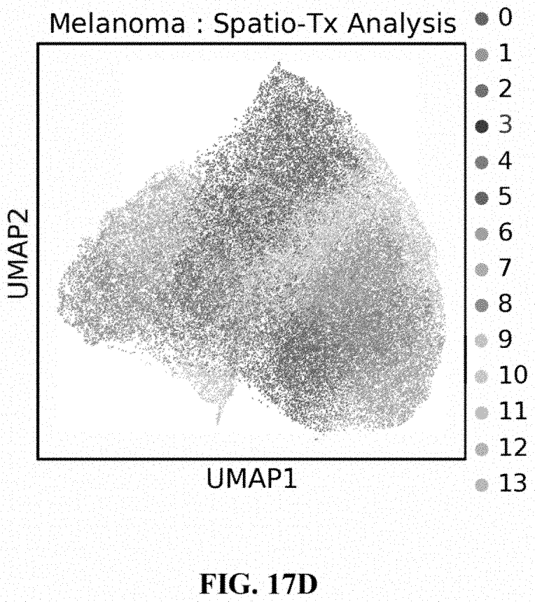

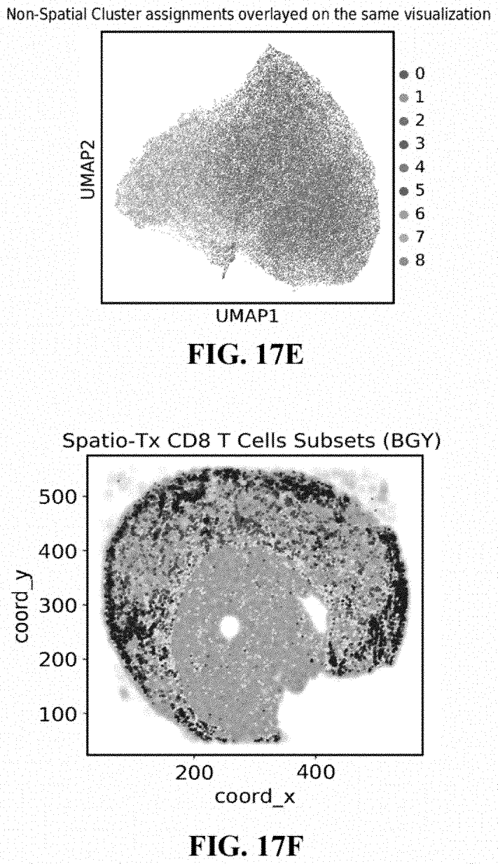

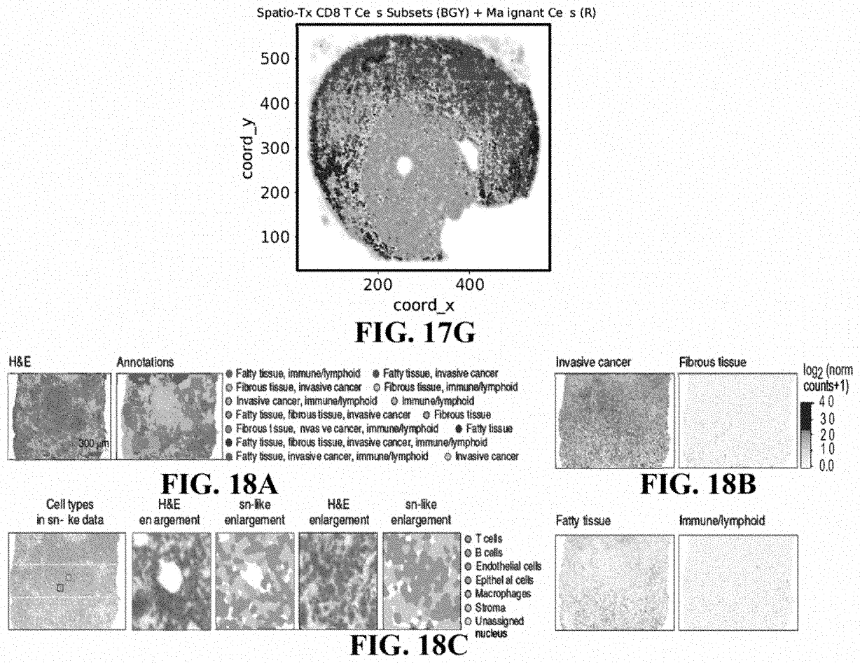

As discussed herein, a simple example from osmFISH data on pyramidal neurons using only data at the cell level and painting the resulting “group” generated a single group at layer 6. But insi2vec actually sees two clusters, which cannot be resolved from this data otherwise, but when looking at the features of these clusters, these are known subsets of neurons. FIG. 11. In the melanoma data of FIGS. 12 and 17, the insi2vec embedding was used to cluster the cells. The model allows clustering of cells only based on their intrinsic expression profiles which yields fewer clusters labeled on the same space. (FIG. 17) The CD8 T cells formed 3 clusters by insi2vec. And remarkably, these three clusters made great spatial and molecular sense, especially when shown with another cluster from the malignant cells by insi2vec. One cluster are those T cells directly proximal to MHCI+ malignant cells. A second extreme is the cluster of T cells that make their way into the cold niche, even if very sparsely (bottom panel, second from right). Critically this model generalizes across patients, like here, where trained the model on 12 patients, and then used it to group the cells in each of four other patients. Even though this is cancer and sample are not canonical, these cell features are repeatable, distinguishing cold from infiltrated cells in both CD8 T cells, malignant cells and also other immune cell content. (FIG. 13). Other applications are envisioned based on current disclosure, including the ability to evaluate differentially expressed genes that many other sc-integration methods are unable to do, see, e.g., liger.

### REFERENCES RELEVANT TO EXAMPLES 1 AND 2 ARE PROVIDED BELOW

1. E. Lein, L. E. Borm, S. Linnarsson, The promise of spatial transcriptomics for neuroscience in the era of molecular cell typing. Science. 358, 64-69 (2017).
2. E. Z. Macosko et al., Highly Parallel Genome-wide Expression Profiling of Individual Cells Using Nanoliter Droplets. Cell. 161, 1202-1214 (2015).
3. G. X. Y. Zheng et al., Massively parallel digital transcriptional profiling of single cells. Nat. Commun. 8, 14049 (2017).
4. M. Stoeckius et al., Simultaneous epitope and transcriptome measurement in single cells. Nat. Methods. 14, 865-868 (2017).
5. R. Satija, J. A. Farrell, D. Gennert, A. F. Schier, A. Regev, Spatial reconstruction of single-cell gene expression data. Nat. Biotechnol. 33, 495-502 (2015).
6. K. Achim et al., High-throughput spatial mapping of single-cell RNA-seq data to tissue of origin. Nat. Biotechnol. 33, 503-509 (2015).
7. N. Habib et al., Massively parallel single-nucleus RNA-seq with DroNc-seq. Nat. Methods. 14, 955-958 (2017).
8. S. C. van den Brink et al., Single-cell sequencing reveals dissociation-induced gene expression in tissue subpopulations. Nat. Methods. 14, 935-936 (2017).
9. E. S. Lein et al., Genome-wide atlas of gene expression in the adult mouse brain. Nature. 445, 168-176 (2007).
10. I. Tirosh et al., Dissecting the multicellular ecosystem of metastatic melanoma by single-cell RNA-seq. Science. 352, 189-196 (2016).
11. E. Lubeck, A. F. Coskun, T. Zhiyentayev, M. Ahmad, L. Cai, Single-cell in-situ RNA profiling by sequential hybridization. Nat. Methods. 11, 360-361 (2014).
12. K. H. Chen, A. N. Boettiger, J. R. Moffitt, S. Wang, X. Zhuang, RNA imaging. Spatially resolved, highly multiplexed RNA profiling in single cells. Science. 348, aaa6090 (2015).
13. J. R. Moffitt et al., High-throughput single-cell gene-expression profiling with multiplexed error-robust fluorescence in-situ hybridization. Proc. Natl. Acad. Sci. U.S.A. 113, 11046-11051 (2016).
14. F. Chen et al., Nanoscale imaging of RNA with expansion microscopy. Nat. Methods. 13, 679-684 (2016).
15. Y. Goltsev et al., Deep Profiling of Mouse Splenic Architecture with CODEX Multiplexed Imaging. Cell. 174, 968-981.e15 (2018).
16. M. Angelo et al., Multiplexed ion beam imaging of human breast tumors. Nat. Med. 20, 436-442 (2014).
17. C. Giesen et al., Highly multiplexed imaging of tumor tissues with subcellular resolution by mass cytometry. Nat. Methods. 11, 417-422 (2014).
18. X. Wang et al., Three-dimensional intact-tissue sequencing of single-cell transcriptional states. Science. 361 (2018), doi:10.1126/science.aat5691.
19. M. J. Hawrylycz et al., An anatomically comprehensive atlas of the adult human brain transcriptome. Nature. 489, 391-399 (2012).
20. S. W. Oh et al., A mesoscale connectome of the mouse brain. Nature. 508, 207-214 (2014).
21. J. Livet et al., Transgenic strategies for combinatorial expression of fluorescent proteins in the nervous system. Nature. 450, 56-62 (2007).
22. P. L. Ståhl et al., Visualization and analysis of gene expression in tissue sections by spatial transcriptomics. Science. 353, 78-82 (2016).
23. K. L. Michael, L. C. Taylor, S. L. Schultz, D. R. Walt, Randomly ordered addressable high-density optical sensor arrays. Anal. Chem. 70, 1242-1248 (1998).
24. K. L. Gunderson et al., Decoding randomly ordered DNA arrays. Genome Res. 14, 870-877 (2004).
25. B. Tepe et al., Single-Cell RNA-Seq of Mouse Olfactory Bulb Reveals Cellular Heterogeneity and Activity-Dependent Molecular Census of Adult-Born Neurons. Cell Rep. 25, 2689-2703.e3 (2018).
26. A. Zeisel et al., Molecular Architecture of the Mouse Nervous System. Cell. 174, 999-1014.e22 (2018).
27. S. Nagayama, R. Homma, F. Imamura, Neuronal organization of olfactory bulb circuits. Front. Neural Circuits. 8, 98 (2014).
28. V. Svensson, S. A. Teichmann, O. Stegle, SpatialDE: identification of spatially variable genes. Nat. Methods. 15, 343-346 (2018).
29. A. Jemt et al., An automated approach to prepare tissue-derived spatially barcoded RNA-sequencing libraries. Sci. Rep. 6, 37137 (2016).
30. J. F. Navarro, J. Sjöstrand, F. Salmén, J. Lundeberg, P. L. Ståhl, ST Pipeline: an automated pipeline for spatial mapping of unique transcripts. Bioinformatics. 33, 2591-2593 (2017).
31. P. I. Costea, J. Lundeberg, P. Akan, TagGD: fast and accurate software for DNA Tag generation and demultiplexing. PLoS One. 8, e57521 (2013).
32. K. Wong, J. F. Navarro, L. Bergenstråhle, P. L. Ståhl, J. Lundeberg, ST Spot Detector: a web-based application for automatic spot and tissue detection for spatial Transcriptomics image datasets. Bioinformatics. 34, 1966-1968 (2018).
33. J. Schindelin et al., Fiji: an open-source platform for biological-image analysis. Nat. Methods. 9, 676-682 (2012).

### Example 3—Automated HDST

ST has shown robust results on a wide variety of tissues²⁸⁻³¹ but the manual multi-step procol can be demanding. To increase the throughput, robustness, account for histopathology requirements and reduce hands on processing times to an absolute minimum, Applicants have adopted and improved the previously described ST protocol³² into an easily adjustable microfluidics processing platform. ST generates genome-wide transcriptomic data from spatially defined regions of intact tissues. A fresh frozen tissue section is placed on top of printed positional barcoded cDNA primers on a glass surface²⁰. Following tissue staining and microscopic imaging, simultaneously, the permeabilized cells release cellular RNAs and the spatially positioned primers are released directly into the tissue. The material is then converted into cDNA sequencing libraries where the RNA-seq information can be traced back to the barcode positions on the glass slide. Here, Applicants describe an improved and fully automated spatial transcriptomics platform using a commercially available^{33,34} liquid handling robotic platform. This allows for processing of 64 tissue sections and up to 96 cDNA sequencing-ready libraries in a total of ˜2 days.

### Methods

### Bravo System Requirements

Bravo Automated Liquid Handling Platform (Agilent Technologies, USA) was equipped with a 96LT pipetting head (G5498B #042, Agilent Technologies, USA) and two Peltier thermal stations (CPAC Ultraflat HT 2-TEC, #7000166A, Agilent Technologies, USA) with PCR adapter having a mounting frame at positions 4 and 6 on the Bravo Deck and connected to an Inheco MTC Controller. On position7, Applicants recommend the MAGNUM FLX™ Enhanced Universal Magnet Plate (#A000400, Alpaqua, USA) to serve for magnetic bead-based clean ups. In addition, a BenchCel NGS Workstation (Front-load rack at 660 mm height) and BenchCel Configuration Labware MiniHub (option #010, Agilent Technologies, USA) were included in the automation platform setup. In case in situ reactions were performed, the PCR adapter was removed from position 6 to be replaced with Aluminum Heat Transfer Plate (#74116-GS-4, V&P Scientific, Inc, USA).

### Sample Collection and Cryosectioning

A small piece of freshly collected tissue (˜25-50 mg, about 5×5 mm) was placed on a dry and sterile petri dish, which was placed on top of wet ice. The tissue was then very gently moved using a forceps and placed on another dry part of the petri dish to ensure little liquid was present around the tissue. The bottom of a cryomold (5×5 mm, 10×10 mm or 25×20 mm) was filled with pre-chilled (4° C.) OCT (Tissue-Tek; Sakura Finetek, USA) and the tissue transferred with a forceps into the OCT-prefilled mold. The whole volume of the tissue with pre-chilled OCT. The mold was then placed on top of dry ice and allowed the tissue to freeze for max 5 minutes until OCT has turned completely white and hard. The tissue cryomolds were stored at −80° C. until use. For cryosectioning, the ST slide and the tissue molds first reached the temperature of the cryo chamber. The OCT-embedded tissue block was attached onto a chuck with pre-chilled OCT and allow to freeze ˜5-10 min. The chuck was placed in the specimen holder and adjusted the position to enable perpendicular sectioning at 10 μm thickness. Sections were gently transferred to a ST array and then the back side of the slide was warmed ˜10-15 sec with a finger. ST slides with tissue sections on top could be stored at −80° C. for up to 6 days.

### Tissue Fixation and H&E Staining

The ST slide with the tissue section was warmed to 37° C. for 1 minute on a thermal incubator (Eppendorf Thermomixer Option C, Germany). The tissue was then covered with 4% formaldehyde (Sigma-Aldrich, USA) in 1×PBS (Thermo Fisher Scientific, USA) for 10 minutes. The whole slide was then washed in 1×PBS in a vertical orientation to be placed back on a horizontal place for drying. 500 μl isopropanol covered the tissue and ensured drying. The slide was put into a EasyDip Slide Jar Staining System (Weber Scientific) holder and the same system used for H&E staining. Five ˜80 ml containers were prepared with Dako Mayers hematoxylin (Agilent, USA), Dako Bluing buffer (Agilent, USA), 5% Eosin Y (Sigma-Aldrich, USA) in 0.45M Tris acetate (Sigma-Aldrich, USA) buffer at pH 6 and two jars with nuclease-free water (Thermo Fisher Scientific, USA). The slide rack was fully immersed in hematoxylin for 6 minutes and then washed by dipping the slide rack in a nuclease-free water jar 5 times following another destaining wash by dipping the slide rack in 800 mL nuclease-free water for 30 times. The slide rack was put into the Dako bluing buffer and incubated for 1 minute. The slide was again washed by dipping the rack 5 times in the second nuclease-free water jar. The slide rack was finally put into the eosin and incubated for 1 minute to be washed by dipping the rack 7 times in the second water jar. The slide was removed from the rack to allow it to dry.

### Automated Imaging

Images of stained tissue sections on the ST slides were taken a Metafer Vslide scanning system (MetaSystems, Germany) installed on an Axio Imager Z2 microscope (Carl Zeiss, Germany) using an LED transmitted light source and a CCD camera. All images were taken with the A-P 10×/0.25 Ph1 objective lens. A configuration program was made to enable automatic tissue detection, focusing and scanning on all ST arrays present on a glass slide. In short, tissue detection was based on contrast as compared to normalized background in RGB channels. Upon finding maximum contrast in a 12-step spiral-like search window field of view (FOV) pattern, the automated focal alignment in every second of each FOV (4000×4000 μm) was initiated. The alignment search considered the maximum contrast z-position as in-focus using 5 μm stage intervals (n=19 focal planes). The scanning of the predefined ST array areas was done in a total of 48 FOVs and ˜30 sec in 3 channels (RGB). Images were stitched using 60 μm overlap and linear blending between FOVs with the VSlide software (v1.0.0) and then extracted using jpg compression. Multiple ST slides can be processed in the same manner without any user input for a total of 6 min processing time per slide, which included image stitching ST Automation approach

The robotic protocols are divided into three main parts. They represent both an adaptation and improvement of the previously described spatial transcriptomics protocols^{20,27,32}. The first part processes all in situ reactions on a ST slides: tissue pre-permeabilization, permeabilization, reverse transcription with or without the mRNA:cDNA hybrid cleavage and tissue removal. The collected material is transferred to a standard 96-well PCR microplate (Eppendorf, Germany). All of the following reactions are run in 96-well plates. The second robotic protocol ensured the second strand synthesis reaction, cDNA bead purification, T7 in vitro transcription and a final amplified RNA (aRNA) bead purification are performed. The third and last robotic protocol includes the aRNA adapter ligation, post-ligation bead purification, second cDNA synthesis and bead purification. The material is then quantified using a standard qPCR protocol and the libraries accordingly indexed for Illumina sequencing. Reference material preparation

In order to test reproducibility of the last two parts of the automated ST protocol run in 96-well plates, Applicants prepared reference material as input. 7.5 μg of universal mouse reference RNA (#740100, Agilent Technologies, USA) was fragmented using NEBNext Magnesium RNA fragmentation module (NEB, USA) for 1 minute at 94° C. The sample was purified with a MinElute Cleanup kit (Qiagen, Germany) according to manufacturer's instructions and the RNA concentration and size assessed on a Qubit RNA HS kit (Thermo Fisher Scientific, USA) and Bioanalyzer Pico 6000 kit (Agilent Technologies, USA), respectively. About 2 μg of fragmented RNA was incubated with either 204 custom hexamer primer or poly(d)T primer in the presence of 0.5 mM dNTP (Thermo Fisher Scientific, USA) at 65° C. for 5 minutes. The hexamer primer read GACTCGTAATACGACTCACTATAGGGACACGACGCTCTTCCGATC (T7handle_IlluminaAhandle_hexamer) (SEQ ID NO: 18) and the poly(d)T primer read T7handle_IlluminaAhandle_hexamer 20TVN. First strand reverse transcription was performed with a final concentration of 1× First Strand Buffer, 5 mM DTT, 2U/μl RNaseOUT and 20U/μl of Superscript III (all from Thermo Fisher Scientific, USA). The reaction was incubated at 25° C. for 10 min (when using hexamer priming), followed by 50° C. for 1 hr and 70° C. for 15 minutes or 50° C. for 1 hr and 70° C. for 15 minutes for poly(d)T priming. The reaction was purified with AMPure XP beads (Beckman Coulter, USA) at a beads/DNA ratio of 0.8:1. The concentration of the material was measured on a Qubit RNA HS kit (Thermo Fisher Scientific, USA) and diluted in elution buffer (Qiagen, Germany) to 0.25 ng/ul. A release mixture of 0.75 ng first strand cDNA, 1× Second strand buffer (Thermo Fisher Scientific, USA), 0.2 μg/ul BSA and 0.5 mM dNTP (Thermo Fisher Scientific, USA) was prepared.

### In Situ Robotic Protocol

Input to this part of the protocol are tissue-stained ST slides. The ST slide is attached ProPlate Multi-Array slide system (GraceBioLabs, USA). Up to four ST slides are fitted into one ProPlate Multi-Array slide system (GraceBioLabs, USA). The ProPlate Multi-Array system is then fixed in position by Aluminum Heat Transfer Plate (VP 741I6-GS-4, V&P Scientific, Inc, USA) on the Agilent Bravo deck. The protocol starts with tissue pre-permeabilization (20 min for human colon and 30 min for mouse brain) with addition of 120 μl reagent per well of either 2.5 U/μl liberase (human colon; Sigma-Aldrich, USA) in 1× Hank's Buffered Salt Solution (Thermo Fisher Scientific, USA) with 0.2 μg/ul BSA or exonuclease I buffer (mouse brain; NEB, USA) can be used. For complete removal of the reagents and wash solutions from the subarrays all of the robotic dispensing and aspiration steps takes place in all four corners of the square wells. Pre-permeabilization reagent removal is followed by a 100 μl wash in 0.1× Saline Sodium Citrate (SSC, Sigma-Aldrich, USA). Next, tissue permeabilization takes place with 75 μl 0.1% pepsin (pH 1, Sigma-Aldrich, USA) for 10 min. After a 100 μl, 0.1×SSC wash, in situ cDNA synthesis reaction is performed by the addition of 75 μl RT reagents: 50 ng/μl actinomycin D (Sigma-Aldrich, USA), 0.5 mM dNTPs (Thermo Fisher Scientific, USA), 0.19 μg/μl BSA (NEB, USA), 1× First strand buffer, 5 mM DTT, 2U/μl RNaseOUT, 20U/μl Superscript III (all from Thermo Fisher Scientific, USA). The reactions are sealed with 70 μl of white mineral oil Drakerol #7 (Penreco, USA). Incubation at 42° C. is performed for a minimum of 6h, then the reaction mix is removed followed by an 0.1×SSC wash of the slide surface. In case of the highly efficient ST protocol, the in situ cDNA synthesis mix was supplemented with the following: 1 U/μl USER enzyme (NEB, USA), 6% v/v lymphoprep (STEMCELL Technologies, Canada) and 1M betaine (#B0300-1VL, Sigma-Aldrich, USA). In case a Cy3 fluorescent cDNA activity print is needed for tissue optimization, the 75 μl in situ cDNA reaction mix was as follows: 50 ng/μl actinomycin D (Sigma-Aldrich, USA), 0.19 μg/μl BSA (NEB, USA), 1× M-MuLV buffer, 5 mM DTT, 2U/μl RNaseOUT, 20U/μl M-MuLV (all from Thermo Fisher Scientific, USA), 2.4 μl dNTP mix (dATP; dGTP and dTTP at 10 mM and dCTP at 2.5 mM) and 1.2 μl Cy3-dCTPs (0.2 mM, Perkin Elmer, USA).

The next part of the protocol encompasses tissue removal and takes place in two separate steps with RLT buffer with β-mercaptoethanol and Proteinase K. Depending on the tissue type a selection of a one-step or two-step protocol can be chosen. The β-mercaptoethanol mixture with RLT buffer is prepared in the reagent plate with 50 ul of mineral oil on top to avoid leakage of β-mercaptoethanol smell. 200 μl of the mixture is added to the wells and incubated at 56° C. for 1 h. Following removal of reaction mix and wash with 0.1×SSC solution, 200 μl of second tissue removal mixture; 2.5 μg/μl Proteinase K in PDK buffer (Qiagen, Germany); was added and the reaction was performed at 56° C. for 1 h. The complete reaction mix is again removed and efficient leftover white oil removal is accomplished with one 10 minute wash of the wells with 2×SSC/0.1% SDS (Sigma-Aldrich, USA) followed by 1 minute wash with 0.2×SSC and finally 0.1×SSC. In case of comparison to standard ST protocol, cleavage of probes from the surface was performed in the next steps and not during in situ cDNA synthesis. The reaction mix consists of: 1.1× Second strand buffer (Thermo Fisher Scientific, USA), 0.088 mM dNTPs and 1 U/μl USER enzyme (NEB, USA). 75 μl of the mix is added and the reactions sealed with 70 μl of the white mineral oil. The incubation is done for 3h at 37° C. The released material is then transferred to a new 96-well PCR plate (Eppendorf, Germany) by aspirating 70 μl of the released material underneath the oil with a multichannel pipette to avoid any sample loss during transfer. Library preparation (1)

Upon initiating the Agilent Bravo form the user is prompted to select either: 1, 2, 3, 4, 6 or 12 columns of the 96-well plate to run. Two positions on the Bravo deck should have Peltier thermal stations (4-95° C.) in the standard 96-well format. A reagent plate is prepared for the robotic aspiration, transfer and dispensing of reagents as outlined in figures, showing the layout for a 12 columns (96 samples) run. The dead volume of the reagents are ˜6-8 ul per well for a 12 column plate of samples and should be accounted for when preparing the reaction plate. First, single-stranded cDNA is made to double-stranded material using 5 ul the reaction mix (2.7 μl First strand buffer, 3.7 U/μl DNA polymerase I and 0.43 U/μl Ribonuclease H (all from Thermo Fisher Scientific, USA) for 2h at 16° C. Thereafter, the material is blunted by the addition of 5 μl of 3U/μl T4 DNA polymerase (NEB, USA) for 20 minutes at 16° C. The reaction is stopped by addition of Invitrogen UltraPure 0.5M EDTA (pH 8.0, Thermo Fisher Scientific, USA) to a final concentration of 16 μM. The material was purified using Ampure XP (Beckman Coulter, USA) purified at a bead to cDNA ratio of 1:1. Next, 27.8 μl of the T7 reaction mix (46.2 mM rNTPs, 1.5× T7 reaction buffer, 1.54 U/μl SUPERaseIN inhibitor and 2.3 U/μl T7 enzyme; all from Thermo Fisher Scientific, USA) is added and sealed with 40 μl of Vapor-Lock oil (Qiagen, Germany) for an overnight 14h incubation at 37° C. After incubation, 2.1 μl of nuclease-free water (Thermo Fisher Scientific) is added and the Vapor-Lock is removed. A bead cleanup with RNAclean Ampure XP beads (Beckman Coulter, USA) at a ratio of 1.8:1 of beads:aRNA. The material can be assessed with an Bioanalyzer RNA 6000 Pico kit (Agilent Technologies, USA). 8 μl of the eluted 141 aRNA is transferred into a new 96-well PCR plate (Eppendorf, Germany).

Library Preparation (2)

2.5 μl 3 μM aRNA adapters [rApp]AGATCGGAAGAGCACACGTCTGAACTCCAGTCAC[ddC] (SEQ ID NO: 19) are added to 8 μl of aRNAs. The reaction is then incubated at 70° C. in a PCR machine for 2 min and immediately chilled on wet ice. The user now again selects the number of columns they wish to run. 4.5 μl T4 RNA ligation mix (1× T4 RNA ligase buffer, 300U truncated T4 ligase 2 and 60U murine RNAse inhibitor (all from NEB, USA) is added to the aRNA/adapter solution. The ligation reaction takes place at 25° C. for 1 h. In case of the high-efficiency protocol, the ligation reaction is performed for 3h with in the presence of 5× aRNA adapters. The ligation is followed by a Ampure XP (Beckam Coulter, USA) bead purification at a ratio of 1.8:1 bead:cDNA and eluted in 12 μl. First, 20 of a primer and dNTP mix (1:1 v/v of 20 mM GTGACTGGAGTTCAGACGTGTGCTCTTCCGA (SEQ ID NO: 20) (20 uM) and 10 mM dNTPs) is added to the ligated samples. In case of the highly-efficient ST protocol, 5× primer amount is added using the same volumes. Then, the samples are sealed with 40 μl Vapor-Lock (Qiagen, Germany) and heated to 65° C. for 5 min. The Vapor-Lock is removed and 80 of reverse transcription mix added (0.9× First strand buffer, 4.5 mM DTT, 1.8 U/μl RNaseOUT and 9 U/μl Superscript III; all from Thermo Fisher Scientific, USA), with the addition of 40 μl Vapor-Lock to reseal the reaction. The samples are incubated at 50° C. for 1 h. 100 of nuclease-free water is added followed by a final Ampure XP bead purification at 1.7:1 bead:cDNA ratio with elution is 150 nuclease-free water.

### Quantification, Indexing and Sequencing

qPCR library quantification and indexing are performed as described in Salmén et al³². The indexed libraries are diluted with 40 μl of nuclease-free water to allow for a final library bead clean up with 0.8:1 ratio Ampure XP beads to PCR products as according to manufacturer's protocol. Final elution is done in 160 elution buffer (Qiagen, Germany). The individual libraries are evaluated on a Bioanalyzer HS or DNA 1000 (Agilent Technologies, USA), DNA1000 Tapestation (Agilent Technologies, USA) and DNA HS Qubit assays (Thermo Fisher Scientific, USA), respectively. Dilute the samples to the desired concentration for sequencing (˜1.08 pM final for NextSeq sequencing with 10% PhiX). The samples were sequenced 30nt in the forward read and 55nt in the reverse read.

### Raw Reads Processing and Mapping

Fastq reads were generated with bcl2fastq2. ST Pipeline v.1.3.1 was used to demultiplex the spatial barcodes and collapse duplicate UMI sequences. In short, 5nt trimmed R2 was used for mapping to the mouse genome (mm10) using STAR′. After that, mapped reads were annotated using HTseq-count⁴¹. To collapse UMIs, the annotated reads needed to first be connected to a spatial barcode using a TagGD⁴² demultiplexer (k-mer 6, mismatches 2). Then, UMIs mapping to the same transcript and spatial barcode were collapsed using naive clustering with one mismatch allowed in the mapping process. The output file is a genes-by-barcode matrix that was used in all further processing steps.

### Automated Image Processing for Spatial Transcriptomics

For efficient processing, HE images were scaled to approximately 500×500 pixels using the imagemagick mogrify command as follows: mogrify-define jpeg:size=500×500-resize 8%-quality 100%% RE image.jpg. In order to reconstruct the positions of all ST spots, visible (i.e., not covered by the tissue section) barcode (x,y) spots were registered through “blob detection” and then refined by keeping only those “blobs” (potential grid points) that were likely to be part of a regular grid. A regular grid was then fitted to the remaining potential grid points, starting an iterative process in which the 0.1% potential grid points that least fit the grid were removed in each iteration and a new grid was fitted until the target number of grid points per row (here 35) and column (here 33) were reached. Finally, those grid points that overlapped the tissue sections were identified by building a mask that represented the tissue area and registering all grid points that were present in this mask. In order to accommodate atypical tissue coloring, bubbles and smears present as imaging artifacts, Applicants introduced a parameter that toggles the color channels used to detect the tissue section. Finally, an intermediate report notifies the user of irregularities in the automatic alignment process and allows for visual inspection. The output .tsv file contains barcode spots (x,y) as centroid pixel coordinates of the detected grid, as well as a TRUE/FALSE value if the barcode spot is detected as under the tissue section area (i.e. TRUE).

SpoTter Integration with ST Pipeline and Quality Control (QC) Reporting

The following steps integrate the output from the automated image alignment steps with the output gene-by-barcode expression file as produced by the ST Pipeline v.1.3.1. The barcode (x,y) spots approximated as under the tissue section are used for subsetting the ST Pipeline gene-by-barcode file. Then, the original HE images are downscaled and cropped using the following imagemagick commands: convert HE_image.jpg-crop width“x”height+xa+ya; where width and height represent the euclidean lengths between (x,y) grid detected barcode spots c(33,35), c(1,35) and c(1,35), respectively. xa and ya are described as the centroid pixel coordinates of the grid point c(33,35). The cropped HE image is then rotated as follows: mogrify-flop-flip HE_image.jpg and this image is used as input to the QC reporting system and for the GUI annotation tool. A final quality control (QC) report is created when running SpoTteR. The report contains the following information:
date and time metadata for QC report creation
ST pipeline version
Raw input reads
Trimming loss
Unique mapping
Annotated reads
UMIs
Genes
Library saturation
HE Tissue Image
Heatmap of log 2(raw expression) associated with all 1007 ST barcodes
Heatmap of log 2(raw expression) associated with ST barcodes under tissue
Violin plots of UMIs outside or within tissue-detected boundaries
Mean number of transcripts per feature under tissue
Mean number of genes per feature under tissue
Number of barcode spots covered
Heatmap of log 2(raw expression) associated with top 5 genes in your ST experiment
Heatmap of log 2(raw expression) associated with 5 interesting genes in your ST experiment
   Comparison of SpoTter Vs. ST Spot Detector Vs Manual Alignment

As to compare the automated image processing developed here, Applicants needed to acquire an additional image of the ST array area after the experiment was performed and the tissue had been removed from the array surface. Briefly, complementary and Cy3 labeled oligonucleotides (IDT) were diluted in 2×SSC with 0.05% SDS to a final concentration of 1 μM. 50 μl of the diluted solution was added to the array surface and incubated with shaking (50 rpm) for 10 min at RT. This was followed by washing the slide in 4×SCC with 0.1% SDS and 0.2×SSC. The array frame and all ST barcode positions have now efficiently been labeled and could be acquired on the same imaging system as described before but now using a fluorescent light source and a FITC filter.

All input images in the following comparisons were the same approximate input sizes and resolution. Further, all plotting functions during processing have been disabled and only time needed to process the final output file with ST barcode spot under tissue was considered in the comparisons. The ST spot detector tool previously developed³⁶ uses the H&E and Cy3 images as input. Due to its intrinsic scaling factor and input image size requirements, initial pre-processing of both images was needed i.e., images needed to be linearly downscaled to 30% of their original size and both images needed to be individually cropped as to represent the same FOVs as collected during the imaging step. Applicants do note that the cropping is needed only if the user did not have the possibility to automatically acquire the same FOVs using the same staring (x,y) positions. For manual alignment, Applicants used Adobe Photoshop for initial pre-processing, same is in the previous step. Both H&E and Cy3 acquired images were downscaled to 30% its original sizes, rotated 180 degrees and aligned to the same starting (x,y) pixel coordinates. This was followed by cropping both images along the middle of the first and last row and column. The tissue boundaries were detected using the magic wand function (32px) and the selected subtracted in the Cy3 image. Spots boundaries were again detected using the same magic wand function and the background noise cleaned up using the bucket fill function (250px) in a grayscale image. This grayscale image was further used in Fiji⁴³ to detect the centroid coordinates of each ST barcode spot. For Fiji, Applicants made a macro plugin bellow:

```
# read in input and output directories through gui
input = getDirectory(“Input directory”);
output = getDirectory(“Output directory”);
suffix = “spotsjpg”; //you only want to apply to spotsjpg images that are grayscale
processFolder(input);
function processFolder(input) {
  list = getFileList(input);
  for (i = 0; i < list.length; i++) {
    if(File.isDirectory(input + list [i])) //if it's a directory, go to subfolder
    processFolder(“ + input + list[i]);
  if(endsWith(list[i], suffix)) //if it's a jpg image, process it
        processFile(input, output, list[i]);
          close(); //close image
      //if it's neither a tiff nor a directory, do nothing
  }
}
function processFile(input, output, file) {
  print(“Processing: ” + input + file);
  open(input + file); //open image
    setAutoThreshold(“Default”);
    //run(“Threshold. . .”);
    setAutoThreshold(“Default”);
    //setThreshold(0, 143);
    setOption(“BlackBackground”, false);
    run(“Convert to Mask”);
    setThreshold(255, 255);
    run(“Set Measurements. . .”, “centroid redirect=None decimal=3”);
    run(“Analyze Particles. . .”, “size=3000-Infinity show=Overlay display”);
    saveAs(“results”, output + file + “spots.tsv”); #output tsv file with ST spot centroids
    run(“Clear Results”);}
```

Following Fiji processing, (x,y) pixel centroid coordinates were translated to ST barcode spot coordinates (as given during the demultiplexing step in the ST pipeline). Image attributes i.e., image width and height were divided by 32 and 34 respectively making a scaling factor. Then, each centroid pixel coordinate from Fiji processing counting be divided by the scaling factor and rounded to the nearest digit. This (x,y) now was using the same coordinate system and scaling as the ST (x,y) pipeline files. For input to SpoTeR, Applicants only needed the original H&E imaged as acquired by the imaging system and no sGUI-based preprocessing was needed. For speed comparisons, total time need for preprocessing steps was measured first. Pre-processing steps in case of “manual” processing included alignment of the H&E and Cy3 images with Adobe Photoshop 2019 and creation of a ST array spots files. In case of ST Detector pre-processing time, Applicants could only time needed to open the same images in Adobe Photoshop, downscale them to 30% size and crop them the same size without any other image handling processes performed. For SpoTteR, preprocessing included the downscaling step performed with imagemagick. Processing steps were then performed, and time measure as described before. Total speed was considered as 1/t [s-¹] where t represents the sum of time needed for both the pre-processing and processing steps. False positive and negative rates were calculated as percentage of spots present or absent in SpoTteR or ST Detector but not it manually processed ST barcode spot coordinates as compared to all positions detected in either of the datasets.

### Estimating Lateral Diffusion

Two consecutive mouse cortex fresh frozen sections were processed. One was processed manually as described earlier³² while the other was processed using our devised robotic set up. Both the H&E and gene activity Cy3 images were processed in Fiji⁴³. Cell boundaries were detected and 10% signal intensity and these were used as breakpoints to estimate Cy3 signal diffusions i.e. lateral diffusion. Left and right cell boundaries representing opposite sides of each cells were used in the estimate and a total of 9 cells used each condition, although more cells can be utilized. A pixel to distance conversion ratio was used. If a diffusion distance measure was scored as negative it implied that the Cy3 signal was contained within the detected cell boundaries, and positive if outside those same boundaries. For comparing results between the condition, Applicants used only those values scored positively and significance comparison was performed using a t-test.

### Image Annotation

To manually annotate tissue images based on their H&E features, Applicants used a previously adapted graphical and cloud-based user interface²². Applicants assigned each ST (x,y) coordinate with one or more regional tags. The region names used were: Olfactory Nerve Layer (ONL), Granular Cell Layer (GCL-E), Granular Cell Layer Internal (GCL-I), Deep Granular Zone (GCL-D), External Plexiform Layer (EPL), Mitral Layer (M/T), Internal Plexiform Layer (IPL), Subependymal Zone (SEZ), Granular Cell Layer (GL), Cortex (CTX) and Auxiliary Olfactory Bulb (AOB). For comparisons between ST2.5 and manually prepared libraries, as well as Splotch, regions were merged as following:

| | |
|---|---|
| Granula Cell Layer Deep (GCL-D) | GR |
| Glomerular Layer (GL) | GL |
| Granule Cell Layer External (GCL-E) | GR |
| Granule Cell Layer Internal (GCL-I) | GR |
| Subependymal Zone (SEZ) | GR |
| Internal Plexiform Layer (IPL) | IPL |
| External Plexiform Layer (EPL) | OPL |
| Mitral Layer (M/T) | MI |
| Olfactory Nerve Layer (ONL) | ONL |

### Comparisons Between Gene Expression Profiles

For comparisons between the ST2.5 and manual datasets, all data were first downsampled to the same saturation level (64%) before invoking a ST pipeline mapper, annotator and counter run to receive UMIs per spatial (x,y) barcode as described previously. Depending on the sequencing depth, a gene was counted as expressed if the corresponding transcript was present in >1, >3 and >40 copies (when analyzing samples at raw sequencing depths of 10,000,000; 30,000,000 and 400,000,000 reads, respectively). The total count over all spots per gene and sample were normalized using a naive transformation⁴⁴. Pearsons's correlation coefficient between the average and normalized samples was calculated using Scipy v1.2.0⁴⁵.

### Saturation Curve Generation

Number of unique molecules were calculated by first sub sampling the same proportion of annotated reads from each sample and then run the samples through ST Pipeline v.1.3.1, where unique molecules were calculated as previously described.

Spatial Gene Expression Analysis—Splotch

Statistical analysis of the spatial gene expression data was performed using Splotch two-level hierarchical model (https://github.com/tare/Splotch) as previously described³¹. In short, the model captures gene expression in anatomical regions by taking into account experimental parameters such as, in our case, different enzymatical conditions and concentrations and calculates gene expression for single genes per annotated spot, as well as differential expressed genes per region captured in Bayesian factors (BF) using Bayesian interference with Hamiltonian Monte Carlo. To find genes which were differential expressed in, as an example, the annotated region ONL compared to the other regions, Applicants used Splotch to compute the BF{ONL vs. Other regions}.

### Comparison to Allen Brain Atlas Data

To validate the findings, Applicants downloaded ISH gene expression data from five regions: GL (Glomerular Layer), GR (Granule Cell Layer), IPL (Internal Plexiform Layer), MI (Mitral Layer) and OPL (External Plexiform Layer), from the Allen Brain Atlas (ABA). To be able to compare our samples with the ABA reference and since Applicants had annotated our samples in more detail, Applicants merged our regions before Splotch as previously described. Auxiliary Olfactory Bulb (AOB) and Cortex (CTX) were excluded from the Splotch analysis. Applicants filtered for genes with fold change more than a particular cutoff in ABA, compared to genes with positive fold change and log 10(BF) more than an identified parameter in our Splotch data and computed a one-sided Fisher's exact test using Scipy v1.2.0⁴⁵. Resulting p values were corrected for multiple testing using Benjamini/Hochberg. One of the top most differential expressed genes in both ST2.5 and ABA were chosen from each region and its gene expression in all samples were visualized. The visualizations were compared to the corresponding in situ hybridization (ISH) and fluorescent images, downloaded from ABA webpage (https://mouse.brain-map.org/). In addition, ST2.5 was compared to ST samples (Ståhl et al. 2016). This ST dataset was also analyzed using Splotch with the same settings as used for ST2.5, before visualized and compared to ST2.5. Genes which were not found in ST samples, but found in ABA, were finally visualized.

### Code Availability

All code has been deposited on GitHub at klarman-cell observatory/staut (https://github.com).

### Data and Materials Availability

The data have been deposited to NCBI's GEO archive GSE. All processed data is available at the Single Cell Portal (https://portals.broadinstitute.org).

### Results

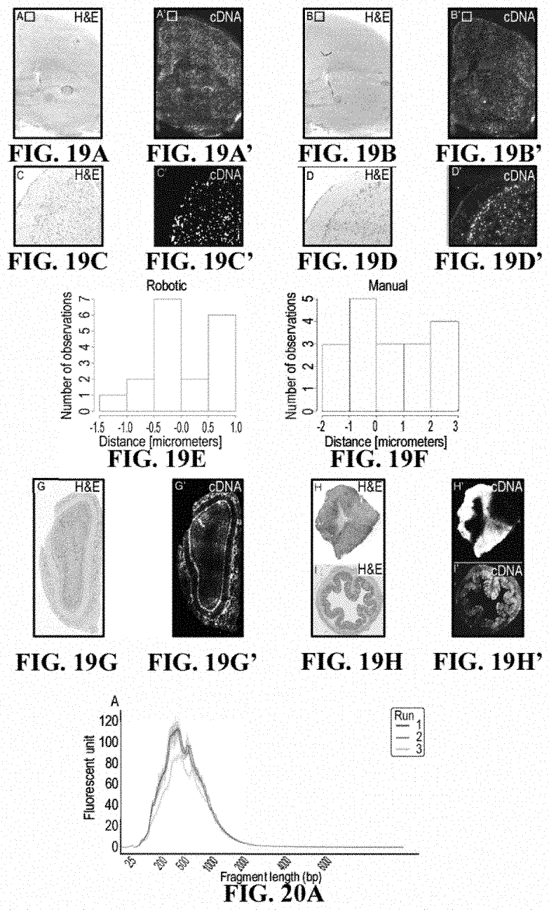

Applicants tested the automated platform in two separate occasions: (1) in situ and (2) library preparation reactions. The in situ tissue processing was done using a ProPlate Multi-Array slide system (GraceBioLabs) and a low-cost adapter (Methods). In addition, the in situ tissue processing can be run in “optimization mode” or “library preparation mode”. Optimization mode gives the user information on tissue permeabilization effects where a Cy3 fluorescent print of spatial cDNA activity is created and measured³². The localized cDNA footprint is compared to the histological H&E pattern and the extent of molecular lateral leakage outside the tissue boundaries measured. Applicants confirmed that using the automated platform allowed for recreation of the spatial fluorescent patterns in four tested tissues: cortex and main olfactory bulb of mouse brain, distal mouse colon and a preclinical model of colorectal cancer (FIG. 19A-19H′). With these results, estimated lateral diffusion was 0.5 um, which confirms weakened lateral diffusion (p<0.01, Mann-Whitney) 3× lower compared to previous experiments^{20,32,35}.

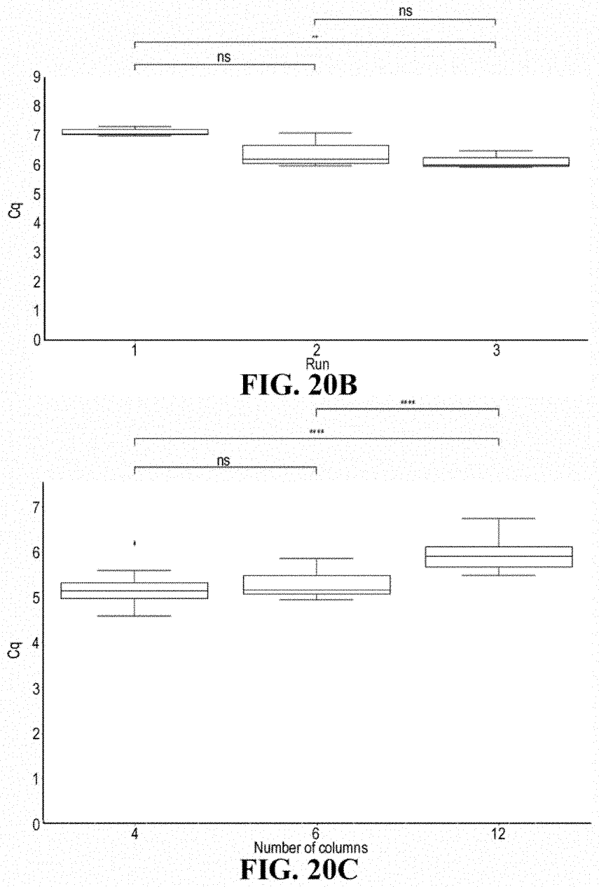

Library preparation mode gives the user 3′ spatial RNAseq information. When running the library preparation mode, three main steps are performed: (1) in situ reactions as according to optimized tissue conditions; (2) second strand synthesis and in vitro transcription and (3) adapter ligation with cDNA synthesis. Given positive results in optimization mode, Applicants first sought to evaluate the performance of (2) and (3). These reactions are also scalable by user-input i.e. the user can choose to run anywhere between 1 and 96 samples in parallel in 8-step increments with adjusted consumable usage to alleviate costs. Using fragmented reference cDNA material as input (Methods), no significant variation (p value>0.05) was shown between 3 separate library preparation runs (FIGS. 20A-20B). Additionally, no significant variability was shown within each run or user-defined throughput set up (FIG. 20C).

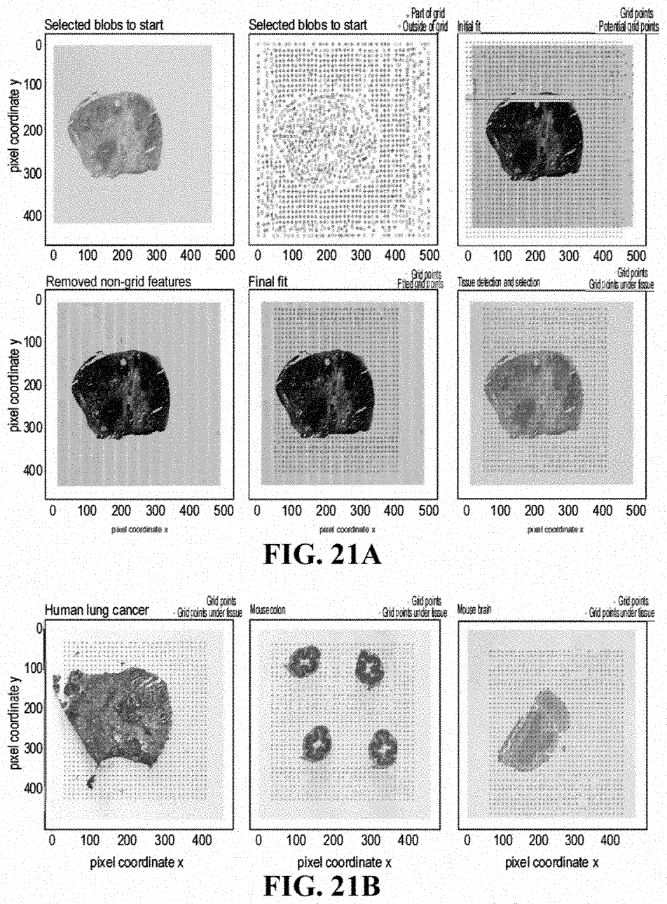

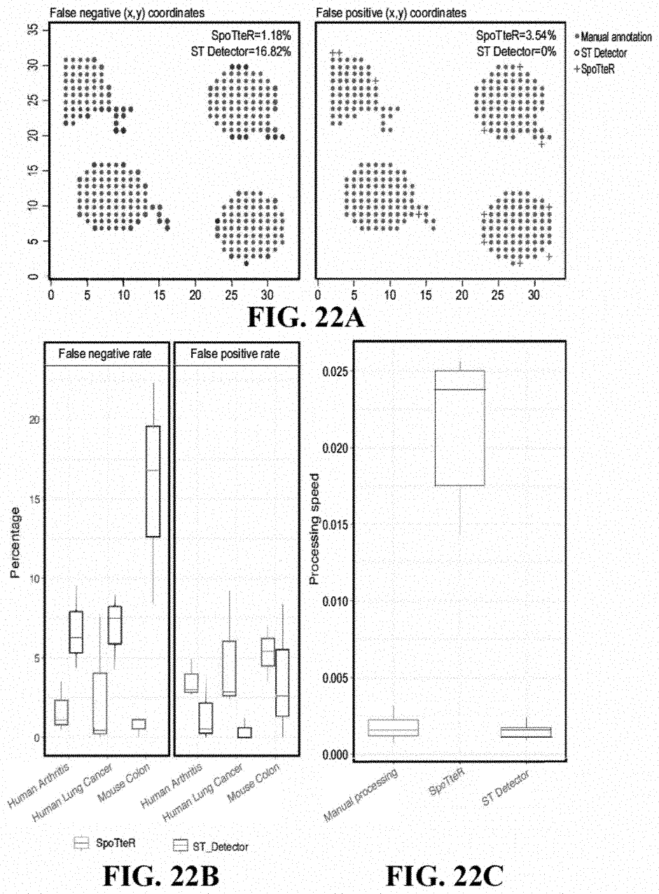

Finally, Applicants tested the performance of the fully automated set as compared to that prepared manually. To this end, Applicants also developed a fast and fully automated end-to-end ST image integration method termed SpoTteR. With SpoTteR, images are automatically downscaled to ensure fast processing and barcode spots positions reconstructed using iterative blob detection and grid fitting (Methods, FIG. 21A). The approach accounts for various imaging artifacts present, such as uneven tissue coloration, background slide smear effects and pipetting bubbles. Finally, tissue's coordinates are also registered through a masking process and this automatic alignment approach combined with the sequencing data to make a gene-by-barcode matrix. Further, SpoTteR creates a first quality report system for spatially resolved data.

To test SpoTteR's performance, Applicants compared its detection rates and processing speed to manual and semi-automated approaches 36 previously described. The results show that SpoTter is agnostic tissue type and size when detecting and assigning barcodes spots to a predefined grid (FIG. 21B). Compared to the semi-automated approach, no user interaction is needed either during the image pre-processing or ST barcode detection steps making our fully automated approach up to 14× faster while keeping 96.46% false positive and 98.82% false negative accuracy, while the semi-automated approach results in high false negative errors (FIG. 22A-22C). Now, Applicants could easily annotate the H&E images using a GUI so that each ST (x,y) expression spot is assigned with one or more of 11 different morpho-regional tags (Methods).

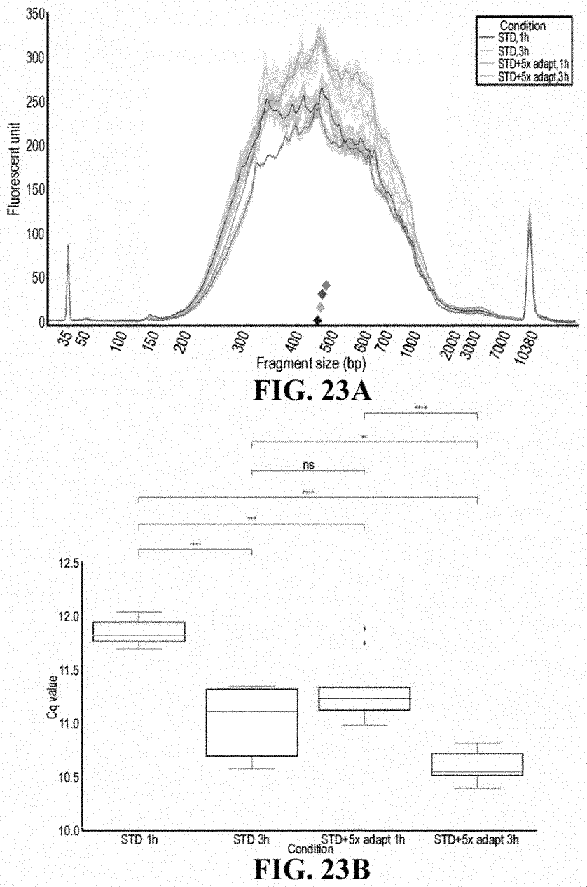

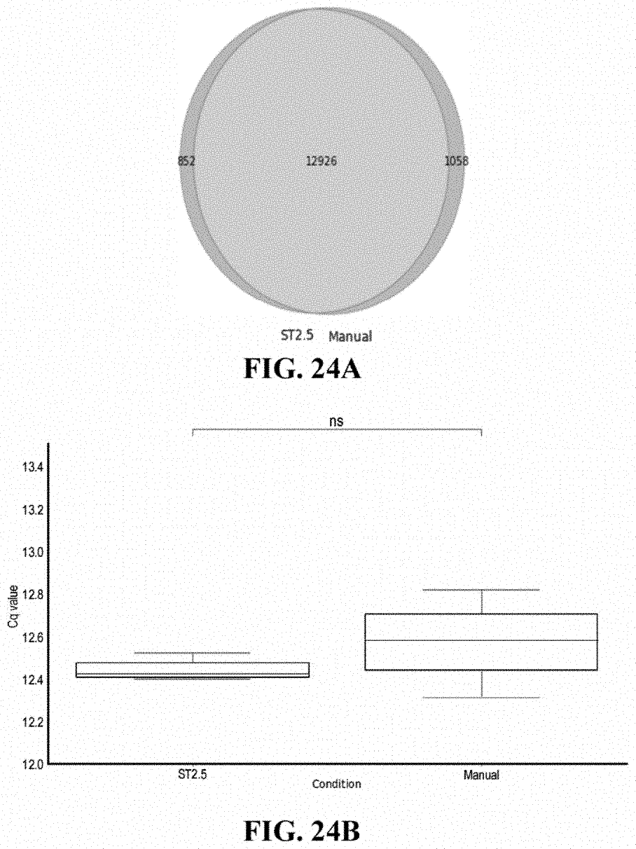

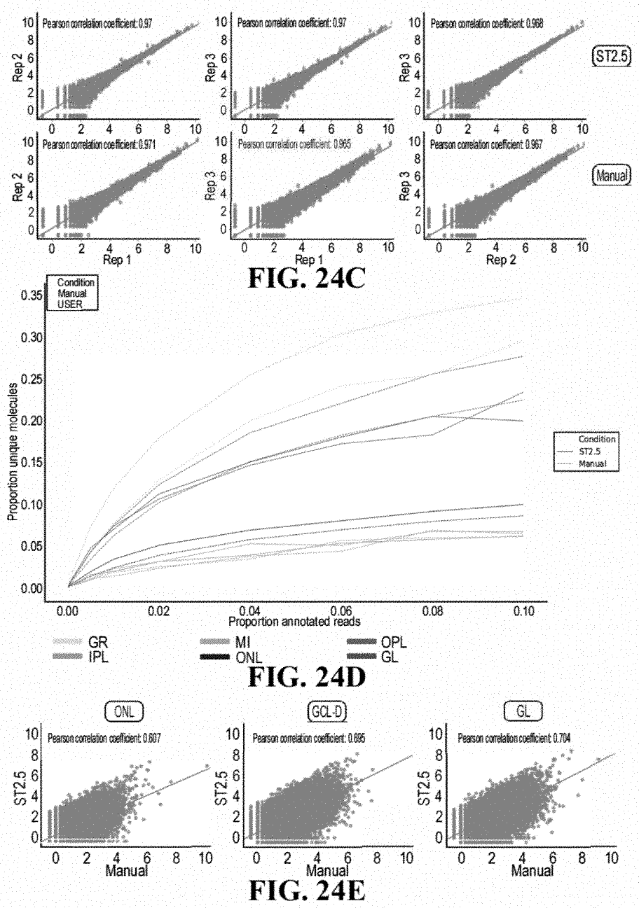

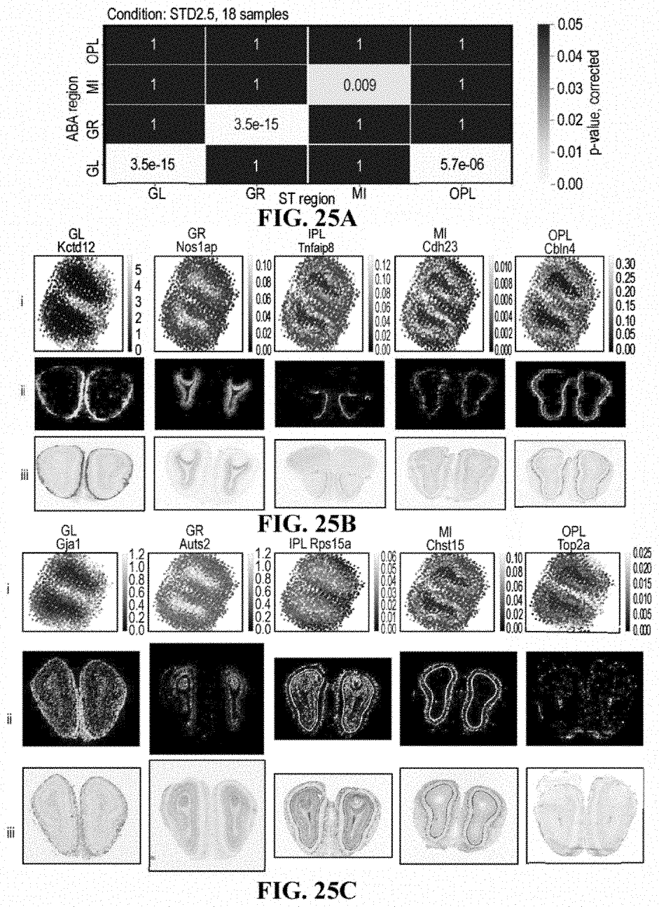

When comparing ST2.5 vs. manual protocol performances, the majority of the genes agreed between the two preparations (FIG. 23A). Further, the two setups have on average similar expression profiles at the same sequencing depth (FIG. 23B) and also gave similar average sensitivity (defined as the total number of unique molecular identifiers; UMIs) for each morphological region (FIG. 23C). These results confirmed excellent reproducibility within and between automated runs while keeping the spatial specificity and sensitivity as compared to the standard manually prepared ST protocol. Next Applicants explored whether sensitivity could be increased i.e. number of genes and UMIs detected per ST (x,y) coordinate. Previous reports noted ST sensitivity at 6.9±1.5% to that of single-molecule fluorescent in situ hybridization20. Here, Applicants report sensitivity optimizations in three steps. The first major change includes a parallel capture of mRNA molecules onto the releases barcode cDNA primers otherwise present on the ST array surface (Methods). In short, Applicants reasoned that upon mRNA hybridizing to the poly(d)T capture probes on the ST slide surface, that hybrid is stable and can be used as a template for a reverse transcription reaction in solution. To ensure that, the hybrid also needed to be released from the slide surface using a restriction site close to the 5′ end of the surface capture probes. Now, a parallel and supplemented cDNA synthesis reaction (Methods) could be performed on the slide's surface and the total processing time was decreased from ˜1.5 days to ˜6h. To further increase efficiency, Applicants adjusted the amount of adaptors and reaction time in the subsequent ligation steps during library preparation. Applicants report no difference in library length but a significant increase in library outputs present after either of these two optimizations performed (FIG. 24A-24B). After sequencing, the total number of protein-coding genes increased (FIG. 24C) as compared to the standard protocol. UMI-based sensitivity showed a linear increase in correlation to sequencing depth and protocol (FIG. 24D) marking a significant increase in sensitivity (p-val). Average expression profiles between three profiled sections agreed significantly (FIG. 24E). Compared to previous results, efficiency can be estimated to that of smFISH. Next Applicants asked the question whether one can detect correct spatial gene expression using ST2.5. Splotch31,37 was used to align our replicate tissue sections per condition and generate posterior estimates of spatial gene expression and evaluate spatial autocorrelation. After running Splotch, Applicants confirmed that region-enriched and upregulated genes (beta>2) were present in the correct spatial regions as compared to expression estimated provided in the Allen Brain Atlas38 (Methods, FIG. 25A-25B). When comparing spatially variable genes in the ST and ST2.5 approaches, Applicants capture the spatial variation as expected with ST (FIG. 25C) but add new spatial targets using ST2.5.

Throughput and robustness are needed to transition away from current limitations of low replication spatial genomics profiling. Namely, volumetric sampling requires vast number of tissue sections to be processed to make biological discoveries31,39. Robotization on widely used platform enables use of appropriate study design and replication while minimizing technical variation. In addition, it enables laboratories with very limited training to adapt new technologies into their sample processing pipelines. ST2.5 is a highly efficient and automated workflow for spatially resolved transcriptomics, easily adaptable to new ST array versions and designs. ST2.5 does not rely on any customized microfabrication, uses commercially widely-available liquid handlers with minimum preparation time per run (˜30 min), has an end-to-end image-integrated data analysis pipeline and is readily deployable to the wide scientific community.

### REFERENCES UTILIZED IN EXAMPLE 3

1. Hawrylycz, M. J. et al. An anatomically comprehensive atlas of the adult human brain transcriptome. Nature 489, 391-399 (2012).
2. Oh, S. W. et al. A mesoscale connectome of the mouse brain. Nature 508, 207-214 (2014).
3. Livet, J. et al. Transgenic strategies for combinatorial expression of fluorescent proteins in the nervous system. Nature 450, 56-62 (2007).
4. Lein, E., Borm, L. E. & Linnarsson, S. The promise of spatial transcriptomics for neuroscience in the era of molecular cell typing. Science 358, 64-69 (2017).
5. Zheng, G. X. Y. et al. Massively parallel digital transcriptional profiling of single cells. Nat. Commun. 8, 14049 (2017).
6. Macosko, E. Z. et al. Highly Parallel Genome-wide Expression Profiling of Individual Cells Using Nanoliter Droplets. Cell 161, 1202-1214 (2015).
7. Klein, A. M. et al. Droplet barcoding for single-cell transcriptomics applied to embryonic stem cells. Cell 161, 1187-1201 (2015).
8. Zeisel, A. et al. Molecular Architecture of the Mouse Nervous System. Cell 174, 999-1014.e22 (2018).
9. Lake, B. B. et al. Neuronal subtypes and diversity revealed by single-nucleus RNA sequencing of the human brain. Science 352, 1586-1590 (2016).
10. van den Brink, S. C. et al. Single-cell sequencing reveals dissociation-induced gene expression in tissue subpopulations. Nat. Methods 14, 935-936 (2017).
11. Chen, K. H., Boettiger, A. N., Moffitt, J. R., Wang, S. & Zhuang, X. RNA imaging. Spatially resolved, highly multiplexed RNA profiling in single cells. Science 348, aaa6090 (2015).
12. Lubeck, E., Coskun, A. F., Zhiyentayev, T., Ahmad, M. & Cai, L. Single-cell in situ RNA profiling by sequential hybridization. Nature methods vol. 11 360-361 (2014).
13. Eng, C.-H. L. et al. Transcriptome-scale super-resolved imaging in tissues by RNA seqFISH. Nature 568, 235-239 (2019).
14. Lee, J. H. et al. Highly multiplexed subcellular RNA sequencing in situ. Science 343, 1360-1363 (2014).
15. Goltsev, Y. et al. Deep Profiling of Mouse Splenic Architecture with CODEX Multiplexed Imaging. Cell 174, 968-981.e15 (2018).
16. Keren, L. et al. A Structured Tumor-Immune Microenvironment in Triple Negative Breast Cancer Revealed by Multiplexed Ion Beam Imaging. Cell 174, 1373-1387.e19 (2018).
17. Merritt, C. R. et al. High multiplex, digital spatial profiling of proteins and RNA in fixed tissue using genomic detection methods. doi:10.1101/559021.
18. Codeluppi, S. et al. Spatial organization of the somatosensory cortex revealed by osmFISH. Nat. Methods 15, 932-935 (2018).
19. Kühnemund, M. et al. Targeted DNA sequencing and in situ mutation analysis using mobile phone microscopy. Nat. Commun. 8, 13913 (2017).
20. Ståhl, P. L. et al. Visualization and analysis of gene expression in tissue sections by spatial transcriptomics. Science 353, 78-82 (2016).
21. Rodrigues, S. G. et al. Slide-seq: A scalable technology for measuring genome-wide expression at high spatial resolution. Science 363, 1463-1467 (2019).
22. Vickovic, S. et al. High-density spatial transcriptomics arrays for in situ tissue profiling. doi:10.1101/563338.
23. Weinstein, J. A., Regev, A. & Zhang, F. DNA Microscopy: Optics-free Spatio-genetic Imaging by a Stand-Alone Chemical Reaction. Cell 178, 229-241.e16 (2019).
24. Turakhia, M. P. et al. Rationale and design of a large-scale, app-based study to identify cardiac arrhythmias using a smartwatch: The Apple Heart Study. Am. Heart J. 207, 66-75 (2019).
25. Lundin, S., Stranneheim, H., Pettersson, E., Klevebring, D. & Lundeberg, J. Increased throughput by parallelization of library preparation for massive sequencing. PLoS One 5, e10029 (2010).
26. Lennon, N. J. et al. A scalable, fully automated process for construction of sequence-ready barcoded libraries for 454. Genome Biol. 11, R15 (2010).
27. Jemt, A. et al. An automated approach to prepare tissue-derived spatially barcoded RNA-sequencing libraries. Sci. Rep. 6, 37137 (2016).
28. Berglund, E. et al. Spatial maps of prostate cancer transcriptomes reveal an unexplored landscape of heterogeneity. Nat. Commun. 9, 2419 (2018).
29. Asp, M. et al. Spatial detection of fetal marker genes expressed at low level in adult human heart tissue. Sci. Rep. 7, 12941 (2017).
30. Thrane, K., Eriksson, H., Maaskola, J., Hansson, J. & Lundeberg, J. Spatially Resolved Transcriptomics Enables Dissection of Genetic Heterogeneity in Stage III Cutaneous Malignant Melanoma. Cancer Res. 78, 5970-5979 (2018).
31. Maniatis, S. et al. Spatiotemporal dynamics of molecular pathology in amyotrophic lateral sclerosis. Science 364, 89-93 (2019).
32. Salmén, F. et al. Barcoded solid-phase RNA capture for Spatial Transcriptomics profiling in mammalian tissue sections. Nat. Protoc. 13, 2501-2534 (2018).
33. Fisher, S. et al. A scalable, fully automated process for construction of sequence-ready human exome targeted capture libraries. Genome Biol. 12, R1 (2011).
34. Rohland, N. & Reich, D. Cost-effective, high-throughput DNA sequencing libraries for multiplexed target capture. Genome Res. 22, 939-946 (2012).
35. Vickovic, S. et al. Massive and parallel expression profiling using microarrayed single-cell sequencing. Nat. Commun. 7, 13182 (2016).
36. Wong, K., Navarro, J. F., Bergenstråhle, L., Ståhl, P. L. & Lundeberg, J. ST Spot Detector: a web-based application for automatic spot and tissue detection for spatial Transcriptomics image datasets. Bioinformatics 34, 1966-1968 (2018).
37. Äijö, T. et al. Splotch: Robust estimation of aligned spatial temporal gene expression data. doi:10.1101/757096.
38. Lein, E. S. et al. Genome-wide atlas of gene expression in the adult mouse brain. Nature 445, 168-176 (2007).
39. Ellis, M. M., Ivan, J. S., Tucker, J. M. & Schwartz, M. K. rSPACE: Spatially based power analysis for conservation and ecology. Methods in Ecology and Evolution vol. 6 621-625 (2015).
40. Dobin, A. et al. STAR: ultrafast universal RNA-seq aligner. Bioinformatics 29, 15-21 (2013).
41. Anders, S., Pyl, P. T. & Huber, W. HTSeq—a Python framework to work with high-throughput sequencing data. Bioinformatics 31, 166-169 (2015).
42. Costea, P. I., Lundeberg, J. & Akan, P. TagGD: fast and accurate software for DNA Tag generation and demultiplexing. PLoS One 8, e57521 (2013).
43. Schindelin, J. et al. Fiji: an open-source platform for biological-image analysis. Nat. Methods 9, 676-682 (2012).
44. Svensson, V., Teichmann, S. A. & Stegle, O. SpatialDE: identification of spatially variable genes. Nat. Methods 15, 343-346 (2018).
45. Jones, E., Peterson, P. & Oliphant, T. SciPy: Open Source Scientific Tools for Python. Scipy http://www.scipy.org/(2001).

### Example 4—Spatial Proteomics and Combined Spatial Proteomics and Transcriptomics

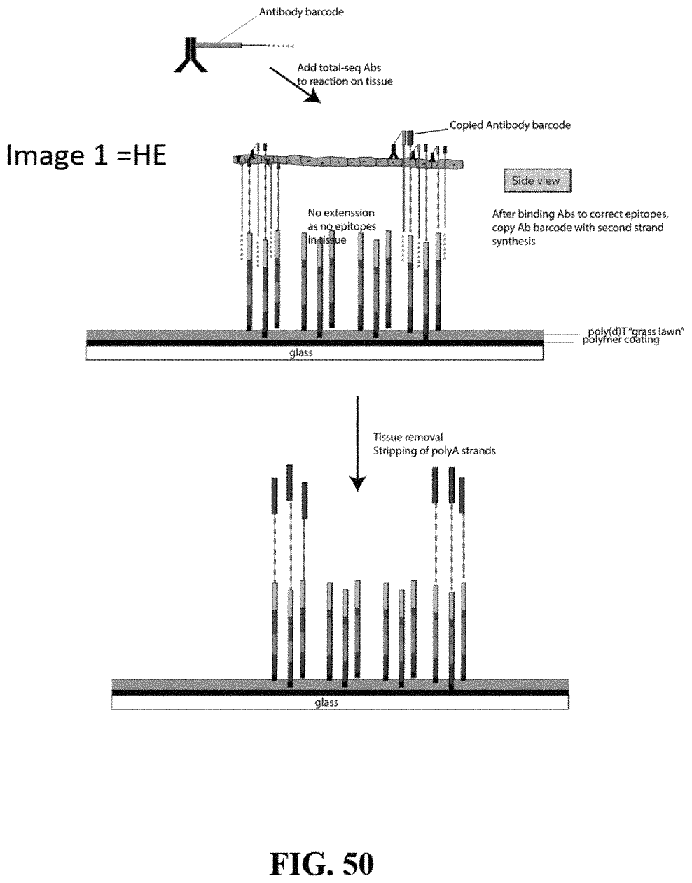

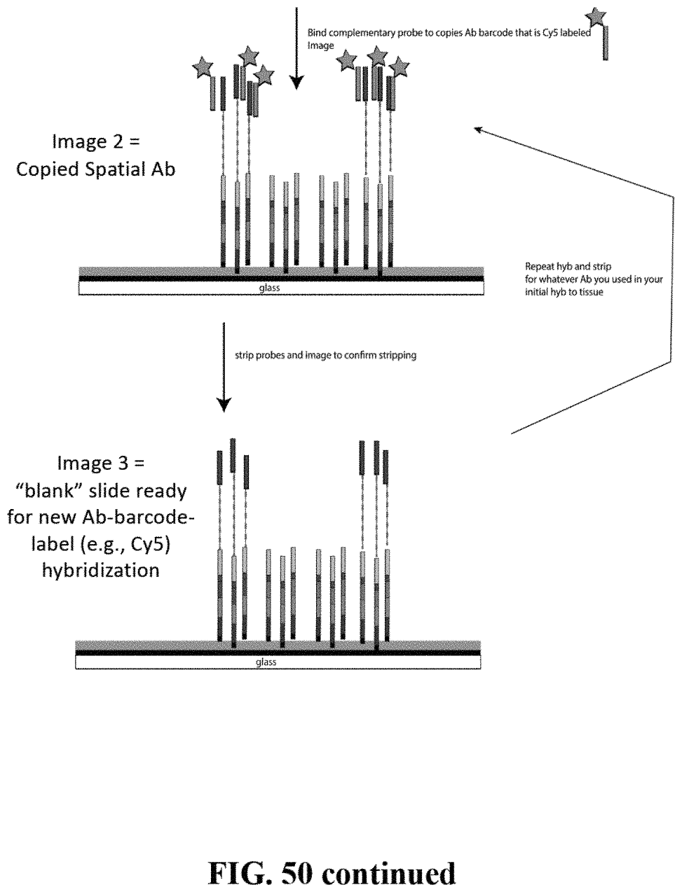

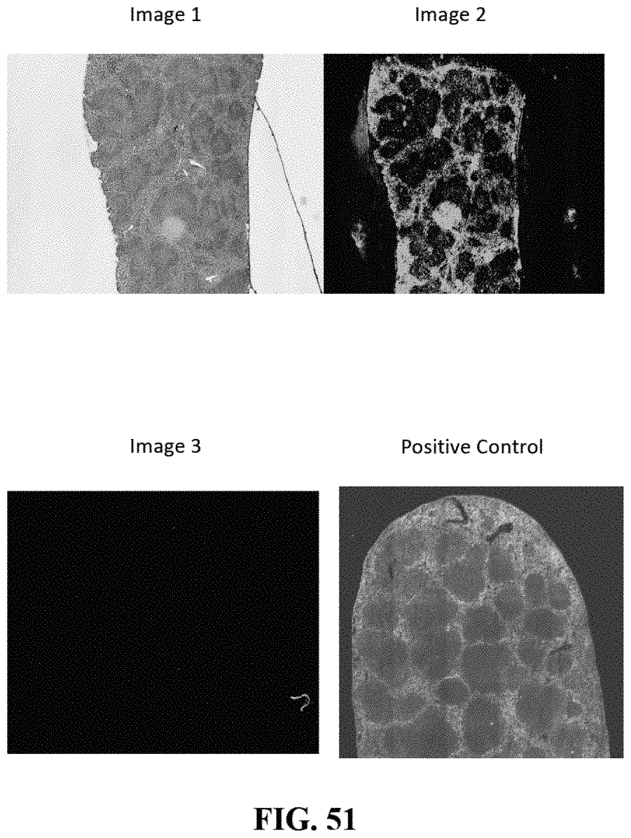

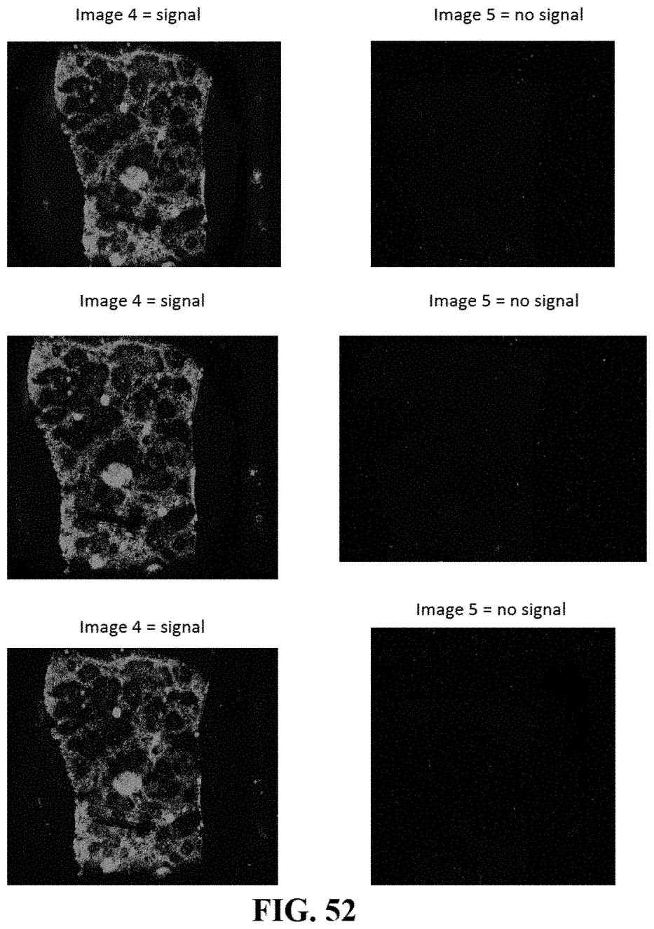

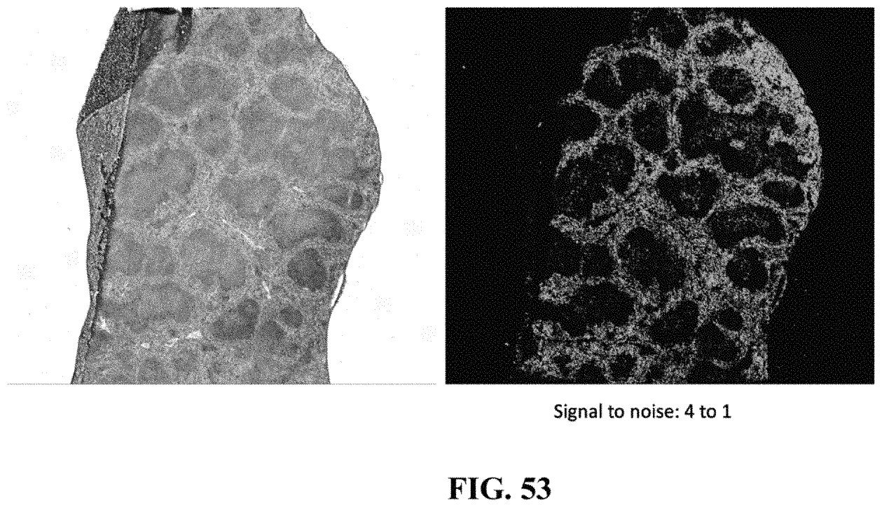

In general, FIGS. 50-64 show various embodiments of combining spatial proteomics and spatial transcriptomics to analyze, for example a complex tissue sample. FIG. 50 shows a general schematic of copying spatial antibody information into z=1 space and using imaging as a read-out. FIG. 51 shows Results on mouse spleen after performing the general scheme shown in FIG. 50 using Ab (F4/80 total-seq antibodies). Fluorescence (indicated by greyscale representative of green fluorescence) in images 2 and 3 indicate signal above threshold. The positive control shows regular antibody staining in the tissue using the same clone but with a Cy5-coupled antibody (Ab) as read out on an Epi scope. The pulp should stain red (represented in FIG. 51 in grayscale) based on the Cy5 label. FIG. 52 shows an iteration of images 2 and 3 (see e.g., FIG. 50) with the same fluorescent labeled-antibody-barcode. The surface appeared very stable, and results were reproducible between stripping cycles. Results shown are without any signal amplification. FIG. 53 demonstrates results from modifying reaction conditions were to improve signal to noise ratio to 4 to 1.

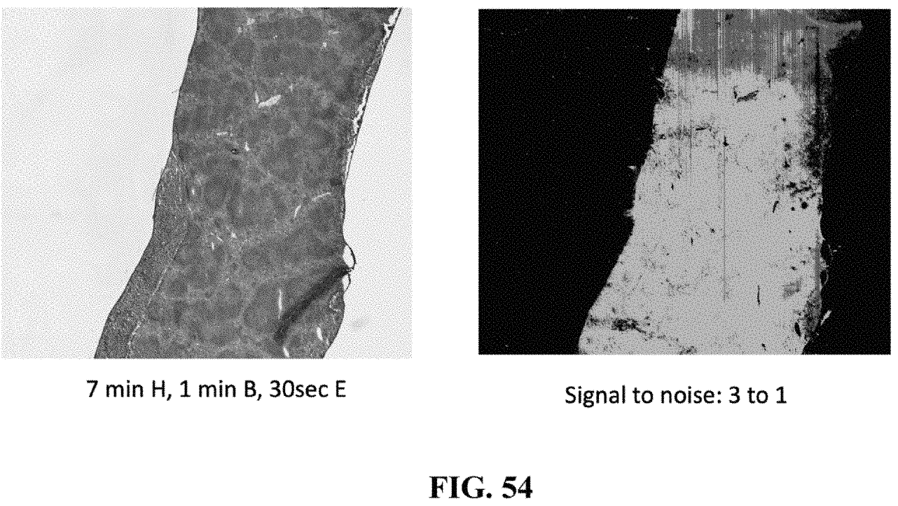

FIG. 54 shows a control that can demonstrate that antibody reaction conditions still generate an mRNA/cDNA print on the array surface. mRNA quality can be checked without adding any antibodies but with using the same reaction conditions.

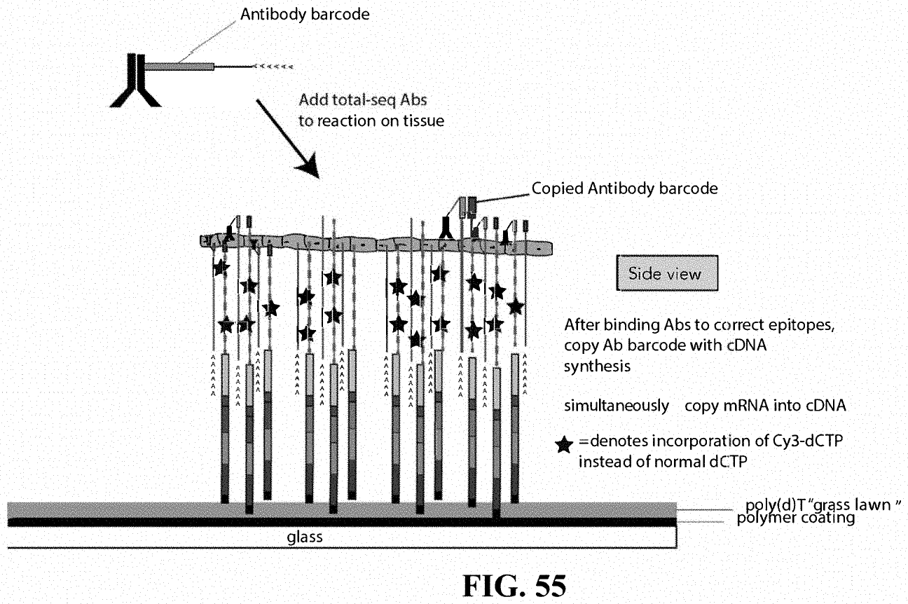

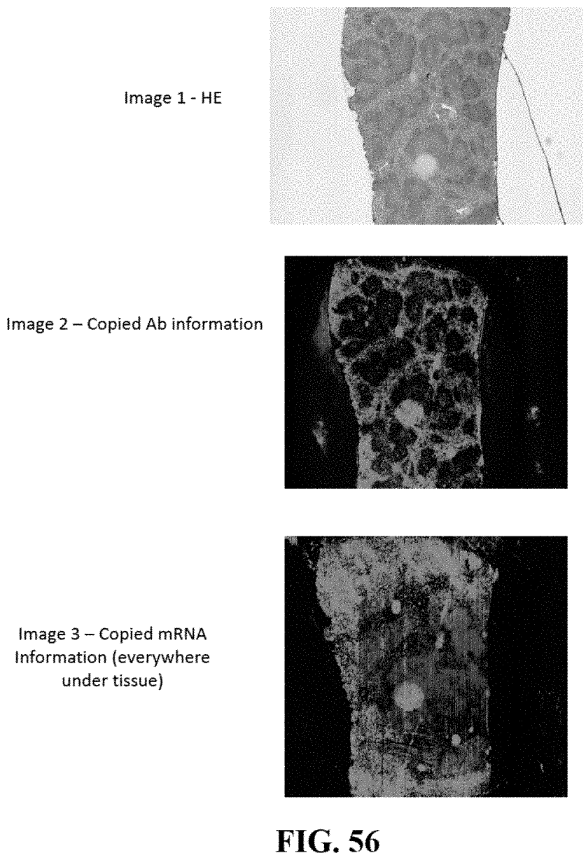

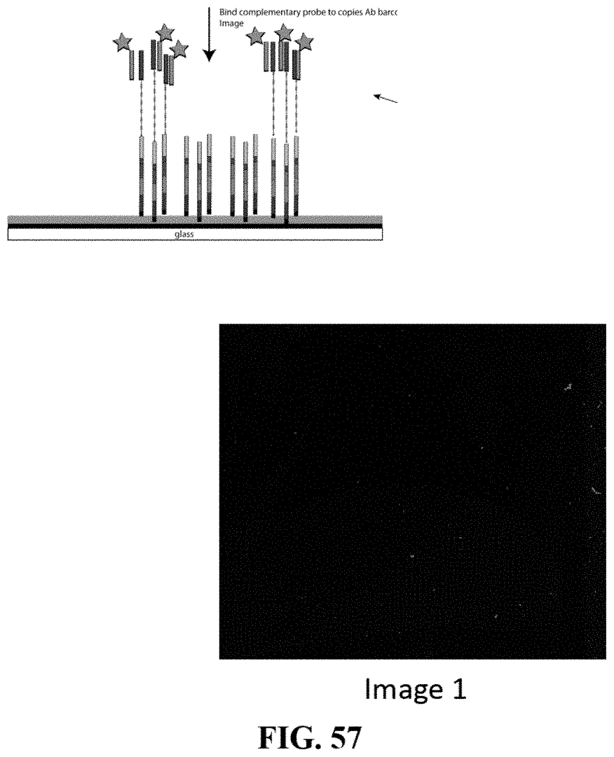

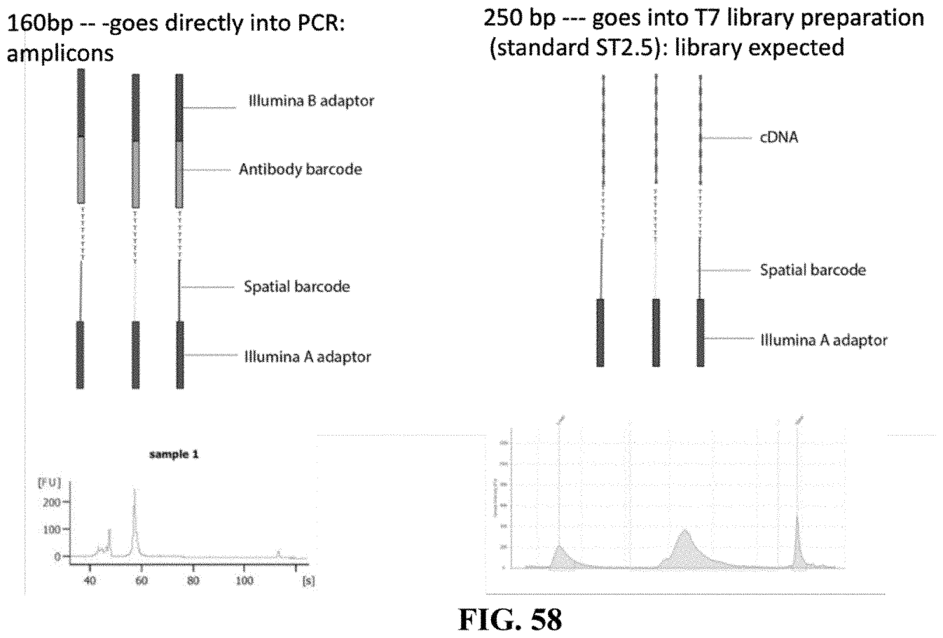

FIG. 55 shows a general scheme of combining spatial proteomics with transcriptomics on ST arrays. FIG. 56 shows results after imaging for copied antibody barcodes (same as general scheme shown in FIG. 50) and incorporated labeled dNTPs (Cy3 labeled dCTP). Fluorescence (indicated by greyscale representative of green fluorescence) in images 2 and 3 indicate signal above threshold. The same principle of detection was applied here as in FIGS. 50-54. In image 3, Cy3 signal (represented in greyscale) is for copied mRNA and Ab barcode as the barcode contains some cytosines (Cs). FIG. 57 shows that cDNA probes can be cleaved from the surface of the array for library creation. Imaging can check for how many probes are available after cDNA synthesis and cleaving of probes from the surface. A blank (dark) image indicates (e.g., image 1) that cDNA synthesis and cleavage of probes was successful. FIG. 58 shows validation of PCR products (antibody barcode information) and final ST2.5 cDNA libraries (mRNA information).

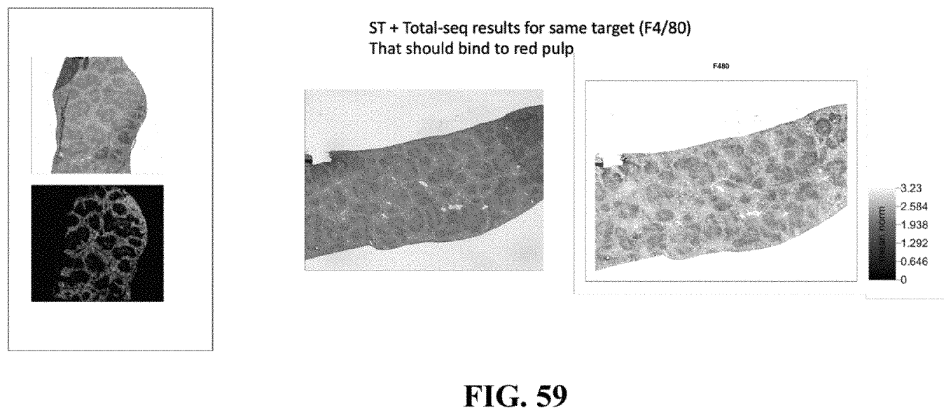

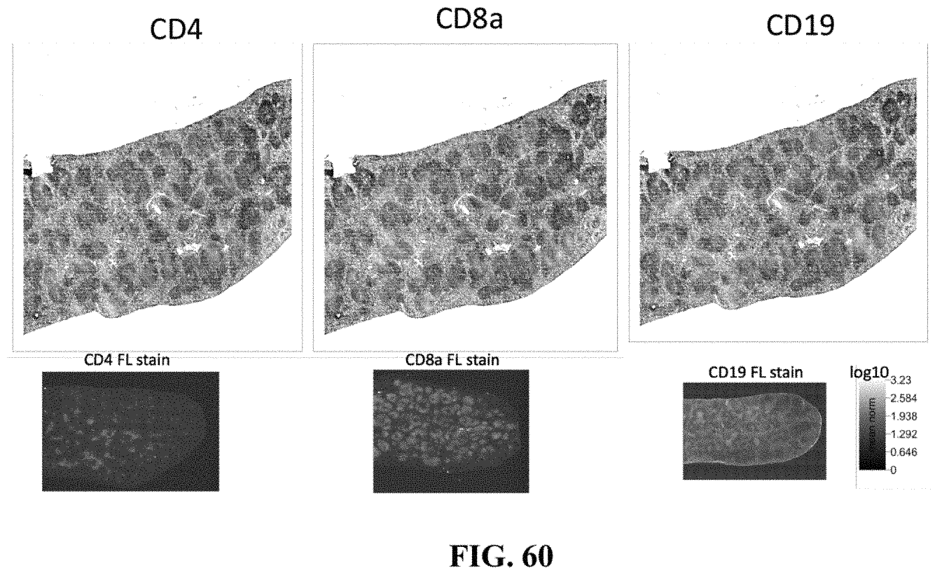

FIG. 59 shows combined spatial proteomics and transcriptomics POC sequencing results from combined spatial proteomics and transcriptomics on ST arrays (see general scheme in FIG. 50). FIG. 60 shows combined spatial proteomics and transcriptomics POC results for CD4, CD8a and CD19. Bottom images denote fluorescent staining images.

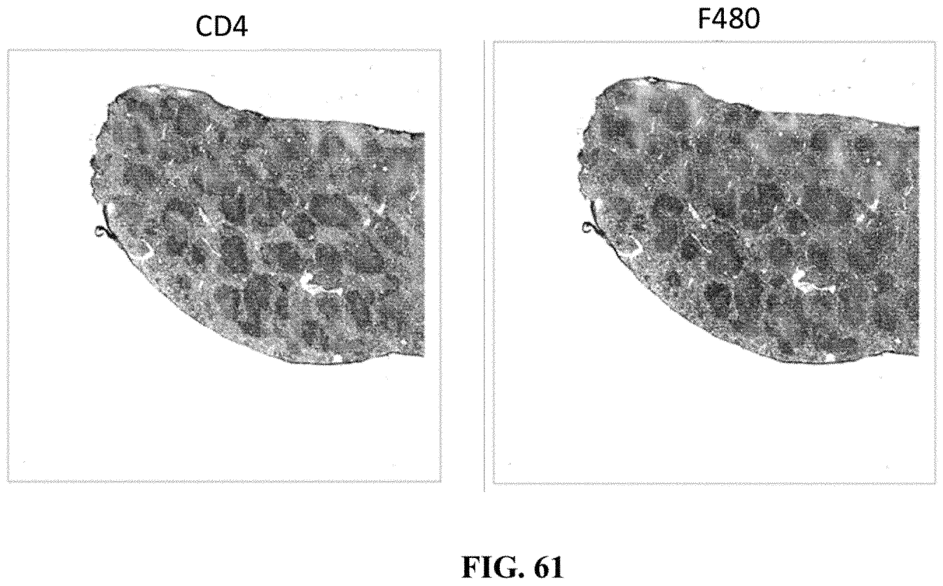

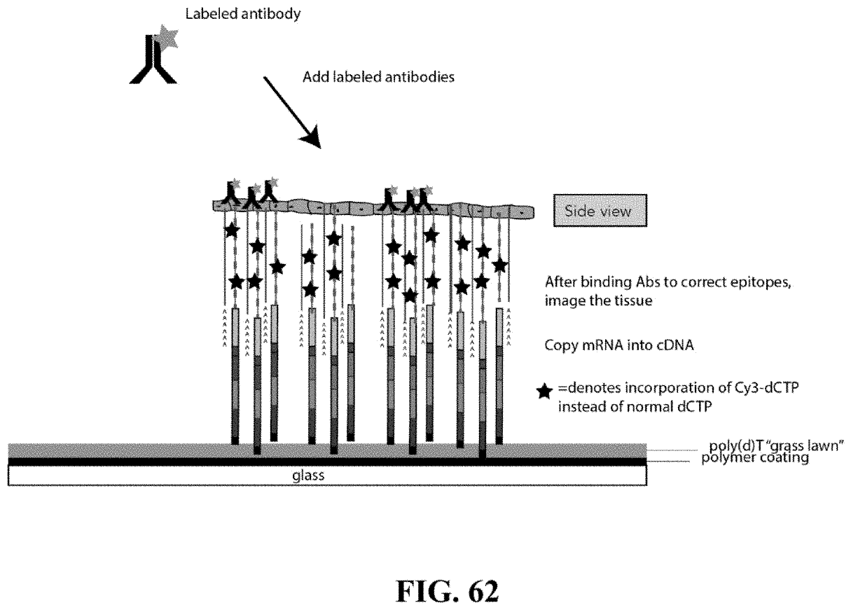

FIG. 61 shows combined spatial proteomics and transcriptomics POC results (replicate section based on 1M reads). FIG. 62 shows a general scheme for another embodiment of a combined spatial proteomics and transcriptomics using antibodies conjugated with fluorophores or other detectable label. This is combined with ST.

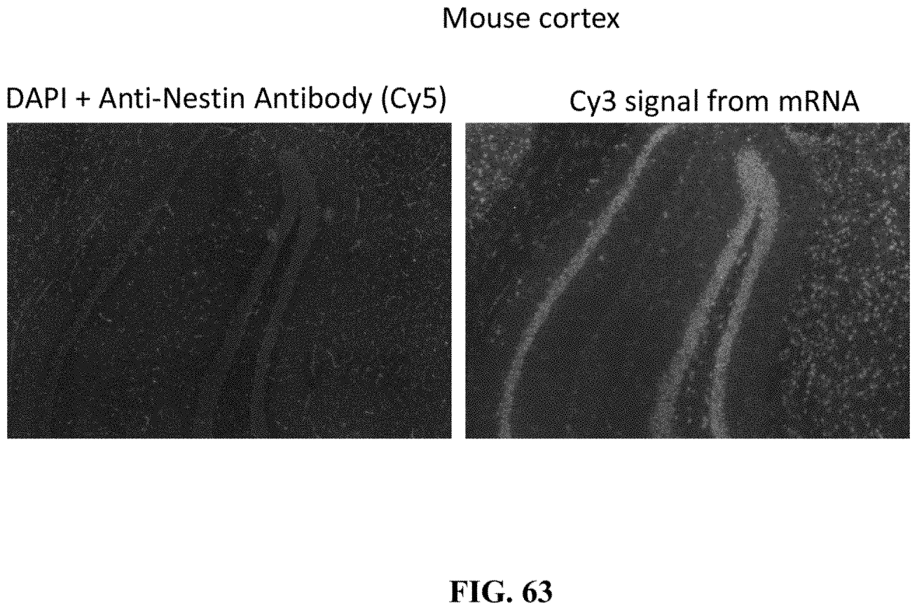

FIG. 63 shows combined spatial proteomics and transcriptomics POC results in mouse cortex after performing the general scheme set forth in FIG. 62. mRNA signal appeared very even throughout the tissue as indicated by the Cy3 signal. At this point, the Cy3 signal from mRNA should be observed everywhere throughout the tissue. Some loss of mRNA integrity was observed with each successive cycle.

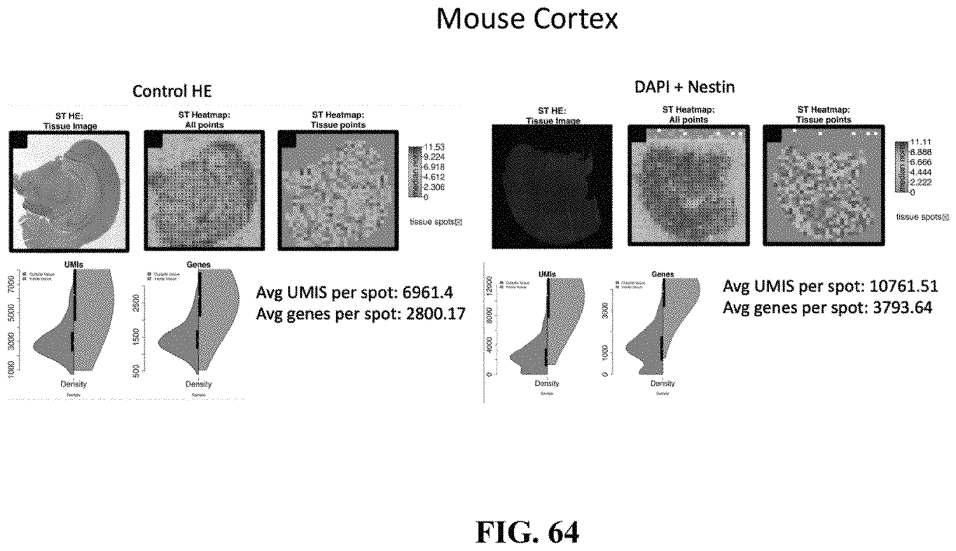

FIG. 64 shows combined spatial proteomics and transcriptomics on spatial transcriptomic arrays showing POC spatial sequencing results. These results in the mouse cortex (which applied the same sample in each, both prepped ST2.5) can confirm that the system works with fluorescent labels and produces even coverage under the tissue.

### Example 5—Magnetic Bead Array

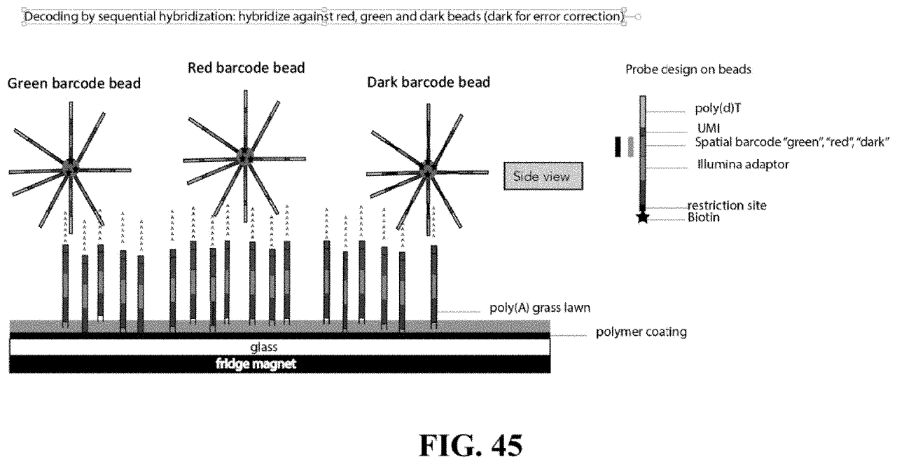

The array can be configured as a bead array. The bead array can include magnetic beads, which can inter alia allow for beads to be releasably coupled to or otherwise in contact with a sample or other component of the array. FIGS. 44-45 show a general scheme for a bead array. Magnetic beads having probes attached can be captured into an array using a magnet underneath a fixed array of probes on a substrate. The probes on the magnetic beads can have spatial barcodes that couple with different dyes (such as red and green as represented in greyscale) or none or a quencher (dark). Dark beads can be used for error and/or background correction. The bead array can be decoded by sequential hybridization against the different spatial barcodes on the beads.

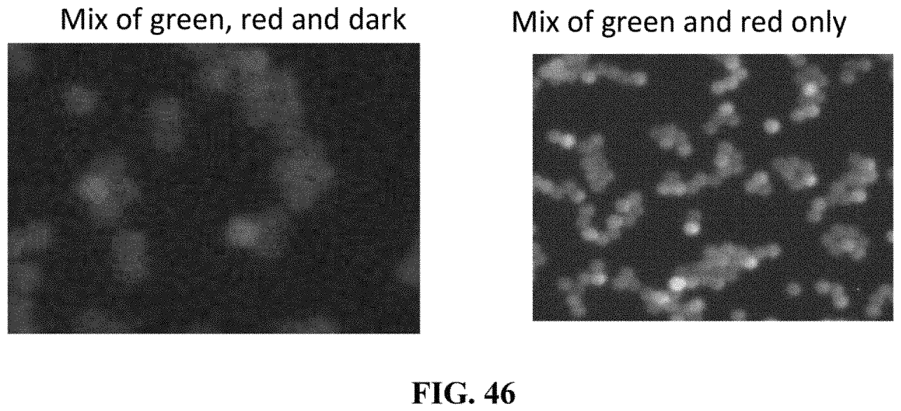

FIG. 46 shows an image of a bead array. Left image shows a mix of green, red, and dark beads. Right image shows a mix of green and red (as represented in greyscale) only. Dark is used to calculate errors and background.

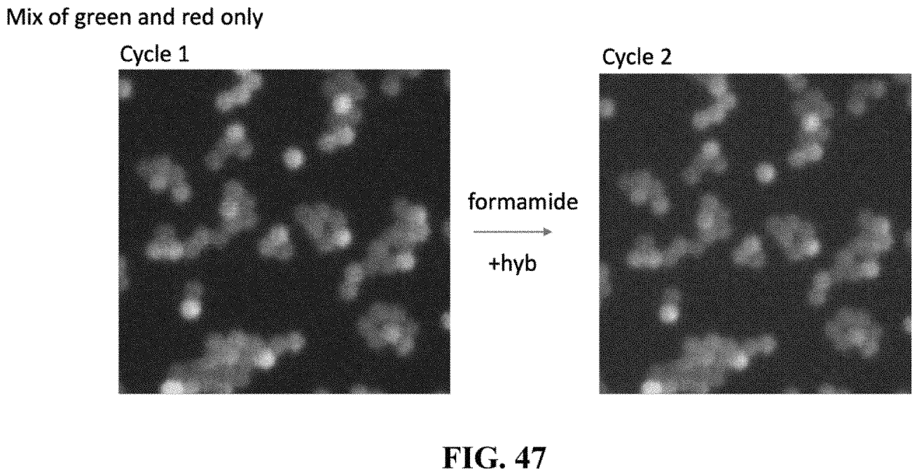

The bead arrays can be hybridized, stripped, and imaged repeatedly (FIG. 47). The cycle of hybridizing, stripping, and imaging for an entire slide filled with arrays is about 25 minutes. In some embodiments, multiple slides (e.g., 2 to about 48 with about 1-96 spatial arrays per slide) can be processed at once. As long as the magnet is present beneath the substrate, the beads do not substantially move during the process.

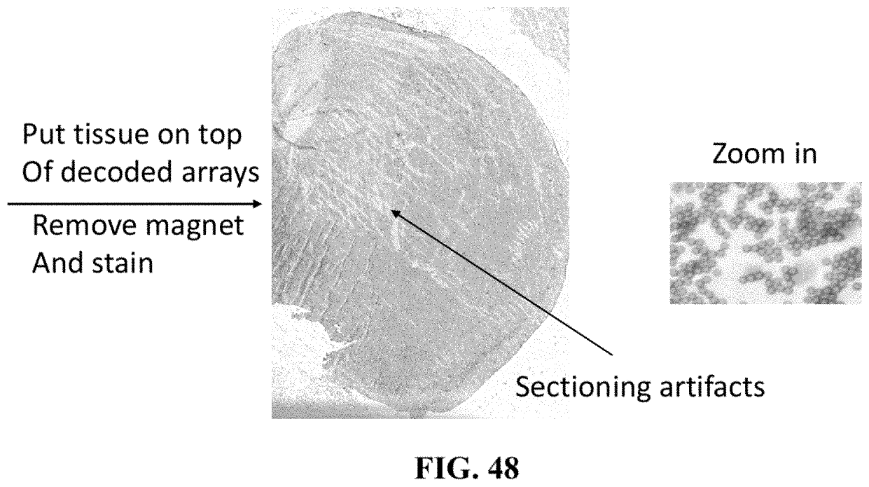

Tissue can be placed on top of the decoded arrays, the magnet can be removed, and the tissue can be stained (see e.g., FIG. 48) The beads can stick to the cells. The beads stick only to the closest cells/intact surfaces. The beads surrounding those stuck to the cells are washed away. Beads that are washed away can be captured and reused. The ability to capture and reuse the beads provides a cost and materials saving advantage not realized by other techniques. Sectioning artifacts can evidence that the beads stick only to the closest cells/intact surface.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of spatial and/or temporal processing of a sample comprising a plurality of cells comprising:
a. depositing a sample comprising a plurality of cells on a fixed addressable array or a decoded bead array,
i. wherein the fixed addressable array comprises:
a plurality of array probes, each array probe comprising a capture molecule and a spatial barcode, wherein each spatial barcode defines a unique x,y, position of each array probe in the fixed addressable array;
ii. wherein the decoded bead array comprises:
a plurality of conductive beads comprising a plurality of bead probes, each comprising a target molecule and a spatial barcode, wherein the plurality of conductive beads are transiently fixed in spatial position to a first side of the fixed addressable array by an electromagnetic field applied to a second side of the fixed addressable array and wherein the first side and the second side are opposite sides of the fixed addressable array, wherein the conductive beads are optionally magnetic beads; and
b. operatively coupling material from the sample to the plurality of array probes of the fixed addressable array or the plurality of bead probes of the decoded bead array, thereby linking the operatively coupled material from the sample with an x,y position in the fixed addressable array and/or the decoded bead array.

2. The method of claim 1, wherein operatively coupling material from the sample comprises:
directly capturing material, indirectly capturing material, or both from the sample by a capture molecule of an array probe that is in spatial proximity to the captured material, thereby linking the captured material from the sample with an x,y position in the fixed addressable array.

3. The method of claim 2, wherein directly capturing material from the sample comprises capturing a sample polynucleotide by hybridizing the sample polynucleotide to the capture molecule of the array probe that is in spatial proximity to the sample polynucleotide or binding a labeled recognition molecule to a target present in the sample, wherein the sample polynucleotide optionally is or comprises DNA, RNA, or both.

4.-5. (canceled)

6. The method of claim 2, wherein indirectly capturing material from the sample comprises
i. specifically binding a barcoded recognition molecule to a target present in the sample, wherein the barcoded recognition molecule comprises a recognition molecule barcode;
ii. optionally, specifically a binding non-barcoded recognition molecule comprising a detectable label to the target present in the sample; and
iii. capturing the barcoded recognition molecule barcode by the capture molecule of the array probe that is in spatial proximity to the target.

7. The method of claim 2, further comprising (a) copying the captured sample polynucleotide(s), the captured barcoded recognition molecule barcode(s), or both, thereby forming a copied sample polynucleotide(s), a copied barcoded recognition molecule barcode(s), or both, (b) detecting the copied sample polynucleotides, copied barcoded recognition molecule barcodes, or both, wherein detecting optionally comprises imaging the copied sample polynucleotides, imaging the copied barcoded recognition molecule barcodes, or both, or both (a) and (b).

8.-9. (canceled)

10. The method of claim 2, further comprising capturing an image of the sample on the fixed addressable array, and optionally annotating regions of the image of the sample, optionally based on morphology.

11. (canceled)

12. The method of claim 11, further comprising
correlating the directly captured material, indirectly captured material, or both to a position in the sample on the fixed addressable array, wherein correlating optionally comprises assigning pixel coordinates to the image of the sample, image of the copied sample polynucleotides, image of the copied barcoded recognition molecules, or a combination thereof and coordinating the assigned pixel coordinates to the x,y position in the fixed addressable array; and optionally assigning a cell type, cell state, or both to cells in the sample, staining the sample and optionally recording the morphology of the stained sample, permeabilizing the sample, or any combination thereof.

13.-16. (canceled)

17. The method of claim 7, wherein copying the captured sample polynucleotide(s), the captured barcoded recognition molecule barcode(s), or both, comprises incorporating labeled dNTP's into the copied sample polynucleotide(s), the copied barcoded recognition molecule barcode(s), or both,
wherein copying the captured sample polynucleotide(s) optionally comprises synthesizing a complementary strand from the array probe using the captured sample polynucleotide as a template, using the captured barcoded recognition molecule barcode as a template, or both.

18. The method of claim 17, wherein detecting the copied sample polynucleotides, copied barcoded recognition molecule barcodes, or both comprises detecting the labeled dNTPs incorporated into the copied sample polynucleotide(s), a copied barcoded recognition molecule barcode(s), or both, and wherein detecting the labeled dNTPs optionally comprises imaging the labeled dNTPs.

19.-21. (canceled)

22. The method of claim 7, further comprising specifically binding a concatemer to a copied barcode recognition molecule(s), copied sample polynucleotide(s), or both.

23. The method of claim 7, wherein the sample, the barcoded recognition molecule, captured barcoded recognition molecule barcode, captured sample polynucleotide or a combination thereof is/are removed prior to detecting the copied barcoded recognition molecule barcode, copied polynucleotide, or both.

24. The method of claim 7, wherein detecting the copied barcoded recognition molecule barcode, the copied sample polynucleotide, or both comprises specifically binding one or more—detectable probes to the copied barcoded recognition molecule barcode, the copied sample polynucleotide, concatemer, or a combination thereof, and optionally wherein
(a) detecting the copied barcoded recognition molecule barcode comprises specifically binding a first detectable probe to a first copied barcoded recognition molecule barcode corresponding to a first target and optionally specifically binding a second detectable probe to a second copied barcoded recognition molecule barcode corresponding to a second target,
(b) detecting the copied sample polynucleotide comprises specifically binding a first detectable probe to a first copied sample polynucleotide corresponding to a first sample polynucleotide and optionally specifically binding a second detectable probe to a second copied sample polynucleotide corresponding to a second sample polynucleotide,
(c) or both (a) and (b).

25.-26. (canceled)

27. The method of claim 24, wherein
(a) the first detectable probe specifically bound to the first copied barcoded recognition molecule barcode is removed prior to specifically binding the second detectable probe to the second copied barcoded recognition molecule barcode;
(b) the first detectable probe specifically bound to the first copied sample polynucleotide is removed prior to specifically binding the second detectable probe to the second copied sample polynucleotide; or
both (a) and (b).

28. The method of claim 27, wherein (a) specifically binding the first detectable probe to the first copied barcoded recognition molecule barcode and specifically binding the second detectable probe to the second copied barcoded recognition molecule barcode occurs simultaneously, (b) wherein specifically binding the first detectable probe to the first copied sample polynucleotide and specifically binding the second detectable probe to the second copied sample polynucleotide occurs simultaneously, or both (a) and (b).

29.-30. (canceled)

31. The method of claim 24, wherein the first detectable probe comprises a first label and the second detectable probe comprises a second label and wherein the first label and the second label are different or are the same.

32. (canceled)

33. The method of claim 6, wherein the detectable label on the optionally present non-barcoded recognition molecule is different than a first label or a second label present on the first or second detectable probes when present.

34. The method of claim 3, further comprising preparing a cDNA library from the captured sample polynucleotide or the copied barcoded recognition molecule barcode, wherein preparing the cDNA library comprises preparing a cDNA library PCR product, or both from the copied sample polynucleotide;
optionally releasing the copied sample polynucleotide and array probes or the copied barcoded recognition molecule barcodes and array probes from the fixed addressable array prior to generating a cDNAlibrary, PCR product, or both; and
optionally sequencing the cDNA library, PCR product, or both.

35.-39. (canceled)

40. The method of claim 34, further comprising correlating each of the cDNA molecules in the cDNA library, each PCR product, or both to a position in the sample on the fixed addressable array and optionally assigning a cell type, cell subtype, cell state, or any combination thereof to the plurality of cells in the sample, the assigning comprising detecting differential expression of the cDNA molecules, PCR product(s), or both, to generate a gene and/or protein signature and identifying cell type, cell subtype, cell state, or any combination thereof based on the gene signature at positions in the sample.

41. (canceled)

42. The method of claim 6, wherein the barcoded recognition molecule, the non-barcoded recognition molecule, or both comprise a polynucleotide guided nucleic acid targeting system or molecule thereof, an antibody or fragment thereof, an aptamer, or a combination thereof, wherein the polynucleotide guided nucleic acid targeting system or molecule thereof is optionally a CRISPR-Cas system.

43. (canceled)

44. The method of claim 1, further comprising sequencing the operatively coupled material.

45. (canceled)

46. The method of claim 1, wherein the fixed addressable array further comprises a substrate, wherein the plurality of array probes of the fixed addressable array are coupled to the substrate, and wherein the substrate comprises is optionally a solid substrate, a semi-solid substrate, a liquid substrate, or a hydrogel.

47. (canceled)

48. The method of claim 46, wherein the substrate comprises a polymer, wherein the polymer optionally forms a layer on a surface of the substrate, and wherein the plurality of array probes are coupled to the polymer.

49. The method of claim 46, wherein the substrate comprises a plurality of wells, wherein the plurality of wells is optionally organized in an array.

50. The method of claim 46, wherein the substrate comprises an optically transparent material.

51. The method of claim 46, further comprising releasing the plurality of array probes from the substrate, wherein releasing optionally comprises cleaving a cleavable linker on each array probe of the plurality of array probes.

52.-53. (canceled)

54. The method of claim 46, wherein further comprising depositing, optionally prior to depositing the sample on the fixed array, one or more CRISPR-Cas systems or components thereof onto the substrate, wherein the one or more CRISPR-Cas systems or components thereof are deposited at each x,y position defined by the fixed addressable array, and optionally wherein a guide sequence of the one or more CRISPR-Cas systems is coupled to an array probe in the plurality of array probes.

55.-59. (canceled)

60. The method of claim 1, wherein the sample is a tissue sample.

61. The method of claim 9, wherein detecting the copied sample polynucleotides, the labeled dNTPs, the copied barcoded recognition molecule barcodes, the sample, or any combination thereof comprises in-situ sequencing, laser scanning, fluorescent microscopy, DNA microscopy, FISH, smFISH, in situ PCR, or any combination thereof.

62. The method of claim 3, further comprising detecting the labeled recognition molecule, wherein detecting optionally comprises imaging the labeled recognition molecule.

63. The method of claim 3, wherein the labeled recognition molecule comprises a polynucleotide guided nucleic acid targeting system or molecule thereof, an antibody or fragment thereof, an aptamer, or a combination thereof, wherein the polynucleotide guided nucleic acid targeting system or molecule thereof is a CRISPR-Cas system.

64.-65. (canceled)

66. The method of claim 1, wherein operatively coupling the material from the sample to the bead array comprises depositing the sample comprising a plurality of cells on the decoded array and allowing at least some of the plurality of conductive beads to each couple to one or more of the plurality of cells.

67. (canceled)

68. The method of claim 67 wherein the spatial barcode is color coded or quenched and wherein decoding optionally comprises sequential hybridization and detection of the color coded or quenched spatial barcodes.

69. The method of claim 67, wherein the target molecule of at least one of the bead probes is captured by a capture probe of an array probe of the fixed addressable array, wherein decoding the bead array optionally comprises in-situ sequencing, laser scanning, DNA microscopy, fluorescent microscopy, laser scanning, FISH, smFISH, in-situ PCR, or a combination thereof.

70. (canceled)

71. The method of claim 68, further comprising calculating for background, calculating for errors, or both using the quenched spatial barcodes.

72.-73. (canceled)

74. The method of claim 1, wherein one or more array probes comprises an oligonucleotide sequence, wherein one or more bead probes comprises an oligonucleotide sequence, or both, and optionally wherein, one or more array probes, one or more bead probes, or both further comprise one or more of a unique molecular identifier (UMI), an adapter sequence, and a primer sequence.

75. The method of claim 1, wherein the capture molecule(s), the target molecule(s), or both comprises a Tn5 sequence, a 16S sequence, a poly(d)T sequence, a poly(d)A, sequence a random hexamer sequence, a trypsin molecule, an antibody, an aptamer, a Protein Epitope Signature Tag (PrEST) sequence, a DNA sequence or structural variation, or any combination thereof, wherein the DNA sequence or structural variation is optionally a single nucleotide polymorphism or a copy number variation.

76.-77. (canceled)

78. The method of claim 1, further comprising ablating a single layer of the sample and performing the step of operatively coupling material from the sample in a second layer of the sample.

79. (canceled)
